(19) United States
(12) Patent Application Publication
(10) Pub. No.: US 2025/0313647 A1
(43) Pub. Date: Oct. 9, 2025

(54) MUTANT AND MISLOCALIZED CELL SURFACE NUCLEOPHOSMIN 1 AS A DIAGNOSTIC AND THERAPEUTIC TARGET OF HUMAN DISEASE

(71) Applicants: The Children's Medical Center Corporation, Boston, MA (US); Cambridge Enterprise Limited, Cambridge (GB)

(72) Inventors: Ryan A. Flynn, Brookline, MA (US); Benson m. George, Boston, MA (US); Konstantinos Tzelepis, Cambridge (GB)

(73) Assignees: The Children's Medical Center Corporation, Boston, MA (US); Cambridge Enterprise Limited, Cambridge (GB)

(21) Appl. No.: 18/860,335

(22) PCT Filed: Apr. 27, 2023
(86) PCT No.: PCT/US2023/066299

§ 371 Date: Oct. 25, 2024

(30) Foreign Application Priority Data
Apr. 27, 2022 (GR) 20220100348

(51) Int. Cl. C07K 16/30; A61K 39/00; A61K 47/68; A61P 35/00; C12N 9/24; G01N 33/574

(52) U.S. Cl. CPC C07K 16/30; A61K 47/6815; A61K 47/6851; A61P 35/00; C12N 9/2497; C12Y 302/02022; G01N 33/57492; A61K 2039/505; C07K 2317/33; G01N 2333/705

(57) ABSTRACT

Provided herein are antibodies specific for nucleophosmin 1 (NPM1), which are capable of binding to wild-type and/or mutant nucleophosmin 1 located on the surface of cells. These antibodies, as well as antibody conjugates comprising these antibodies, are useful for the detection and treatment of cancers in which NPM1 is expressed on the surface of cancer cells.

### RELATED APPLICATION

This application claims the benefit of priority to Greece Application No. 20220100348, entitled “MUTANT AND MISLOCALIZED CELL SURFACE NUCLEOPHOSMIN 1 AS A DIAGNOSTIC AND THERAPEUTIC TARGET OF HUMAN DISEASE”, filed on Apr. 27, 2022, the contents of which are incorporated herein by reference in it's entirety.

### REFERENCE TO AN ELECTRONIC SEQUENCE LISTING

The contents of the electronic sequence listing (C123370224WO00-SEQ-VLJ.xml; Size: 47,034 bytes; and Date of Creation: Apr. 21, 2023) is herein incorporated by reference in its entirety.

### BACKGROUND OF INVENTION

Nucleophosmin 1 (NPM1) is a protein that typically resides in the nucleus and cytosol of cells. However, in certain cancers, such as acute myeloid leukemia (AML), NPM1 can be mislocalized to the cell surface. Some cancers are also characterized by a mutation in NPM1, which leads to the translation of a mutant NPM1, referred to as NPM1c, which is mislocalized to the cell surface in even greater quantities than wild-type NPM1. The NPM1c mutation is present in a considerable subpopulation of patients with AML.

### SUMMARY OF INVENTION

The present disclosure is based on the identification of antibodies that bind specifically to nucleophosmin 1 (NPM1). These antibodies are capable of targeting wild-type (WT) and/or mutant NPM1 located on the surface of cells, including cancer cells, and are useful for targeting cytotoxic payloads to cells with cell surface expression of WT and/or mutant NPM1.

Accordingly, some aspects of the present disclosure relate to an antibody that binds to NPM1.

In some embodiments, the antibody binds to WT NPM1. In some embodiments, the antibody comprises a heavy chain variable region comprising a heavy chain (HC) complementarity determining region (CDR) 1 comprising the amino acid sequence NIFVH (SEQ ID NO: 1), a HC CDR2 comprising the amino acid sequence KIDPANDNTKFAPNFQG (SEQ ID NO: 2), and a HC CDR3 comprising the amino acid sequence DSSGYDAVDY (SEQ ID NO: 3), and a light chain variable region comprising a light chain (LC) CDR1 comprising the amino acid sequence RASESVYTYLA (SEQ ID NO: 9), a LC CDR2 comprising the amino acid sequence NAKTLTE (SEQ ID NO: 10), and a LC CDR3 comprising the amino acid sequence QHHYGTPYT (SEQ ID NO: 11). In some embodiments, the heavy chain variable region comprises the amino acid sequence set forth in SEQ ID NO: 28 and/or the light chain variable region comprises the amino acid sequence set forth in SEQ ID NO: 29.

In some embodiments, the antibody binds to mutant NPM1. In some embodiments, the antibody comprises a heavy chain variable region comprising a heavy chain (HC) complementarity determining region (CDR) 1 comprising the amino acid sequence SYAMS (SEQ ID NO: 15), a HC CDR2 comprising the amino acid sequence AISGSGGSTYYADSVKG (SEQ ID NO: 16), and a HC CDR3 comprising the amino acid sequence WRNNAFDY (SEQ ID NO: 17), and a light chain variable region comprising a light chain (LC) CDR1 comprising the amino acid sequence QGDSLRSYYAS (SEQ ID NO: 22), a LC CDR2 comprising the amino acid sequence GKNNRPS (SEQ ID NO: 23), and a LC CDR3 comprising the amino acid sequence NSSPRLKHRVV (SEQ ID NO: 24). In some embodiments, the heavy chain variable region comprises the amino acid sequence set forth in SEQ ID NO: 30, and/or in the light chain variable region comprises the amino acid sequence set forth in SEQ ID NO: 31.

In some embodiments, the antibody is a full-length antibody or an antigen-binding fragment thereof.

In some embodiments, the antibody is a full-length antibody selected from an immunoglobulin G (IgG), an immunoglobulin A (IgA), an immunoglobulin D (IgD), an immunoglobulin E (IgE), and an immunoglobulin M (IgM). In some embodiments, the antibody is an IgG.

In some embodiments, the antibody is an antigen-binding fragment selected from a Fab fragment, a F(ab′)2 fragment, an Ig monomer, a Fd fragment, a scFv, a scAb, a dAb, a Fv, an affibody, a diabody, a single domain heavy chain antibody, and a single domain light chain antibody.

In some embodiments, the antibody is a human antibody or a humanized antibody.

In some embodiments, the antibody further comprises a heavy chain constant region. In some embodiments, the heavy chain constant region comprises the amino acid sequence set for in SEQ ID NO: 32 or SEQ ID NO: 46.

In some embodiments, the antibody further comprises a light chain constant region. In some embodiments, the light chain constant region comprises the amino acid sequence set forth in SEQ ID NO: 33, SEQ ID NO: 34 or SEQ ID NO: 47.

In some embodiments, the antibody comprises a heavy chain comprising the amino acid sequence of SEQ ID NO: 35 and/or a light chain comprising the amino acid sequence of SEQ ID NO: 37. In some embodiments, the antibody comprises a heavy chain comprising the amino acid sequence of SEQ ID NO: 36 and/or a light chain comprising the amino acid sequence of SEQ ID NO: 38. In some embodiments, the antibody comprises a heavy chain comprising the amino acid sequence of SEQ ID NO: 44 and/or a light chain comprising the amino acid sequence of SEQ ID NO: 45.

In some embodiments, the antibody preferentially binds to wild-type NPM1. In some embodiments, the antibody preferentially binds to mutant NPM1. In some embodiments, the antibody binds to both wild-type NPM1 and mutant NPM1.

Other aspects of the present disclosure relate to a composition comprising an antibody that binds NPM1 described herein. In some embodiments, the composition further comprises a pharmacologically acceptable excipient.

Other aspects of the present disclosure relate to a nucleic acid or nucleic acid set that encodes an antibody that binds NPM1 described herein. In some embodiments, the nucleic acid or nucleic acid set comprises a vector or vector set. In some embodiments, the vector or vector set is an expression vector or vector set.

Other aspects of the present disclosure relate to a cell comprising a nucleic acid or nucleic acid set that encodes an antibody that binds NPM1 described herein.

Other aspects of the present disclosure relate to a method of producing an antibody that binds NPM1 described herein, the method comprising culturing a cell that comprises a nucleic acid or nucleic acid set encoding an antibody that binds NPM1 described herein, collecting the cultured cells and/or culture media, and isolating the antibody from the cultured cells and/or culture media.

Other aspects of the present disclosure relate to a conjugate comprising an antibody that binds NPM1 described herein conjugated to an agent.

In some embodiments, the agent is a drug. In some embodiments, the drug is selected from the group consisting of: auristatin E, auristatin F, monomethyl auristatin D (MMAD), monomethyl auristatin F (MMAF), monomethyl auristatin E (MMAE), actinomycin, actinomycin X2, α-amanitin, β-amanitin, γ-amanitin, ε-amanitin, aeroplysinin, aldoxorubicin, agrochelin, ansatrienin, ansamitocin P-3, aphidicolin, apoptolidin, L-asparaginase, azacitidine, bafilomycin A1, bafilomycin B1, bafilomycin B2, bafilomycin C1, bafilomycin C2, bafilomycin D, bafilomycin E, calicheamicin, campathecin, chaetocin, chaetoglobosin, chlamydocin, cinerubin B, cladribine, colchicine, combretastatin A1, combretastatin A4, cordycepin, cryptophycin, cucurbitacin B, cucurbitacin E, curvulin, cyclopamine, cyclophosphamide, cytarabine, dactinomycin, daunorubicin, decitabine, dexamethasone, dolastatin 10, dolastatin 15, duocarmycin SA, duocarmycin TM, duocarmycin MA, duocarmycin DM, doxorubicin, englerin A, epothilone A, epothilone B, epothilone C, etoposide, fludarabine, fumagillin, geldanamycin, tanespimycin (17-AAG), glucopiericidin A, gramicidin A, herboxidiene, 9-hydroxyellipticine, hydroxyurea, hygrolidin, hypothemycin, idarubicin, ilimaquinone, isatropolone A, isofistularin-3, ixabepilone, JW55, lactacystin, luisol A, maytansinol, mertansine (DM1), maytansine DM3, ravtansine (DM4), maytansinoid AP-3, mechercharmycin A, mensacarcin, methotrexate, 6-mercaptopurine, microcolin B, microcystin LR, mitoxantrone, muscotoxin A, myoseverin, mytoxin B, nelarabine, nemorubicin, nocuolin A, okilactomycin, oligomycin A, oligomycin B, paclitaxel, larotaxel, milataxel, ortataxel, tesetaxel, phallacidin, phalloidin, phytosphingosine, piericidin A, pironetin, podophyllotoxin, polyketomycin, prednisone, pseudolaric acid B, pseurotin A, puwainaphycin F, pyrrolobenzodiazepine, quinaldopeptin, rachelmycin, rebeccamycin, Ro 5-3335, safracin B, sandramycin, sanguinarine, saporin, sinefungin, taltobulin, telomestatin, 6-thioguanine, thiocolchicine, tolytoxin, tripolin A, triptolide, tubastatin A, tubulysin A, tubulysin M, tubulysin IM-1, tubulysin IM-2, tubulysin IM-3, venetoclax, and vincristine. In some embodiments, the drug is saporin, daunorubicin, or venetoclax.

In some embodiments, the antibody and the drug are conjugated via a linker.

In some embodiments, the linker is a cleavable linker. In some embodiments, the linker is a pH-sensitive linker, a glutathione-sensitive linker, or a protease-cleavable linker. In some embodiments, the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, CL2A, maleimidocaproyl (MC), and Mal-EBE-Mal.

In some embodiments, the linker is a non-cleavable linker. In some embodiments, the non-cleavable linker is selected from the group consisting of: N-succinimidyl 4-(N-maleimidomethyl)cyclohexane-1-carboxylate (SMCC) and maleimidomethyl cyclohexane-1-carboxylate (MCC), MC-VC-PAB.

In some embodiments, the ratio of the antibody to the drug is between 1:1 and 1:10. In some embodiments, the ratio of the antibody to the agent is 1:4.

In some embodiments, the agent is a radioisotope. In some embodiments, the radioisotope is selected from the group consisting of: Iodine-131, Rhenium-188, Yttrium-90, Bismuth-213, and Actinium-225.

In some embodiments, the agent is an imaging agent. In some embodiments, the imaging agent is a luminescent or fluorescent imaging agent. In some embodiments, the imaging agent is an agent that is detectable by magnetic resonance imaging (MRI). In some embodiments, the imaging agent is gadolinium-diethylenetriamine (Gd-DTPA).

Other aspects of the present disclosure relate to a method of treating a nucleophosmin 1 (NPM1)-expressing cancer, the method comprising administering to a subject in need thereof an effective amount of an antibody or a conjugate described herein.

In some embodiments, the NPM1-expressing cancer is a cancer in which NPM1 is expressed on the surface of cancer cells. In some embodiments, the cancer is a solid or liquid cancer selected from the group consisting of: a hematological cancer, a lung cancer, a breast cancer, a brain cancer, a gastrointestinal cancer, a liver cancer, a kidney cancer, a bladder cancer, a pancreatic cancer, an ovarian cancer, a testicular cancer, a prostate cancer, an endometrial cancer, a muscle cancer, a bone cancer, a neuroendocrine cancer, a connective tissue cancer, a head or neck cancer, or a skin cancer. In some embodiments, the cancer is selected from the group consisting of: acute myeloid leukemia (AML), acute promyeloid leukemia (APL), acute lymphoblastic leukemia (ALL), non-Hodgkin's lymphoma, and myelodysplastic syndrome (MDS). In some embodiments, the cancer is a metastatic cancer. In some embodiments, the cancer is a therapy-related cancer or a secondary malignancy. In some embodiments, the cancer is therapy-related AML (t-AML) or a secondary malignancy of non-Hodgkin's lymphoma.

In some embodiments, the antibody or conjugate is administered systemically or locally. In some embodiments, the antibody or conjugate is administered via injection. In some embodiments, the injection is intravenous injection, subcutaneous injection, intraperitoneal injection, or intratumoral injection. In some embodiments, the antibody or conjugate is administered orally.

In some embodiments, the administration occurs more than once. In some embodiments, the administration occurs between once per day and once per six months.

In some embodiments, the subject is a mammal. In some embodiments, the subject is a human.

Other aspects of the present disclosure relate to a method for treating a disease associated with a cell expressing cell surface nucleophosmin 1 (NPM1), the method comprising administering to a subject in need thereof an effective amount of an antibody or a conjugate described herein that binds to NPM1.

Other aspects of the present disclosure relate to a method of evaluating the presence of a nucleophosmin 1 (NPM1)-expressing cancer, the method comprising administering to a subject in need thereof an effective amount of a conjugate described herein, imaging the conjugate in the subject, and determining the presence of a NPM1-expressing cancer in the subject, based on the level and position of the antibody conjugate imaged in the subject.

In some embodiments, the NPM1-expressing cancer is a cancer in which NPM1 is expressed on the surface of cancer cells. In some embodiments, the cancer is selected from the group consisting of: acute myeloid leukemia (AML), acute promyeloid leukemia (APL), acute lymphoblastic leukemia (ALL), non-Hodgkin's lymphoma, and myelodysplastic syndrome (MDS). In some embodiments, the cancer is a metastatic cancer. In some embodiments, the cancer is a therapy-related cancer or a secondary malignancy. In some embodiments, the cancer is therapy-related AML (t-AML) or a secondary malignancy of non-Hodgkin's lymphoma.

In some embodiments, the imaging is luminescent or fluorescent imaging. In some embodiments, the imaging is magnetic resonance imaging (MRI).

In some embodiments, the method further comprises administering to the subject an effective amount of an antibody or a conjugate described herein, if a NPM1-expressing cancer is determined to be present in the subject.

Other aspects of the present disclosure relate to a method of evaluating the presence of a nucleophosmin 1 (NPM1)-expressing cancer, the method comprising collecting a biological sample from a subject in need thereof, contacting the biological sample with a conjugate described herein, analyzing binding between the conjugate and NPM1-expressing cancer cells in the biological sample, and determining the presence of a NPM1-expressing cancer in the subject, based on the level of binding between the conjugate and NPM1-expressing cancer cells analyzed in the sample.

In some embodiments, the NPM1-expressing cancer is a cancer in which NPM1 is expressed on the surface of cancer cells. In some embodiments, the cancer is selected from the group consisting of: acute myeloid leukemia (AML), acute promyeloid leukemia (APL), acute lymphoblastic leukemia (ALL), non-Hodgkin's lymphoma, and myelodysplastic syndrome (MDS). In some embodiments, the cancer is a metastatic cancer. In some embodiments, the cancer is a therapy-related cancer or a secondary malignancy. In some embodiments, the cancer is therapy-related AML (t-AML) or a secondary malignancy of non-Hodgkin's lymphoma.

In some embodiments, the biological sample collected from the subject is a blood sample, a serum sample, or a plasma sample.

In some embodiments, the analysis is luminescent or fluorescent analysis. In some embodiments, the analysis is performed via flow cytometry.

In some embodiments, the method further comprises administering to the subject an effective amount of an antibody or a conjugate described herein, if a NPM1-expressing cancer is determined to be present in the subject.

### BRIEF DESCRIPTION OF DRAWINGS

The accompanying drawings are not intended to be drawn to scale. In the drawings, each identical or nearly identical component that is illustrated in various FIGS. is represented by a like numeral. For purposes of clarity, not every component may be labeled in every drawing.

In the drawings:

FIGS. 1A-1E show that NPM1 is localized on the surface of cancerous cell types. FIG. 1A shows immunoblotting of WT and mutant NPM1 in cytosolic and membrane fractions collected from various human leukemia cell lines. FIG. 1B shows FACS analysis of NPM1 surface expression in human leukemia cell lines. FIGS. 1C and 1D show relatively low levels of NPM1 expressed on the surface of murine bone marrow (BM) or peripheral blood (PB) cells. FIG. 1E shows FACS analysis that NPM1 is not robustly expressed on healthy human BM cells. FACS analysis was conducted with isolated anti-NPM1 antibodies, shown in FIG. 2A and FIG. 2B, as well as commercial anti-NPM1 antibody (Santa Cruz Biotechnology anti-WT NPM1 #sc-32256).

FIGS. 2A and 2B show preparation of anti-NPM1 antibodies. FIG. 2A shows isolation of an antibody that binds to WT NPM1. FIG. 2B shows isolation of an antibody that binds to mutant NPM1.

FIGS. 3A and 3B shows that an anti-NPM1-ADC is toxic toward human cancer cells. FIG. 3A shows cytotoxicity of an ADC comprising an antibody that binds WT NPM1 toward OCI-AML3 cells. Streptavidin-saporin was bound to either biotinylated anti-WT NPM1 or anti-mouse IgG, after which complexes were added to OCI-AML3 cells and incubated for 24 hours. After incubation, cells were washed and cell killing was analyzed by FACS. FIG. 3B shows the same as in FIG. 3A, with incubation for 48 hours.

FIGS. 4A and 4B show that NPM1 is highly conserved between mammalian species. FIG. 4A shows a pairwise alignment depicting conservation between human and murine NPM1 amino acid sequences (SEQ ID NOs: 39-40). FIG. 4B shows a pairwise alignment depicting conservation between human and Rhesus macaque NPM1 amino acid sequences (SEQ ID NOs: 39 and 41).

FIGS. 5A-5C shows that NPM1 is present on the surface of human AML cell lines and primary murine AML cells. FIG. 5A shows binding of an anti-NPM1 antibody (Merck/Sigma anti-B23 #B0556) to NPM1 localized on the surface of human OCI-AML3 cells and MOLM13 cells. Binding to NPM1 was assayed in both live cells and in fixed cells. OCI-AML3 cells express mutant NPM1, while MOLM13 cells express relatively lower amounts of WT NPM1. FIG. 5B and FIG. 5C show binding of an anti-NPM1 antibody (Merck/Sigma anti-B23 #B0556) to NPM1 localized on the surface of fixed primary murine MLL-AF4/FLT3^{ltd/+}, MLL-AF9/FLT3^{ltd/+}, MLL-ENL/FLT3^{ltd/+}, and Npm1c/FLT3^{ltd/+} AML cells.

FIGS. 6A-6F show that NPM1c (mutant NPM1) is present on the surface of a human myelogenous leukemia cell line. FIG. 6A shows that an anti-TY1 tag antibody (Diagenode TY1 #C15200054) binds the surface of human K562 myelogenous leukemia cells expressing TY1-tagged WT NPM1 or TY1-tagged NPM1c. FIG. 6B shows the data in FIG. 6A as separate panels with quantification of the percentage of cells positively bound by the TY1 antibody and anti-NPM1 antibody (Merck/Sigma anti-B23 #B0556). FIG. 6C shows binding of anti-NPM1 antibodies (Diagenode TY1 #C15200054; Merck/Sigma anti-B23 #B0556) to intracellular and cell surface WT and mutant NPM1. The relevant cells expressed an empty TY1 lentiviral vector, a TY1-tagged NPM1-wild-type lentiviral vector, or a TY1-tagged NPM1c lentiviral vector. FIG. 6D shows binding between anti-NPM1 antibodies (Diagenode TY1 #C15200054; Merck/Sigma anti-B23 #B0556) and intracellular WT and mutant NPM1. The relevant cells expressed an empty TY1 lentiviral vector, a TY1-tagged NPM1-wild-type lentiviral vector, or a TY1-tagged NPM1c lentiviral vector. FIG. 6E shows binding between anti-NPM1 antibodies (Diagenode TY1 #C15200054; Merck/Sigma anti-B23 #B0556) and WT and mutant NPM1 on the surface of K562 cells or MOLM13 cells (negative control). The relevant cells expressed an empty TY1 lentiviral vector, a TY1-tagged NPM1-wild-type lentiviral vector, or a TY1-tagged NPM1c lentiviral vector. FIG. 6F shows immunofluorescence depicting binding between anti-NPM1 antibodies (Diagenode TY1 #C15200054; Merck/Sigma anti-B23 #B0556) and WT and mutant NPM1 on the surface of K562 cells or MOLM13 cells (negative control). The relevant cells expressed an empty TY1 lentiviral vector, a TY1-tagged NPM1-wild-type lentiviral vector, or a TY1-tagged NPM1c lentiviral vector.

FIGS. 7A and 7B show that anti-NPM1 antibodies bind to NPM1 on the surface of primary murine AML cells. FIG. 7A shows binding between isolated WT and mutant anti-NPM1 antibodies and NPM1 localized on the surface of primary murine MLL-rearranged (MLL-r) AML cells. FIG. 7B shows binding between isolated WT and mutant anti-NPM1 antibodies and NPM1 localized on the surface of primary murine NPM1c AML cells.

FIGS. 8A-8D show that anti-NPM1 antibodies bind to NPM1 on the surface of human AML patient-derived xenografts (PDX) implanted in a murine model. FIG. 8A shows binding between isolated WT and mutant anti-NPM1 antibodies and NPM1 localized on the surface of PDX-1 cells (MLL-r). In contrast to PDX-1 cells, host (mouse) bone marrow cells (bottom panels) display relatively low binding. FIG. 8B shows binding between isolated WT and mutant anti-NPM1 antibodies and NPM1 localized on the surface of PDX-2 (NPM1c) cells. FIG. 8C shows binding between isolated WT and mutant anti-NPM1 antibodies and NPM1 localized on the surface of PDX-3 (MLL-r, BCOR) cells. FIG. 8D shows binding between isolated WT and mutant anti-NPM1 antibodies and NPM1 localized on the surface of PDX-4 (DNMT3A, N/KRAS) cells.

FIGS. 9A-9B show intracellular and cell surface staining of the isotype and NPM1 from cell line OCI-AML3 (FIG. 9A) and the denaturing protein gel and western blotting (WB) of biochemical fractionation (cytosol and membrane fractions) from four cell lines (FIG. 9B).

FIGS. 10A-10B show the results from live cell staining of cultured human suspension cell lines (FIG. 10A) and primary murine cell lines (FIG. 10B) with anti-NPM1 antibody (AF647 signal).

FIGS. 11A-11B show western blots of cells after cell surface biotinylation with a cell-impermeable biotinylation reagent. A western blot detects NPM1 in the membrane fraction of these cells and NPM1 IP is able to enrich NPM1 more robustly from the membrane lysate (FIG. 11A). Examination of the biotin signal from these fractions (cell surface exposed proteins) demonstrates the isolation of a single band in the NPM1 IP (FIG. 11B), showing that full length NPM1 is exposed to the surface of live cells.

FIGS. 12A-12B are diffraction limited (DL) and super resolution reconstructions (SR) of anti-NPM1 staining the cell surface of both HL-60 and OCI-AML3 (FIG. 12A) and the adherent cell line PANC1 (FIG. 12B). Cell surface NPM1 appears as distinct clusters.

FIG. 13 are fluorescent images of live (top) or fixed and permeabilized (bottom) OCI-AML3 cells stained with either Ab2.2 or commercially available NPM1 antibodies.

FIGS. 14A-14B shows three healthy donor bone marrow samples being stained with Ab2.2 and sorted for various markers of the hematopoietic system. Ab2.2 partially binds to CD33+ cells however there is no observable binding to CD34+(HSC) cells. FIG. 14B shows the binding of a commercial anti-NPM1 antibody as well as Ab2.2 binding to protein lysate samples by western blotting. The banding patterns are identical.

FIG. 15 is a table describing 12 AML patients.

FIGS. 16A-16B show the flow cytometry analysis of 12 AML.

FIG. 17 is a table describing samples collected from 15 AML patients.

FIGS. 18A-18E show the flow cytometry analysis of the 15 AML patients.

FIGS. 19A-19B show how the toxicity of Ab2.2 was assessed in vivo. FIG. 19A is a schematic of the experimental protocol. FIG. 19B are the weight results, and results from blood sample analysis of WBC, PLT and HGB.

FIGS. 20A-20G show how the efficacy of the Ab2.2 treatment was assessed in vivo. FIG. 20A is a schematic of the experimental design. FIG. 20B shows antibody binding to this AML model. FIG. 20C shows WBC count. FIG. 20D are results from qPCR analysis of the AML-allele. FIG. 20E shows spleens of IgG and Ab2.2 treated mice and the weight of the spleens. FIG. 20F are results of HGB and HCT analysis of blood samples. FIG. 20G show the results from a survival assay.

FIGS. 21A-21C shows how mice were subjected to sub-lethal irradiation and transplantation of primary murine AML cells to assess the efficacy (efficacy model 2). FIG. 21A is a schematic of the experimental design. FIG. 21B shows antibody binding. FIG. 21C show the results from a survival assay.

FIGS. 22A-22I show treatment with Ab2.2 reduces tumor burden in transplantation model. FIG. 22A is a schematic of the experimental design to test the effect of secondary transplant after Ab2.2 treatment. FIG. 22B are weights of the spleen, lung and liver in mice who received a transplant from IgG or Ab2.2 treated mice. FIG. 22C show the results from a survival assay. FIGS. 22D-22E shows flow cytometry results. FIG. 22F are the WBC count and PLT levels from blood samples. FIG. 22G shows the WBC over time. FIG. 22H shows images of the spleen and graphs of weight of spleens in isotype or Ab2.2 treated mice. FIG. 22I shows the AML % in BM and PB.

FIGS. 23A-23B shows immune dependency for tumor killing activity of Ab2.2; it on an intact immune system. FIG. 23A shows the experimental design. FIG. 23B shows the survival curve of either Ab2.2 or IgG treated mice.

FIGS. 24A-24C shows the solid tumor activity of Ab2.2. FIG. 24A shows the experimental design. FIG. 24B shows the tumor volume on day 10 and day 13. FIG. 24C shows the tumor volume over time from day 7 to day 13.

FIGS. 25A-25B shows wild type, healthy mice that were treated with sub-lethal irradiation and Ab2.2 did not exhibit an observable effect of Ab2.2 treatment. FIG. 25A shows the experimental design. FIG. 25B shows the weight and WBC count, HGB and PLT analysis from blood samples.

FIGS. 26A-26O show in vitro models of various cancer cell types that were analyzed using flow cytometry to assess the ability of Ab2.2 to bind to human or murine tumors.

FIG. 27 shows high surface detection of Npm1c on single Dmnt3a R882H mutant cells (pre-leukemia cells).

FIG. 28 shows how NPM1 can serve as an ADC target in some models. OCI-AML3 cells were treated with negative control, isotype Saporin conjugate or Ab2.2-Saporin conjugate to assess the in vitro activity of Ab2.2 with an ADC.

FIGS. 29A-29C show Ab2.2 extends lifespan in a transplantation model of human AML cell line. FIG. 29A is a schematic of the experimental design. FIG. 29B shows antibody binding using flow cytometry. FIG. 29C is a graph of the results of a survival assay.

FIGS. 30A-30C show Ab2.2 extends lifespan in a transplantation model of human AML-PDX cell line. FIG. 30A is a schematic of the experimental design. FIG. 30B shows antibody binding using flow cytometry. FIG. 30C is a graph of the results of a survival assay.

### DETAILED DESCRIPTION OF CERTAIN EMBODIMENTS

Aspects of the present disclosure are based on the discovery antibodies specific for nucleophosmin 1 (NPM1), also known as nucleolar phosphoprotein B23 or numatrin, may be used to target agents (e.g., chemotherapeutic drugs, radioactive isotopes) to certain cell types (e.g., cancer cells) in a subject. Without wishing to be bound by theory, NPM1 is a multifunctional protein that typically binds to single-stranded and double-stranded nucleic acids and is present in the nucleus and cytosol of cells. However, when certain cell types are exposed to cellular stress, such as cancerous transformation, quantities of NPM1 may instead be mislocalized to the cell surface where they are exposed to the extracellular environment. Although an increase in cell membrane localized NPM1 can occur in any type of cancer, it is particularly common in leukemia (e.g., acute myeloid leukemia (AML)) due to a mutation that occurs in the gene encoding NPM1. As a result of this mutation, leukemia (e.g., AML) cells in many patients begin expressing a mutant variant of NPM1, referred to as NPM1c, which is mislocalized from the nucleus to the cytoplasm, where it is trafficked to the cell membrane and exposed to the extracellular environment. NPM1c is also only known to occur in cancer cells, as it drives malignant transformation in AML. In principle, an antibody or other agent that is specific for wild-type NPM1 or mutant NPM1 (NPM1c) could be conjugated to a cytotoxic payload and used to target cancer cells. Alternately, an NPM1-specific antibody may itself be sufficient to stimulate antibody dependent cellular cytotoxicity (ADCC) or antibody dependent cell phagocytosis (ADCP) upon binding to cancer cells, without conjugation to a cytotoxic payload. However, while antibodies specific for NPM1 are commercially available, antibodies that can effectively bind to wild-type and/or mutant NPM1 on the surface of live cells are lacking. Additionally, most commercially available anti-NPM1 antibodies are polyclonal and there are currently no available monoclonal antibodies that bind to NPM1c. Therefore, new antibodies have been developed that are capable of effectively and specifically binding to wild-type NPM1 and mutant NPM1 on the surface of cells, including cancer cells. These antibodies and molecular conjugates thereof (e.g., antibody-drug conjugates) may be useful for detecting and treating cancers expressing NPM1 on their cell surfaces, and may be used to treat subjects with cancer for which there is no known effective treatment.

### Antibodies Binding NPM1

The present disclosure provides antibodies that bind to NPM1, for example, wild-type (WT) NPM1 or mutant NPM1. Such antibodies may have higher affinity for WT NPM1 than for mutant NPM1, or may have higher affinity for mutant NPM1 than for WT NPM1.

As used herein, the term “antibody” refers to an immunoglobulin molecule capable of specific binding to a target, referred to as an “antigen,” such as but not limited to a protein or peptide, through at least one recognition site on the antigen. As used herein, the term “antibody” encompasses both full-length immunoglobulin molecules (immunoglobulins having two heavy chains and two light chains, e.g., a monoclonal or polyclonal full-length antibody) and antigen-binding fragments thereof, such as, but not limited to chimeric antibodies, diabodies, linear antibodies, nanobodies, single chain antibodies, multispecific antibodies (e.g., bispecific antibodies), or another antigen-binding fragment that retains specific binding for the antigen. An “immunoglobulin (Ig)” is a large, Y-shaped protein produced mainly by plasma cells that is used by the immune system to neutralize an exogenous substance (e.g., a pathogen such as bacteria and viruses). “Antibody fragments” include any antigen binding fragment (i.e., “antigen-binding portion”) or single chain thereof. In some embodiments, an “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H} 1, C_{H} 2 and C_{H} 3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In some embodiments, an antibody is an immunoglobulin (Ig) monomer. An antibody may be a polyclonal antibody or a monoclonal antibody.

In some embodiments, an antibody is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain. Particular amino acid residues are believed to form an interface between the light chain and heavy chain variable domains. The pairing of a V_{H} and V_{L} together forms a single antigen-binding site. For the structure and properties of non-limiting examples of different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6, incorporated herein by reference. In some embodiments, an antibody is an IgG.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a 3-sheet configuration, connected by three hypervariable regions, which form loops connecting, and in some cases forming part of, the 3-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC) and/or antibody dependent cell phagocytosis (ADCP).

In some embodiments, the antibody is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). Monoclonal antibodies may also be isolated from phage antibody libraries, e.g., using the techniques described in Clackson et al., Nature, 352:624-628 (1991) and Marks et al., J. Mol. Biol., 222:581-597 (1991), incorporated herein by reference.

The monoclonal antibodies described herein encompass “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g., Old World Monkey, Ape etc.), and human constant region sequences.

In some embodiments, the antibody is a polyclonal antibody. A “polyclonal antibody” is a mixture of different antibody molecules which react with more than one immunogenic determinant of an antigen. Polyclonal antibodies may be isolated or purified from mammalian blood, secretions, or other fluids, or from eggs. Polyclonal antibodies may also be recombinant. A recombinant polyclonal antibody is a polyclonal antibody generated by the use of recombinant technologies. Recombinantly generated polyclonal antibodies usually contain a high concentration of different antibody molecules, all or a majority of (e.g., more than 80%, more than 85%, more than 90%, more than 95%, more than 99%, or more) which are displaying a desired binding activity towards an antigen composed of more than one epitope.

In some embodiments, the antibodies are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Some aspects of the present disclosure relate to an antibody that binds to NPM1. Such an antibody may preferentially bind to WT NPM1 or mutant NPM1. The term “preferentially binds” refers to binding that occurs more frequently, at a higher rate, over a greater duration, and/or with greater affinity with a particular antigen than with other antigens. The terms “preferentially binds” and “specifically binds” may be used interchangeably. The terms “preferentially binds” and “specifically binds” do not necessarily denote exclusive binding, i.e., an antibody that preferentially bind to an antigen may or may not bind to one or more additional antigens. In some embodiments, an antibody described herein may or may not bind to other forms (variants) of a specific antigen. In some embodiments, an antibody that binds to WT NPM1 may or may not bind to a variant of NPM1 comprising one or more amino acid insertions, deletions, or substitutions (i.e., a mutant NPM1), and a variant of NPM1 comprising one or more amino acid insertions, deletions, or substitutions (i.e., a mutant NPM1) may or may not bind to WT NPM1.

As used herein, “wild-type NPM1” or “WT NPM1” refers to a NPM1 protein comprising an amino acid sequence that is identical to that of a generally accepted reference sequence for intact, fully functional NPM1. For example, a WT NPM1 may be an NPM1 isoform (e.g., NCBI Reference Sequence: NP_001341935.1) that is expressed from a gene encoding WT NPM1 (NCBI Reference Sequence: NG_016018.1). As used herein, a “mutant NPM1” refers to a NPM1 protein comprising an amino acid sequence that comprises one or more amino acid insertions, deletions, or substitutions, relative to a WT NPM1 sequence. For example, a mutant NPM1 may comprise 1, 2, 3, 4, 5, 6, 7, 8, 9, 10, or more amino acid insertions, deletions, or substitutions, relative to a WT NPM1 sequence. A mutant NPM1 may have reduced activity and/or altered subcellular localization compared to WT NPM1. A WT NPM1 or mutant NPM1 described herein may be a mammalian WT NPM1 or mammalian mutant NPM1. In some embodiments, a WT NPM1 or mutant NPM1 described herein is a human WT NPM1 or human mutant NPM1.

In some embodiments, an antibody that binds to NPM1 is a full-length antibody. A full-length antibody described herein may be of any antibody class based on the amino acid sequence of its heavy chain constant region. For example, a full-length antibody that binds to NPM1 may be an immunoglobulin G (IgG), an immunoglobulin A (IgA), an immunoglobulin D (IgD), an immunoglobulin E (IgE), and an immunoglobulin M (IgM), or a subclass thereof (e.g., IgG1, IgG2, IgG3, IgG4). In some embodiments, an antibody described herein is an antigen-binding fragment. An antigen-binding fragment may be in the format of a Fab fragment, a F(ab′)2 fragment, an Ig monomer, a Fd fragment, a scFv, a scAb, a dAb, a Fv, an affibody, a diabody, a single domain heavy chain antibody, and a single domain light chain antibody. Antibodies described herein may be of murine, rat, human, or any other origin. In some embodiments, an antibody described herein is a human antibody or a humanized antibody.

In some embodiments, an antibody that binds to NPM1 (e.g., WT NPM1 or mutant NPM1) comprises a heavy chain variable region and a light chain variable region. In some embodiments, the heavy chain variable region and light chain variable region each comprise a set of complementarity determining region (CDR) sequences that determine substrate specificity. CDR sequences may be determined using any numbering scheme that is generally known in the art (e.g., Kabat, Chothia, Contact, IGMT).

In some embodiments, an antibody that binds to NPM1 comprises a heavy chain variable region comprising a heavy chain CDR1 comprising the amino acid sequence NIFVH (SEQ ID NO: 1), a HC CDR2 comprising the amino acid sequence KIDPANDNTKFAPNFQG (SEQ ID NO: 2), and a HC CDR3 comprising the amino acid sequence DSSGYDAVDY (SEQ ID NO: 3). In some embodiments, an antibody that binds to NPM1 comprises a light chain comprising a light chain CDR1 comprising the amino acid sequence RASESVYTYLA (SEQ ID NO: 9), a LC CDR2 comprising the amino acid sequence NAKTLTE (SEQ ID NO: 10), and a LC CDR3 comprising the amino acid sequence QHHYGTPYT (SEQ ID NO: 11).

In some embodiments, an antibody that binds to NPM1 comprises a heavy chain variable region comprising a heavy chain CDR1 comprising the amino acid sequence SYAMS (SEQ ID NO: 15), a HC CDR2 comprising the amino acid sequence AISGSGGSTYYADSVKG (SEQ ID NO: 16), and a HC CDR3 comprising the amino acid sequence WRNNAFDY (SEQ ID NO: 17). In some embodiments, an antibody that binds to NPM1 comprises a light chain variable region comprising a light chain CDR1 comprising the amino acid sequence QGDSLRSYYAS (SEQ ID NO: 22), a LC CDR2 comprising the amino acid sequence GKNNRPS (SEQ ID NO: 23), and a LC CDR3 comprising the amino acid sequence NSSPRLKHRVV (SEQ ID NO: 24).

Table 1 provides the amino acid sequences for heavy chain and light chain CDRs for exemplary antibodies (“Ab1” and “Ab2”) that are specific for NPM1 (WT and/or mutant NPM1). Antibodies that have the same heavy chain or light chain CDR sequences as provided in Table 1, as determined using any numbering scheme that is generally known in the art (e.g., Kabat, Chothia, Contact, IGMT), are within the scope of the present disclosure.

TABLE 1
CDR sequences of anti-NPM1 antibodies

| CDRs | Kabat | Chothia | Contact |
|---|---|---|---|
| CDR-H1 | NIFVH (SEQ ID NO: 1) | GFNIKNI (SEQ ID NO: 4) | KNIFVH (SEQ ID NO: 6) |
| (Ab1) | | | |
| CDR-H2 | KIDPANDNTKFAPNFQG | DPANDN (SEQ ID NO: | WIGKIDPANDNTK |
| (Ab1) | (SEQ ID NO: 2) | 5) | (SEQ ID NO: 7) |
| CDR-H3 | DSSGYDAVDY (SEQ ID | DSSGYDAVDY (SEQ ID | ASDSSGYDAVD (SEQ |
| (Ab1) | NO: 3) | NO: 3) | ID NO: 8) |
| CDR-L1 | RASESVYTYLA (SEQ ID | RASESVYTYLA (SEQ | YTYLAWY (SEQ ID NO: |
| (Ab1) | NO: 9) | ID NO: 9) | 12) |
| CDR-L2 | NAKTLTE (SEQ ID NO: | NAKTLTE (SEQ ID NO: | LLVYNAKTLT (SEQ ID |
| (Ab1) | 10) | 10) | NO: 13) |
| CDR-L3 | QHHYGTPYT (SEQ ID | QHHYGTPYT (SEQ ID | QHHYGTPY (SEQ ID |
| (Ab1) | NO: 11) | NO: 11) | NO: 14) |
| CDR-H1 | SYAMS (SEQ ID NO: 15) | GFTFSSY (SEQ ID NO: | SSYAMS (SEQ ID NO: |
| (Ab2 and Ab2.2) | | 18) | 20) |
| CDR-H2 | AISGSGGSTYYADSVKG | SGSGGS (SEQ ID NO: | WVSAISGSGGSTY (SEQ |
| (Ab2 and Ab2.2) | (SEQ ID NO: 16) | 19) | ID NO: 48) |
| CDR-H3 | WRNNAFDY (SEQ ID | WRNNAFDY (SEQ ID | AKWRNNAFD (SEQ ID |
| (Ab2 and Ab2.2) | NO: 17) | NO: 17) | NO: 21) |
| CDR-L1 | QGDSLRSYYAS (SEQ | QGDSLRSYYAS (SEQ | SYYASWY (SEQ ID NO: |
| (Ab2 and Ab2.2) | ID NO: 22) | ID NO: 22) | 25) |
| CDR-L2 | GKNNRPS (SEQ ID NO: | GKNNRPS (SEQ ID NO: | LVIYGKNNRP (SEQ ID |
| (Ab2 and Ab2.2) | 23) | 23) | NO: 26) |
| CDR-L3 | NSSPRLKHRVV (SEQ | NSSPRLKHRVV (SEQ | NSSPRLKHRV (SEQ ID |
| (Ab2 and Ab2.2) | ID NO: 24) | ID NO: 24) | NO: 27) |

In some embodiments, an antibody that comprises one or more heavy chain and/or light chain CDR sequences denoted in Table 1 as “Ab1,” binds (e.g., preferentially binds) to WT NPM1. In some embodiments, an antibody that comprises one or more heavy chain and/or light chain CDR sequences denoted in Table 1 as “Ab2,” binds (e.g., preferentially binds) to mutant NPM1 (NPM1c). In some embodiments, an antibody that comprises one or more heavy chain and/or light chain CDR sequences denoted in Table 1 as “Ab2.2,” binds (e.g., preferentially binds) to mutant NPM1 (NPM1c).

In some embodiments, an antibody that binds to NPM1 comprises a heavy chain variable region (V_{H}) sequence and/or light chain variable region (V_{L}) sequence provided in Table 2. In some embodiments, an antibody that binds to NPM1 further comprises a heavy chain constant region (C_{H}) sequence and/or light chain variable region (C_{L}) sequence provided in Table 2. In some embodiments, an antibody that binds to NPM1 comprises a heavy chain sequence and/or light chain sequence provided in Table 2.

TABLE 2
Heavy chain and light chain sequences of anti-NPM1 antibodies

| Antibody Region | Sequence |
|---|---|
| V_{H} (Ab1) | EVQLQQSVAELVRPGASVKLSCTASGFNIKNIFVHWVKQRPEQGLEWIGKIDPA |
| | NDNTKFAPNFQGKAAISADTSSNTAYLQLSSLTSEDTAIYYCASDSSGYDAVDY |
| | WGQGTSVTVSS (SEQ ID NO: 28) |
| V_{L} (Ab1) | DIQMTQSPASLSASVGETVTITCRASESVYTYLAWYQQKQGKSPQLLVYNAKTL |
| | TEGVPSRFSGSGSGTQFSLRINSLQPEDFGSYYCQHHYGTPYTFGSGTKLEIK |
| | (SEQ ID NO: 29) |
| V_{H} (Ab2 and | EVQLLESGGGLVQPGGSLRLSCAASGFTFSSYAMSWVRQAPGKGLEWVSAISGS |
| Ab2.2) | GGSTYYADSVKGRFTISRDNSKNTLYLQMNSLRAEDTAVYYCAKWRNNAFDY |
| | WGQGTLVTVSS (SEQ ID NO: 30) |
| V_{L} (Ab2 and | SSELTQDPAVSVALGQTVRITCQGDSLRSYYASWYQQKPGQAPVLVIYGKNNRP |
| Ab2.2) | SGIPDRFSGSSSGNTASLTITGAQAEDEADYYCNSSPRLKHRVVFGGGTKLTVL |
| | (SEQ ID NO: 31) |
| C_{H} (Ab1/ Ab2) | AKTTAPSVYPLAPVCGDTTGSSVTLGCLVKGYFPEPVTLTWNSGSLSSGVHTFP |
| | AVLQSDLYTLSSSVTVTSSTWPSQSITCNVAHPASSTKVDKKIEPRGPTIKPCPPC |
| | KCPAPNLLGGPSVFIFPPKIKDVLMISLSPIVTCVVVDVSEDDPDVQISWFVNNVE |
| | VHTAQTQTHREDYNSTLRVVSALPIQHQDWMSGKEFKCKVNNKDLPAPIERTIS |
| | KPKGSVRAPQVYVLPPPEEEMTKKQVTLTCMVTDFMPEDIYVEWTNNGKTELN |
| | YKNTEPVLDSDGSYFMYSKLRVEKKNWVERNSYSCSVVHEGLHNHHTTKSFSR |
| | TPGK (SEQ ID NO: 32) |
| C_{H} (Ab2.2) | ASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEK |
| | TISKAKGQPREPQVYTLPPSREEMTKNQVSLTCLVKGFYPSDIAVEWESNGQPEN |
| | NYKTTPPVLDSDGSFFLYSKLTVDKSRWQQGNVFSCSVMHEALHNHYTQKSLS |
| | LSPG (SEQ ID NO: 46) |
| C_{L} (Ab1) | RADAAPTVSIFPPSSEQLTSGGASVVCFLNNFYPKDINVKWKIDGSERQNGVLNS |
| | WTDQDSKDSTYSMSSTLTLTKDEYERHNSYTCEATHKTSTSPIVKSFNRNEC |
| | (SEQ ID NO: 33) |
| C_{L} (Ab2) | GQPKSSPSVTLFPPSSEELETNKATLVCTITDFYPGVVTVDWKVDGTPVTQGMET |
| | TQPSKQSNNKYMASSYLTLTARAWERHSSYSCQVTHEGHTVEKSLSRADCS |
| | (SEQ ID NO: 34) |
| C_{L} (Ab2.2) | GQPKAAPSVTLFPPSSEELQANKATLVCLISDFYPGAVTVAWKADSSPVKAGVE |
| | TTTPSKQSNNKYAASSYLSLTPEQWKSHRSYSCQVTHEGSTVEKTVAPTECS |
| | (SEQ ID NO: 47) |
| Heavy chain (Ab1) | MYRMQLLSCIALSLALVTNSEVQLQQSVAELVRPGASVKLSCTASGFNIKNIFVH |
| | WVKQRPEQGLEWIGKIDPANDNTKFAPNFQGKAAISADTSSNTAYLQLSSLTSE |
| | DTAIYYCASDSSGYDAVDYWGQGTSVTVSSAKTTAPSVYPLAPVCGDTTGSSVT |
| | LGCLVKGYFPEPVTLTWNSGSLSSGVHTFPAVLQSDLYTLSSSVTVTSSTWPSQS |
| | ITCNVAHPASSTKVDKKIEPRGPTIKPCPPCKCPAPNLLGGPSVFIFPPKIKDVLMI |
| | SLSPIVTCVVVDVSEDDPDVQISWFVNNVEVHTAQTQTHREDYNSTLRVVSALPI |
| | QHQDWMSGKEFKCKVNNKDLPAPIERTISKPKGSVRAPQVYVLPPPEEEMTKK |
| | QVTLTCMVTDFMPEDIYVEWTNNGKTELNYKNTEPVLDSDGSYFMYSKLRVEK |
| | KNWVERNSYSCSVVHEGLHNHHTTKSFSRTPGK (SEQ ID NO: 35) |
| Heavy chain (Ab2) | MYRMQLLSCIALSLALVTNSEVQLLESGGGLVQPGGSLRLSCAASGFTFSSYAM |
| | SWVRQAPGKGLEWVSAISGSGGSTYYADSVKGRFTISRDNSKNTLYLQMNSLR |
| | AEDTAVYYCAKWRNNAFDYWGQGTLVTVSSAKTTAPSVYPLAPVCGDTTGSS |
| | VTLGCLVKGYFPEPVTLTWNSGSLSSGVHTFPAVLQSDLYTLSSSVTVTSSTWPS |
| | QSITCNVAHPASSTKVDKKIEPRGPTIKPCPPCKCPAPNLLGGPSVFIFPPKIKDVL |
| | MISLSPIVTCVVVDVSEDDPDVQISWFVNNVEVHTAQTQTHREDYNSTLRVVSA |
| | LPIQHQDWMSGKEFKCKVNNKDLPAPIERTISKPKGSVRAPQVYVLPPPEEEMT |
| | KKQVTLTCMVTDFMPEDIYVEWTNNGKTELNYKNTEPVLDSDGSYFMYSKLR |
| | VEKKNWVERNSYSCSVVHEGLHNHHTTKSFSRTPGK (SEQ ID NO: 36) |
| Heavy chain | MDPKGSLSWRILLFLSLAFELSYGEVQLLESGGGLVQPGGSLRLSCAASGFTFSS |
| (Ab2.2) | YAMSWVRQAPGKGLEWVSAISGSGGSTYYADSVKGRFTISRDNSKNTLYLQMN |
| | SLRAEDTAVYYCAKWRNNAFDYWGQGTLVTVSSASTKGPSVFPLAPSSKSTSG |
| | GTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFFLYS |
| | KLTVDKSRWQQGNVFSCSVMHEALHNHYTQKSLSLSPG (SEQ ID NO: 44) |
| Light chain (Ab1) | MYRMQLLSCIALSLALVTNSDIQMTQSPASLSASVGETVTITCRASESVYTYLAW |
| | YQQKQGKSPQLLVYNAKTLTEGVPSRFSGSGSGTQFSLRINSLQPEDFGSYYCQH |
| | HYGTPYTFGSGTKLEIKRADAAPTVSIFPPSSEQLTSGGASVVCFLNNFYPKDINV |
| | KWKIDGSERQNGVLNSWTDQDSKDSTYSMSSTLTLTKDEYERHNSYTCEATHK |
| | TSTSPIVKSFNRNEC (SEQ ID NO: 37) |
| Light chain (Ab2) | MYRMQLLSCIALSLALVTNSSSELTQDPAVSVALGQTVRITCQGDSLRSYYASW |
| | YQQKPGQAPVLVIYGKNNRPSGIPDRFSGSSSGNTASLTITGAQAEDEADYYCNS |
| | SPRLKHRVVFGGGTKLTVLGQPKSSPSVTLFPPSSEELETNKATLVCTITDFYPGV |
| | VTVDWKVDGTPVTQGMETTQPSKQSNNKYMASSYLTLTARAWERHSSYSCQV |
| | THEGHTVEKSLSRADCS (SEQ ID NO: 38) |
| Light chain | MSVPTQVLGLLLLWLTDARCSSELTQDPAVSVALGQTVRITCQGDSLRSYYAS |
| (Ab2.2) | WYQQKPGQAPVLVIYGKNNRPSGIPDRFSGSSSGNTASLTITGAQAEDEADYYC |
| | NSSPRLKHRVVFGGGTKLTVLGQPKAAPSVTLFPPSSEELQANKATLVCLISDFY |
| | PGAVTVAWKADSSPVKAGVETTTPSKQSNNKYAASSYLSLTPEQWKSHRSYSC |
| | QVTHEGSTVEKTVAPTECS (SEQ ID NO: 45) |

In some embodiments, an antibody that binds to NPM1 may comprise one or more mutations (e.g., amino acid insertions, deletions, or substitutions) relative to a CDR, V_{H}, V_{L}, C_{H}, C_{L}, heavy chain, or light chain sequence provided in Table 1 or Table 2. In some embodiments, an antibody that binds to NPM1 may comprise 1, 2, 3, 4, 5, 6, 7, 8, 9, 10, or more mutations (e.g., amino acid insertions, deletions, or substitutions) relative to a CDR, V_{H}, V_{L}, C_{H}, CL, heavy chain, or light chain sequence provided in Table 1 or Table 2. In some embodiments, an antibody that binds to NPM1 may be at least 60%, at least 70%, at least 80%, at least 90%, at least 95%, or at least 99% identical to a CDR, V_{H}, V_{L}, C_{H}, C_{L}, heavy chain, or light chain sequence provided in Table 1 or Table 2. In some embodiments, the antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the Kabat definition system), a heavy chain complementarity determining region 3 (CDR-H3) of SEQ ID NO: 3 (according to the Kabat definition system), a light chain complementarity determining region 1 (CDR-L1) of SEQ ID NO: 9 (according to the Kabat definition system), a light chain complementarity determining region 2 (CDR-L2) of SEQ ID NO: 10 (according to the Kabat definition system), and a light chain complementarity determining region 3 (CDR-L3) of SEQ ID NO: 11 (according to the Kabat definition system).

In some embodiments, the antibody of the present disclosure comprises a heavy chain complementarity determining region 1 (CDR-H1) of SEQ ID NO: 15 (according to the Kabat definition system), a heavy chain complementarity determining region 2 (CDR-H2) of SEQ ID NO: 16 (according to the Kabat definition system), a heavy chain complementarity determining region 3 (CDR-H3) of SEQ ID NO: 17 (according to the Kabat definition system), a light chain complementarity determining region 1 (CDR-L1) of SEQ ID NO: 22 (according to the Kabat definition system), a light chain complementarity determining region 2 (CDR-L2) of SEQ ID NO: 23 (according to the Kabat definition system), and a light chain complementarity determining region 3 (CDR-L3) of SEQ ID NO: 24 (according to the Kabat definition system).

In some embodiments, the antibody of the present disclosure comprises a heavy chain complementarity determining region 1 (CDR-H1) of SEQ ID NO: 4 (according to the IMGT definition system), a heavy chain complementarity determining region 5 (CDR-H2) of SEQ ID NO: 2 (according to the Chothia definition system), a heavy chain complementarity determining region 3 (CDR-H3) of SEQ ID NO: 3 (according to the Chothia definition system), a light chain complementarity determining region 1 (CDR-L1) of SEQ ID NO: 9 (according to the Chothia definition system), a light chain complementarity determining region 2 (CDR-L2) of SEQ ID NO: 10 (according to the Chothia definition system), and a light chain complementarity determining region 3 (CDR-L3) of SEQ ID NO: 11 (according to the Chothia definition system).

In some embodiments, the antibody of the present disclosure comprises a heavy chain complementarity determining region 1 (CDR-H1) of SEQ ID NO: 18 (according to the Chothia definition system), a heavy chain complementarity determining region 2 (CDR-H2) of SEQ ID NO: 19 (according to the Chothia definition system), a heavy chain complementarity determining region 3 (CDR-H3) of SEQ ID NO: 17 (according to the Chothia definition system), a light chain complementarity determining region 1 (CDR-L1) of SEQ ID NO: 22 (according to the Chothia definition system), a light chain complementarity determining region 2 (CDR-L2) of SEQ ID NO: 23 (according to the Chothia definition system), and a light chain complementarity determining region 3 (CDR-L3) of SEQ ID NO: 24 (according to the Chothia definition system).

In some embodiments, the antibody of the present disclosure comprises a heavy chain complementarity determining region 1 (CDR-H1) of SEQ ID NO: 6 (according to the IMGT definition system), a heavy chain complementarity determining region 2 (CDR-H2) of SEQ ID NO: 7 (according to the Contact definition system), a heavy chain complementarity determining region 3 (CDR-H3) of SEQ ID NO: 8 (according to the Contact definition system), a light chain complementarity determining region 1 (CDR-L1) of SEQ ID NO: 12 (according to the Contact definition system), a light chain complementarity determining region 2 (CDR-L2) of SEQ ID NO: 13 (according to the Contact definition system), and a light chain complementarity determining region 3 (CDR-L3) of SEQ ID NO: 14 (according to the Contact definition system).

In some embodiments, the antibody of the present disclosure comprises a heavy chain complementarity determining region 1 (CDR-H1) of SEQ ID NO: 20 (according to the Contact definition system), a heavy chain complementarity determining region 2 (CDR-H2) of SEQ ID NO: 48 (according to the Contact definition system), a heavy chain complementarity determining region 3 (CDR-H3) of SEQ ID NO: 21 (according to the Contact definition system), a light chain complementarity determining region 1 (CDR-L1) of SEQ ID NO: 25 (according to the Contact definition system), a light chain complementarity determining region 2 (CDR-L2) of SEQ ID NO: 26 (according to the Contact definition system), and a light chain complementarity determining region 3 (CDR-L3) of SEQ ID NO: 27 (according to the Contact definition system).

In some embodiments, the antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 28. Alternatively or in addition (e.g., in addition), the antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 29.

In some embodiments, the antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 30. Alternatively or in addition (e.g., in addition), the antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 31.

In some embodiments, the antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 28. Alternatively or in addition (e.g., in addition), in some embodiments, the antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 29.

In some embodiments, the antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 30. Alternatively or in addition (e.g., in addition), in some embodiments, the antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 31.

In some embodiments, the antibody of the present disclosure comprises a VH comprising the amino acid sequence of SEQ ID NO: 28. Alternatively or in addition (e.g., in addition), in some embodiments, the antibody of the present disclosure comprises a VL comprising the amino acid sequence of SEQ ID NO: 29.

In some embodiments, the antibody of the present disclosure comprises a VH comprising the amino acid sequence of SEQ ID NO: 30. Alternatively or in addition (e.g., in addition), in some embodiments, the antibody of the present disclosure comprises a VL comprising the amino acid sequence of SEQ ID NO: 31.

In some embodiments, the antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 35. In some embodiments, the antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 35. Alternatively or in addition (e.g., in addition), the antibody of the present disclosure comprises a light chain comprising an amino acid sequence least 75% (e.g., 75%, 80%, 85%, 90%, 95%, 98%, or 99%) identical to the amino acid sequence of SEQ ID NO: 37. Alternatively or in addition (e.g., in addition), the antibody of the present disclosure comprises a light chain comprising an amino acid sequence least 75% (e.g., 75%, 80%, 85%, 90%, 95%, 98%, or 99%) identical to the amino acid sequence of SEQ ID NO: 37.

In some embodiments, the antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 36. In some embodiments, the antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 36. Alternatively or in addition (e.g., in addition), the antibody of the present disclosure comprises a light chain comprising an amino acid sequence least 75% (e.g., 75%, 80%, 85%, 90%, 95%, 98%, or 99%) identical to the amino acid sequence of SEQ ID NO: 38. Alternatively or in addition (e.g., in addition), the antibody of the present disclosure comprises a light chain comprising an amino acid sequence least 75% (e.g., 75%, 80%, 85%, 90%, 95%, 98%, or 99%) identical to the amino acid sequence of SEQ ID NO: 38.

In some embodiments, the antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 44. In some embodiments, the antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 44. Alternatively or in addition (e.g., in addition), the antibody of the present disclosure comprises a light chain comprising an amino acid sequence least 75% (e.g., 75%, 80%, 85%, 90%, 95%, 98%, or 99%) identical to the amino acid sequence of SEQ ID NO: 45. Alternatively or in addition (e.g., in addition), the antibody of the present disclosure comprises a light chain comprising an amino acid sequence least 75% (e.g., 75%, 80%, 85%, 90%, 95%, 98%, or 99%) identical to the amino acid sequence of SEQ ID NO: 45.

In some embodiments, the antibody of the present disclosure comprises a heavy chain comprising the amino acid sequence of SEQ ID NO: 35. In some embodiments, the antibody of the present disclosure comprises a heavy chain comprising the amino acid sequence of SEQ ID NO: 35. Alternatively or in addition (e.g., in addition), the antibody of the present disclosure comprises a light chain comprising the amino acid sequence of SEQ ID NO: 37. Alternatively or in addition (e.g., in addition), the antibody of the present disclosure comprises a light chain comprising the amino acid sequence of SEQ ID NO: 37.

In some embodiments, the antibody of the present disclosure comprises a heavy chain comprising the amino acid sequence of SEQ ID NO: 36. In some embodiments, the antibody of the present disclosure comprises a heavy chain comprising the amino acid sequence of SEQ ID NO: 36. Alternatively or in addition (e.g., in addition), the antibody of the present disclosure comprises a light chain comprising the amino acid sequence of SEQ ID NO: 38. Alternatively or in addition (e.g., in addition), the antibody of the present disclosure comprises a light chain comprising the amino acid sequence of SEQ ID NO: 38.

In some embodiments, the antibody of the present disclosure comprises a heavy chain comprising the amino acid sequence of SEQ ID NO: 44. In some embodiments, the antibody of the present disclosure comprises a heavy chain comprising the amino acid sequence of SEQ ID NO: 44. Alternatively or in addition (e.g., in addition), the antibody of the present disclosure comprises a light chain comprising the amino acid sequence of SEQ ID NO: 45. Alternatively or in addition (e.g., in addition), the antibody of the present disclosure comprises a light chain comprising the amino acid sequence of SEQ ID NO: 45.

### Preparation of Anti-NPM1 Antibodies

Antibodies capable of binding NPM1 as described herein can be made by any method known in the art. See, for example, Harlow and Lane, (1998) Antibodies: A Laboratory Manual, Cold Spring Harbor Laboratory, New York. In some examples, an anti-NPM1 antibody is prepared by recombinant technology.

For example, hybridoma cell lines can be prepared from lymphocytes and immortalized myeloma cells expressing an antibody described herein using techniques that are broadly known in the art (see, e.g., Kohler and Milstein, (1975) Nature 256:495-497; or Buck, et al., (1982) In Vitro, 18:377-381). Typically, the generation of a hybridoma involves fusing a myeloma cell with a lymphoid cell using a fusogen (e.g., polyethylene glycol or an electrical current) to produce an immortalized cell line capable of producing and secreting the desired antibody. Hybridomas may be cultured in culture media under conditions that are known in the art.

Alternately, nucleic acids encoding the heavy and light chain of an anti-NPM1 antibody as described herein can be cloned using recombinant technology known in the art into one or more vectors, wherein each nucleotide sequence is operably linked to a suitable promoter (e.g., a constitutive or inducible promotor, as known in the art). The vectors may be expression vectors, from which polypeptide sequences for an anti-NPM1 antibody can be expressed in a particular cell type. The one or more expression vectors comprise one or more promoters for driving the expression of an operably linked gene (typically 3′ to the promotor sequence). Each of nucleotide sequence encoding the heavy chain and light chain may be operably linked to different promoters. Alternatively, the nucleotide sequences encoding the heavy chain and the light chain may be operably linked to the same promoters (e.g., within the same expression vector). The selection of expression vector(s) and/or promoter(s) depends on the type of host cells for use in producing the antibodies. When necessary, an internal ribosomal entry site (IRES) can be inserted between the heavy chain and light chain encoding sequences to enhance recombinant expression. Expression vectors encoding an anti-NPM1 antibody may be inserted into a cell for expression using any technique known in the art (e.g., chemical transfection, viral transfection). The cell may be a cell which can be cultured using techniques known in the art. The cell may be any cell type capable of expressing heterologous antibody sequences, such as, for example, a human cell, a murine cell, a rat cell, a non-human primate cell, or an insect cell that can be cultured in vitro. After culturing such a cell under conditions sufficient for expression of the antibody (e.g., under temperature and growth media conditions sufficient for expression), the anti-NPM1 antibody may be isolated from either the culture media (if the antibody is secreted) or from the cells following lysis. Techniques for isolating antibodies from cells and/or culture media are generally known in the art, but typically include affinity chromatography.

### Anti-NPM1 Antibody Conjugates

The present disclosure further provides molecular conjugates that comprise an anti-NPM1 antibody described herein (e.g., an antibody that binds to WT NPM1 or mutant NPM1) which is linked through a chemical linker to an agent.

In some embodiments, a conjugate described herein comprises an anti-NPM1 antibody that is linked to a drug. A conjugate comprising an anti-NPM1 antibody that is conjugated to a drug may be referred to as an “ADC.” In some embodiments, the drug is a small molecule. The term “small molecule” refers to molecules, whether naturally occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible.

In some embodiments, the drug is a cytotoxic drug (e.g., a cytotoxic small molecule). In some embodiments, a drug is a cytostatic drug (e.g., a cytostatic small molecule). In some embodiments, the drug is a chemotherapeutic drug. Non-limiting examples of drugs suitable for use in the ADCs described herein include auristatin E, auristatin F, monomethyl auristatin D (MMAD), monomethyl auristatin F (MMAF), monomethyl auristatin E (MMAE), actinomycin, actinomycin X2, α-amanitin, β-amanitin, γ-amanitin, ε-amanitin, aeroplysinin, aldoxorubicin, agrochelin, ansatrienin, ansamitocin P-3, aphidicolin, apoptolidin, L-asparaginase, azacitidine, bafilomycin A1, bafilomycin B1, bafilomycin B2, bafilomycin C1, bafilomycin C2, bafilomycin D, bafilomycin E, calicheamicin, campathecin, chaetocin, chaetoglobosin, chlamydocin, cinerubin B, cladribine, colchicine, combretastatin A1, combretastatin A4, cordycepin, cryptophycin, cucurbitacin B, cucurbitacin E, curvulin, cyclopamine, cyclophosphamide, cytarabine, dactinomycin, daunorubicin, decitabine, dexamethasone, dolastatin 10, dolastatin 15, duocarmycin SA, duocarmycin TM, duocarmycin MA, duocarmycin DM, doxorubicin, englerin A, epothilone A, epothilone B, epothilone C, etoposide, fludarabine, fumagillin, geldanamycin, tanespimycin (17-AAG), glucopiericidin A, gramicidin A, herboxidiene, 9-hydroxyellipticine, hydroxyurea, hygrolidin, hypothemycin, idarubicin, ilimaquinone, isatropolone A, isofistularin-3, ixabepilone, JW55, lactacystin, luisol A, maytansinol, mertansine (DM1), maytansine DM3, ravtansine (DM4), maytansinoid AP-3, mechercharmycin A, mensacarcin, methotrexate, 6-mercaptopurine, microcolin B, microcystin LR, mitoxantrone, muscotoxin A, myoseverin, mytoxin B, nelarabine, nemorubicin, nocuolin A, okilactomycin, oligomycin A, oligomycin B, paclitaxel, larotaxel, milataxel, ortataxel, tesetaxel, phallacidin, phalloidin, phytosphingosine, piericidin A, pironetin, podophyllotoxin, polyketomycin, prednisone, pseudolaric acid B, pseurotin A, puwainaphycin F, pyrrolobenzodiazepine, quinaldopeptin, rachelmycin, rebeccamycin, Ro 5-3335, safracin B, sandramycin, sanguinarine, saporin, sinefungin, taltobulin, telomestatin, 6-thioguanine, thiocolchicine, tolytoxin, tripolin A, triptolide, tubastatin A, tubulysin A, tubulysin M, tubulysin IM-1, tubulysin IM-2, tubulysin IM-3, venetoclax, and vincristine. An ADC described herein may comprise another drug (e.g., a chemotherapeutic drug) that is known in the art to be cytotoxic or cytostatic toward a particular cell type (e.g., a cancer cell).

In some embodiments, a conjugate described herein comprises an anti-NPM1 antibody that is linked to an imaging agent. An imaging agent may be a small molecule. An imaging agent has one or more properties by which presence of the agent may be detected. For example, an imaging agent described herein may be a luminescent imaging agent (e.g., luciferase) or a fluorescent imaging agent (e.g., fluorescein sodium, methylene blue, indocyanine green (ICG)), which can be detected my measuring the emission of light at one or more specific wavelengths using techniques generally known in the art. Alternately, an imaging agent may be an agent that is detectable using magnetic resonance imaging (MRI), such as, but not limited to, gadolinium-diethylenetriamine (Gd-DTPA).

In some embodiments, an anti-NPM1 antibody described herein is conjugated to more than one molecule of an agent (e.g., a drug, an imaging agent), i.e., the antibody and the agent are conjugated together at a ratio greater than 1:1. In some embodiments, the ratio between the antibody and the agent is between 1:1 and 1:10. In some embodiments, the ratio between the antibody and the agent is 1:2, 1:3, 1:4, 1:5, 1:6, 1:7, 1:8, 1:9, or 1:10.

In some embodiments, an anti-NPM1 antibody described herein is conjugated (linked) to an agent using a chemical linker known in the art. As used herein, the terms “conjugated,” “linked,” and “attached” mean that two molecules are associated, preferably through a covalent bond or with sufficient affinity that the therapeutic or diagnostic benefit of the association between the two entities is realized.

In some embodiments, a linker is a cleavable linker. As used herein, a cleavable linker is capable of releasing a conjugated moiety in response to a stimulus. In some embodiments, the stimulus is a physiological stimulus. Non-limiting examples of stimuli include the presence of an enzyme, acidic conditions, basic conditions, or reducing conditions. For example, cleavable linkers include peptide linkers, β-glucuronide linkers, glutathione-sensitive linkers (or disulfide linkers) and pH-sensitive linkers. In some embodiments, a pH-sensitive linker is cleaved at a pH between 5.0 and 6.5 or between a pH of 4.5 and 5.0. In some embodiments, a pH-sensitive linker is not cleaved when the pH is between 7 and 7.5. In some embodiments, a pH-sensitive linker is not cleaved when the pH is between 7.3 and 7.5. In some embodiments, a cleavable linker is a protease-sensitive linker. Examples of cleavable linkers include N-succinimidyl 4-(2-pyridyldithio)pentanoate (SPP), N-succinimidyl 3-(2-pyridyldithio)butanoate (SPDB), Sulfo-SPDB, valine-citrulline dipeptide (Val-Cit), acetyl butyrate, CL2A, maleimidocaproyl (MC), and Mal-EBE-Mal. See, e.g., Donaghy, mAbs. 2016 May-June; 8(4):659-71, incorporated herein by reference.

In some embodiments, a linker is non-cleavable. In some embodiments, a non-cleavable linker is a linker that is not cleaved within systemic circulation in a subject. In some embodiments, a non-cleavable linker is a linker that is resistant to protease cleavage. Non-cleavable linkers include N-succinimidyl 4-(N-maleimidomethyl)cyclohexane-1-carboxylate (SMCC), maleimidomethyl cyclohexane-1-carboxylate (MCC) and MC-VC-PAB.

Any of the conjugates described herein may be synthesized using methods known in the art. See, e.g., Yao et al., Int J Mol Sci. 2016 Feb. 2; 17 (2).

The ADCs comprising an anti-NPM1 antibody conjugated to a drug are advantageous to use therapeutically, in part because the drugs (e.g., chemotherapeutic drugs) are toxic and can be targeted to particular cell types expressing cell surface NPM1 (e.g., NPM1-expressing cancer cells). By conjugating the drug to the anti-NPM1 antibody, the toxicity of the ADC may be reduced by at least 20%, at least 30%, at least 40%, at least 50%, at least 60%, at least 70%, at least 80%, at least 90%, at least 99%, compared to the drug in its free from. Conjugates comprising an anti-NPM1 antibody conjugated to an imaging agent are advantageous as they are specific for particular cells and tissues expressing cell surface NPM1 (e.g., NPM1-expressing cancer cells), enabling greater specificity of the imaging agent for the cell or tissue than could be achieved by the free imaging agent. Such conjugates are useful for detecting the presence and abundance of cells and tissues expressing cell surface NPM1 (e.g., NPM1-expressing cancer cells) and may be administered, for example, prior to, during, or after any course of treatment.

In some embodiments, the agent to which an anti-NPM1 antibody is linked is a radioisotope. In some embodiments, the radioisotope is a radioisotope that is useful for the treatment of a cancer (i.e., a therapeutic radioisotope), such as, for example a radioisotope that is useful for the treatment of acute myeloid leukemia (AML), acute promyeloid leukemia (APL), acute lymphoblastic leukemia (ALL), non-Hodgkin's lymphoma, or myelodysplastic syndrome (MDS). Radioisotopes that are useful for treatment of cancer are well known in the art and include, but are not limited to, Iodine-131 (¹³¹I), Rhenium-188 (¹⁸⁸Re), Yttrium-90 (⁹⁰Y), Bismuth-213 (²¹³Bi), and Actinium-225 (²²⁵Ac). Examples of radioisotope-linked antibodies and techniques for producing such antibodies are also well known in the art (see, e.g., Rosenblat T L, et al. “Sequential cytarabine and alpha-particle immunotherapy with bismuth-213-lintuzumab (HuM195) for acute myeloid leukemia”. Clin Cancer Res. 2010 Nov. 1; 16(21):5303-11, which is incorporated herein by reference). Antibodies comprising a radioisotope are advantageous to use therapeutically, in part because the radioisotopes are toxic and can be targeted to particular cell types expressing cell surface NPM1 (e.g., NPM1-expressing cancer cells). By conjugating a radioisotope to an anti-NPM1 antibody, the toxicity of the radioisotope may be reduced by at least 20%, at least 30%, at least 40%, at least 50%, at least 60%, at least 70%, at least 80%, at least 90%, at least 99%, compared to the radioisotope in its free from.

### Compositions

The present disclosure further provides compositions comprising an antibody that binds to NPM1 provided herein (e.g., an antibody that binds to WT NPM1 or mutant NPM1). In some embodiments, the pharmaceutical composition further comprises a pharmaceutically acceptable carrier. “Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” may be a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for administration to a subject, each unit containing a predetermined quantity of active material calculated to produce the desired therapeutic effect in association with the required diluent; i.e., carrier, or vehicle.

The formulation of the pharmaceutical composition may dependent upon the route of administration to a subject. Injectable preparations suitable for parenteral administration or intraperitoneal, intratumoral, peritumoral, intralesional or perilesional administration include, for example, sterile injectable aqueous or oleaginous suspensions and may be formulated according to the known art using suitable dispersing or wetting agents and suspending agents. The injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

In some embodiments, compositions (e.g., pharmaceutical compositions administered to a subject) provided herein must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

### Administration of Anti-NPM1 Conjugates

The present disclosure further provides for the administration of an anti-NPM1 antibody described herein, a conjugate comprising an anti-NPM1 antibody described herein, or a composition thereof (e.g., a pharmaceutical composition) to a subject. In some embodiments, a method is provided for treating a disease associated with cell surface expression of NPM1 (e.g., a NPM1-expressing cancer) by administering an anti-NPM1 antibody described herein, a conjugate comprising an anti-NPM1 antibody described herein, or a composition thereof (e.g., a pharmaceutical composition) to a subject. In some embodiments, a method is provided for evaluating the presence of a disease associated with cell surface expression of NPM1 (e.g., a NPM1-expressing cancer) by administering an anti-NPM1 antibody described herein, a conjugate comprising an anti-NPM1 antibody described herein, or a composition thereof (e.g., a pharmaceutical composition) to a subject.

In some aspects, the present disclosure provides a method for treating a NPM1-expressing cancer in a subject, comprising administering an effective amount of an anti-NPM1 antibody described herein, a conjugate comprising an anti-NPM1 antibody that is conjugated to an agent (e.g., a drug, a radioisotope) described herein, or a composition thereof (e.g., a pharmaceutical composition), to a subject in need thereof.

As used herein, the terms “administer,” “administering,” or “administration” refer to implanting, absorbing, ingesting, injecting, inhaling, or otherwise introducing an agent described herein (e.g., an antibody, a conjugate), or a composition thereof (e.g., a pharmaceutical composition), in or on a subject. As used herein, the term “treatment,” “treat,” and “treating” refers to the application or administration of an agent described herein (e.g., an antibody, a conjugate), or a composition thereof (e.g., a pharmaceutical composition), to a subject in need thereof for the purpose of reducing the severity of a disease (e.g., a cancer) in the subject. A “subject in need thereof” refers to an individual that has a disease, a symptom of the disease, or a predisposition toward the disease. A method for treating a disease may encompass administering to a subject an agent described herein (e.g., an antibody, a conjugate), or a composition thereof (e.g., a pharmaceutical composition) with the intention to cure, heal, alleviate, relieve, alter, remedy, ameliorate, improve, or affect the disease, a symptom of the disease, or predisposition toward the disease in the subject. A method for treating a disease may encompass prophylaxis, wherein an agent is administered to the subject for the purpose of preventing development of the disease, for example, in a subject that is not known to have the disease, but may develop or be at risk of developing the disease in the future.

As used herein, a “therapeutically effective amount” or “effective amount” refers to the amount of an agent (e.g., an antibody or conjugate described herein) that is sufficient to elicit the desired biological response in the subject, for example, alleviating one or more symptoms of the disease (e.g., a cancer). A therapeutically effective amount may be an amount that is either administered to the subject alone or in combination with one or more other agents. Effective amounts vary, as recognized by those skilled in the art, depending on such factors as the desired biological endpoint, the pharmacokinetics of the administered agent, the particular condition or disease being treated, the severity of the condition or disease, the individual parameters of the subject, including age, physical condition, size, gender and weight, the duration of the treatment, the nature of any other concurrent therapy, the specific route of administration, and like factors that are within the knowledge and expertise of the health practitioner to determine. These factors are well known to those of ordinary skill in the art and can be addressed with no more than routine experimentation. It is generally preferred that a maximum dose of the individual agents described herein (e.g., an antibody or conjugate described herein) or any combinations thereof to be used is at most the highest dose that can be safely administered to the subject according to sound medical judgment. Preferably, an effective dose is lower than the highest dose that can be safely administered to the subject. It will be understood by those of ordinary skill in the art, however, that a subject or health practitioner may select a lower dose (e.g., the minimum effective dose) in order to mitigate any potential risks of treatment, such as side effects of the treatment.

In some embodiments, for an adult subject of normal weight, doses ranging from about 0.01 to 1000 mg/kg of an agent (e.g., an antibody or conjugate described herein) may be administered. In some embodiments, the dose is between 1 to 200 mg. The particular dosage regimen, i.e., the dose, timing, and repetition, will depend on the particular subject and that subject's medical history, as well as the properties of the agent (such as the pharmacokinetics of the agent) and other consideration well known in the art.

Treating a disease (e.g., cancer) may include delaying the development or progression of the disease or reducing disease severity. Treating the disease does not necessarily require curative results. As used herein, “delaying” the development of a disease means to defer, hinder, slow, retard, stabilize, and/or postpone progression of the disease in a subject. Delaying the progression of a disease may include delaying or preventing the spread of a disease occurring in a subject, such as, for example, delaying or preventing the metastasis of a cancer occurring in a subject to one or more organs or tissues not yet affected by the cancer. This delay can be of varying lengths of time, depending on the history of the disease and/or individuals being treated. A method that delays the development of a disease, or delays the onset of the disease, is a method that reduces probability of developing one or more symptoms of the disease in a given time frame and/or reduces extent of the symptoms in a given time frame, as compared to the absence of such a method. Comparisons are typically based on clinical studies, using a number of subjects sufficient to give a statistically significant result.

The “development” or “progression” of a disease (e.g., a cancer) refers to initial manifestations and/or ensuing progression of the disease in a subject. Development of a disease can be detectable and assessed using standard clinical techniques as well known in the art. However, development also refers to progression that may be undetectable. For purpose of this disclosure, development or progression may refer to the development or progression of symptoms of a disease. The term “development” includes the occurrence, recurrence, and onset of a disease. As used herein “onset” or “occurrence” of a disease includes the initial onset of a disease, as well as recurrence of the disease (i.e., in a subject who has had the disease previously).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or a non-human animal. In some embodiments, the non-human animal is a mammal (e.g., rodent, e.g., mouse or rat), a primate (e.g., cynomolgus monkey or rhesus monkey), a commercially relevant mammal (e.g., cattle, pig, horse, sheep, goat, cat, or dog), or a bird (e.g., commercially relevant bird, such as chicken, duck, goose, or turkey). The non-human animal may be a male or female at any stage of development and may be a juvenile animal or an adult animal. The non-human animal may be a transgenic animal or genetically engineered animal.

In some embodiments, the subject is a companion animal (e.g., a pet or service animal). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include rodents (e.g., rats, mice, guinea pigs, and hamsters), rabbits, or non-human primates.

In some embodiments, the subject has, is suspected of having, or is at risk for a NPM1-expressing cancer. The term “NPM1-expressing cancer” refers to a cancer that is characterized by expression of NPM1 (e.g., enhanced expression of NPM1, as compared to a noncancerous cell), which may be further characterized by a change in the subcellular localization of NPM1, such as, for example, an increase of NPM1 present on the cell surface. An NPM1-expressing cancer may express WT NPM1 and/or mutant NPM1. A NPM1-expressing cancer may express WT NPM1 and/or mutant NPM1 on the cell surface. A NPM1-expressing cancer may also refer to a cancer which is not initially characterized by expression of NPM1, that expresses NPM1 (e.g., cell surface WT NPM1 and/or mutant NPM1) in response to administration of or treatment with an agent (e.g., a chemotherapeutic drug). In some embodiments, a NPM1-expressing cancer is a solid (tissue) or liquid (biological fluid, e.g., blood) cancer. In some embodiments, a NPM1-expressing cancer is a cancer selected from a hematological cancer, a lung cancer, a breast cancer, a brain cancer, a gastrointestinal cancer, a liver cancer, a kidney cancer, a bladder cancer, a pancreatic cancer, an ovarian cancer, a testicular cancer, a prostate cancer, an endometrial cancer, a muscle cancer, a bone cancer, a neuroendocrine cancer, a connective tissue cancer, a head or neck cancer, or a skin cancer. In some embodiments, a NPM1-expressing cancer is acute myeloid leukemia (AML), acute promyeloid leukemia (APL), acute lymphoblastic leukemia (ALL), non-Hodgkin's lymphoma, and myelodysplastic syndrome (MDS). In some embodiments, a NPM1-expressing cancer is a metastatic cancer. In some embodiments, a NPM1-expressing cancer is a therapy-related cancer or a secondary malignancy. A therapy-related cancer or a secondary malignancy may be a cancer, such as a leukemia, carcinoma, or a lymphoma, resulting from a previous treatment with a chemotherapeutic or a radioisotope. In some embodiments, a NPM1-expressing cancer is therapy-related AML (t-AML) or a secondary malignancy of non-Hodgkin's lymphoma.

In some embodiments, the subject has been treated previously for a NPM1-expressing cancer. In some embodiments, the subject has a NPM1-expressing cancer that is resistant toward one or more treatments (e.g., treatment with one or more chemotherapeutic drugs). A NPM1-expressing cancer is said to be resistant toward a treatment (e.g., treatment with one or more chemotherapeutic drugs) if the treatment cannot effectively kill and/or inactivate cells of the cancer, for example, due to genetic and/or epigenetic changes occurring in the cancer that result in inactivation and/or efflux of one or more drugs of the treatment, or inhibition of one or more downstream effects of the treatment, especially if the treatment was previously effective for killing and/or inactivating cells of the cancer. In some embodiments, a NPM1-expressing cancer that is resistant toward one or more treatments (e.g., treatment with one or more chemotherapeutic drugs) expresses NPM1 on the surface of cancer cells as a result of a previous treatment (e.g., a previous treatment with one or more chemotherapeutic drugs) to which the cancer is or has become resistant to. In some embodiments, the level of NPM1 on the surface of a NPM1-expressing cancer that is resistant toward one or more treatments (e.g., treatment with one or more chemotherapeutic drugs) is increased as a result of a previous treatment (e.g., a previous treatment with one or more chemotherapeutic drugs) to which the cancer is or has become resistant to.

Conventional methods, known to those of ordinary skill in the art of medicine, can be used to administer the antibody, conjugate, or composition thereof (e.g., a pharmaceutical composition) to the subject, depending upon the type of disease (e.g., cancer) to be treated or the site of the disease (e.g., cancer). The antibody, conjugate, or composition thereof (e.g., a pharmaceutical composition) can be administered systemically (i.e., throughout the body) or locally (i.e., to one or more specific organs, tissues, or locations in the body). The antibody, conjugate, or composition thereof (e.g., a pharmaceutical composition) can be administered via any conventional route, e.g., administered orally, parenterally, by inhalation spray, topically, rectally, nasally, buccally, vaginally, intraperitoneal, or via an implanted reservoir. The term “parenteral” as used herein includes subcutaneous, intracutaneous, intravenous, intramuscular, intraarticular, intraarterial, intrasynovial, intrasternal, intraperitoneal, intrathecal, intralesional, and intracranial injection or infusion techniques. In some embodiments, the antibody, conjugate, or composition thereof (e.g., a pharmaceutical composition) is administered via intravenous injection or infusion. In addition, the antibody, conjugate, or composition thereof (e.g., a pharmaceutical composition) can be administered to the subject via injectable depot routes of administration such as using 1-, 3-, or 6-month depot injectable or biodegradable materials and methods. In some embodiments, the antibody, conjugate, or composition thereof (e.g., a pharmaceutical composition) is administered via injection. In some embodiments, the injection is intravenous injection or intratumoral injection.

In some embodiments, the administration occurs more than once. In some embodiments, the administration occurs once per day, once per 2 days, once per 3 days, once per 4 days, once per 5 days, once per 6 days, once per week, once per 2 weeks, once per 3 weeks, once per month, once per 2 months, once per 3 months, once per 4 months, once per 6 months, once per 7 months, once per 8 months, once per 9 months, once per 10 months, once per 11 months, or once per year.

In some embodiments, the administration results in reduced growth of NPM1-expressing cancer cells of the subject, as compared to growth of NPM1-expressing cancer cells of the subject in the absence of the administration. As used herein, “reduced growth” refers to a reduction in the rate of cell division (mitosis) occurring in NPM1-expressing cancer cells of the subject. In some embodiments, the administration results in reduced growth of NPM1-expressing cancer cells of the subject by up to 5%, up to 10%, up to 15%, up to 20%, up to 25%, up to 30%, up to 35%, up to 40%, up to 45%, up to 50%, up to 60%, up to 70%, up to 80%, up to 90%, up to 100%, up to 2-fold, up to 3-fold, up to 4-fold, up to 5-fold, up to 6-fold, up to 7-fold, up to 8-fold, up to 9-fold, or up to 10-fold, as compared to growth of NPM1-expressing cancer cells in the absence of the administration.

In some embodiments, the administration results in increased cell death of NPM1-expressing cancer cells of the subject, as compared to cell death of NPM1-expressing cancer cells of the subject in the absence of the administration. As used herein, “cell death” refers to an increase in the rate of cell death occurring in NPM1-expressing cancer cells of the subject via any pathway by which cells cease to be viable, such as, but not limited to, apoptosis, autophagy, necrosis, and entosis. In some embodiments, the administration results in increased cell death of NPM1-expressing cancer cells of the subject by up to 5%, up to 10%, up to 15%, up to 20%, up to 25%, up to 30%, up to 35%, up to 40%, up to 45%, up to 50%, up to 60%, up to 70%, up to 80%, up to 90%, up to 100%, up to 2-fold, up to 3-fold, up to 4-fold, up to 5-fold, up to 6-fold, up to 7-fold, up to 8-fold, up to 9-fold, or up to 10-fold, as compared to cell death of NPM1-expressing cancer cells of the subject in the absence of the administration.

In some aspects, the present disclosure further provides a method for evaluating the presence of (detecting) a NPM1-expressing cancer in a subject, comprising administering an effective amount of an anti-NPM1 antibody described herein, a conjugate comprising an anti-NPM1 antibody that is conjugated to an imaging agent described herein, or a composition thereof (e.g., a pharmaceutical composition), to a subject in need thereof.

In some embodiments, such a method comprises administering to a subject in need thereof an effective dose of an anti-NPM1 antibody described herein, a conjugate between an anti-NPM1 antibody described herein and an imaging agent, or a composition thereof (e.g., a pharmaceutical composition), imaging the antibody or conjugate, and determining the presence of a NPM1-expressing cancer in the subject based on the level and position of the antibody or conjugate imaged in the subject. The effective amount will vary, as recognized by those skilled in the art, depending on the particular condition to be detected, the severity of the condition, the individual subject parameters including age, physical condition, size, gender and weight, the duration of the detection, the specific route of administration and like factors within the knowledge and expertise of the health practitioner. In some embodiments, the NPM1-expressing cancer is a cancer in which WT NPM1 and/or mutant NPM1 is expressed on the surface of cancer cells. In some embodiments, a NPM1-expressing cancer is a solid (tissue) or liquid (biological fluid, e.g., blood) cancer. In some embodiments, a NPM1-expressing cancer is a cancer selected from a hematological cancer, a lung cancer, a breast cancer, a brain cancer, a gastrointestinal cancer, a liver cancer, a kidney cancer, a bladder cancer, a pancreatic cancer, an ovarian cancer, a testicular cancer, a prostate cancer, an endometrial cancer, a muscle cancer, a bone cancer, a neuroendocrine cancer, a connective tissue cancer, a head or neck cancer, or a skin cancer. In some embodiments, a NPM1-expressing cancer is acute myeloid leukemia (AML), acute promyeloid leukemia (APL), acute lymphoblastic leukemia (ALL), non-Hodgkin's lymphoma, and myelodysplastic syndrome (MDS). In some embodiments, a NPM1-expressing cancer is a metastatic cancer. In some embodiments, a NPM1-expressing cancer is a therapy-related cancer or a secondary malignancy. In some embodiments, a NPM1-expressing cancer is therapy-related AML (t-AML) or a secondary malignancy of non-Hodgkin's lymphoma.

In some embodiments, the imaging conducted is luminescent imaging or fluorescent imaging. The method of imaging may be selected based on the imaging agent administered to the subject, for example, the type of imaging agent conjugated to or binding to an anti-NPM1 antibody administered to the subject. In some embodiments, the imaging is magnetic resonance imaging (MRI).

In some embodiments, a method of evaluating the presence of (detecting) an NPM1-expressing cancer in a subject further comprises administering an effective amount of an anti-NPM1 antibody, a conjugate comprising an anti-NPM1 antibody and an agent (e.g., a drug, a radioisotope) described herein, or a composition thereof (e.g., a pharmaceutical composition) to the subject, for example, if a NPM1-expressing cancer is determined to be present in the subject. The effective amount of an anti-NPM1 antibody, a conjugate comprising an anti-NPM1 antibody and an agent (e.g., a drug, a radioisotope), or a composition thereof (e.g., a pharmaceutical composition) to be administered to the subject may depend on a variety of factors that are within the knowledge and expertise of the health practitioner to determine, including the amount and location of the NPM1-expressing cancer that is determined to be present in the subject.

In further aspects, the present disclosure provides ex vivo methods for evaluating the presence of (detecting) a NPM1-expressing cancer in a subject. For instance, some aspects of the present disclosure relate to a method for evaluating the presence of (detecting) a NPM1-expressing cancer in a subject, comprising collecting a biological sample from a subject in need thereof (or providing a biological sample previously obtained from such a subject), contacting the biological sample with an anti-NPM1 antibody or a conjugate between an anti-NPM1 antibody and an imaging agent described herein, analyzing binding between the antibody or conjugate and NPM1-expressing cancer cells in the biological sample, and determining the presence of a NPM1-expressing cancer in the subject, based on the level of binding between the conjugate and NPM1-expressing cancer cells analyzed in the sample.

In some embodiments, the NPM1-expressing cancer is a cancer in which WT NPM1 and/or mutant NPM1 is expressed on the surface of cancer cells. In some embodiments, a NPM1-expressing cancer is a solid (tissue) or liquid (biological fluid, e.g., blood) cancer. In some embodiments, a NPM1-expressing cancer is a cancer selected from a hematological cancer, a lung cancer, a breast cancer, a brain cancer, a gastrointestinal cancer, a liver cancer, a kidney cancer, a bladder cancer, a pancreatic cancer, an ovarian cancer, a testicular cancer, a prostate cancer, an endometrial cancer, a muscle cancer, a bone cancer, a neuroendocrine cancer, a connective tissue cancer, a head or neck cancer, or a skin cancer. In some embodiments, a NPM1-expressing cancer is acute myeloid leukemia (AML), acute promyeloid leukemia (APL), acute lymphoblastic leukemia (ALL), non-Hodgkin's lymphoma, and myelodysplastic syndrome (MDS). In some embodiments, a NPM1-expressing cancer is a metastatic cancer. In some embodiments, a NPM1-expressing cancer is a therapy-related cancer or a secondary malignancy. In some embodiments, a NPM1-expressing cancer is therapy-related AML (t-AML) or a secondary malignancy of non-Hodgkin's lymphoma.

In some embodiments, the biological sample collected from the subject comprises a tissue biopsy, a blood sample, a serum sample, a plasma sample, a saliva sample, a sputum sample, a urine sample, a fecal sample, a lymphatic fluid sample, a synovial fluid sample, a cerebrospinal fluid sample, or an interstitial fluid sample. In some embodiments, a tissue biopsy is a tumor biopsy. In some embodiments, the biological sample collected from the subject is a blood sample, a serum sample, or a plasma sample.

In some embodiments, the analysis conducted is luminescent analysis or fluorescent analysis. The method of analysis may be selected based on the type of imaging agent conjugated to an anti-NPM1 antibody described herein. For example, in some embodiments, the anti-NPM1 antibody is conjugated to a luminescent or fluorescent imaging agent. In some embodiments, the analysis is performed via flow cytometry. Various techniques for assessing binding between a labeled antibody (e.g., an anti-NPM1 antibody conjugated to a luminescent or fluorescent imaging agent) and cells in a sample (e.g., NPM1-expressing cancer cells) using flow cytometry are well known in the art (see, e.g., McKinnon, K. M., “Flow Cytometry: An Overview.” Curr Protoc Immunol. 2018; 120:5.1.1-5.1.11, the contents of which are incorporated by reference herein).

Alternative approaches for analyzing binding by an antibody or antibody conjugate and cells in a sample are also well known in the art and are further contemplated herein. For example, binding between an antibody or a conjugate described herein and NPM1-expressing cancer cells in the sample may alternately be analyzed by enzyme-linked immunosorbent assay (ELISA). In some embodiments, binding between an antibody or a conjugate described herein and NPM1-expressing cancer cells in the sample is analyzed by cell-based ELISA using techniques that are well known in the art (see, e.g., Molnár E., “Cell-Based Enzyme-Linked Immunosorbent Assay (Cell-ELISA) Analysis of Native and Recombinant Glutamate Receptors.” Methods Mol Biol. 2019; 1941:47-54, the contents of which are incorporated by reference herein). In some embodiments, the total level of NPM1 (WT or mutant NPM1) expressed by cells in the sample (e.g., cancer cells) and/or the level of NPM1 (WT or mutant NPM1) localized on the surface of cells in the sample (e.g., cancer cells) may be further analyzed using techniques that are well known in the art, such as, for example, fractionation of cellular membranes followed by immunoblotting for NPM1, e.g., by using one or more anti-NPM1 antibodies described herein.

In some embodiments, a method of evaluating the presence of (detecting) an NPM1-expressing cancer in a subject further comprises administering an effective amount of an anti-NPM1 antibody, a conjugate comprising an anti-NPM1 antibody and an agent (e.g., a drug, a radioisotope) described herein, or a composition thereof (e.g., a pharmaceutical composition) to the subject, for example, if a NPM1-expressing cancer is determined to be present in the subject. The effective amount of an anti-NPM1 antibody, a conjugate comprising an anti-NPM1 antibody and an agent (e.g., a drug, a radioisotope), or a composition thereof (e.g., a pharmaceutical composition) to be administered to the subject may depend on a variety of factors that are within the knowledge and expertise of the health practitioner to determine, including the amount and location of the NPM1-expressing cancer that is determined to be present in the subject.

Those of skill in the art will recognize that the methods of treatment and detection described herein may also be suitable for the treatment or detection of other diseases or disorders associated with a cell expressing cell surface NPM1, for example, noncancerous diseases or disorders associated with cell surface expression of WT NPM1 or mutant NPM1.

### EXAMPLES

### Example 1—NPM1 is Localized on the Surface of Human Leukemia Cells

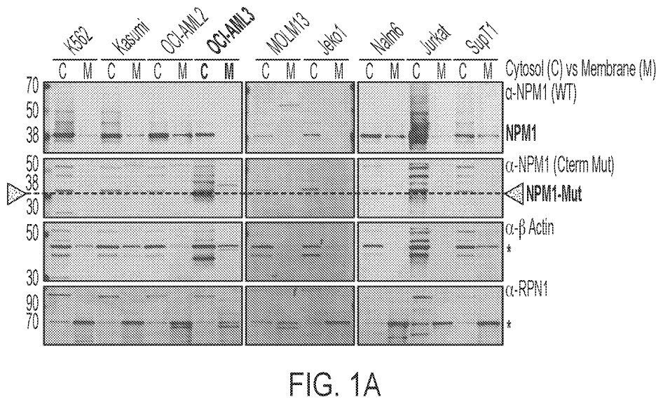

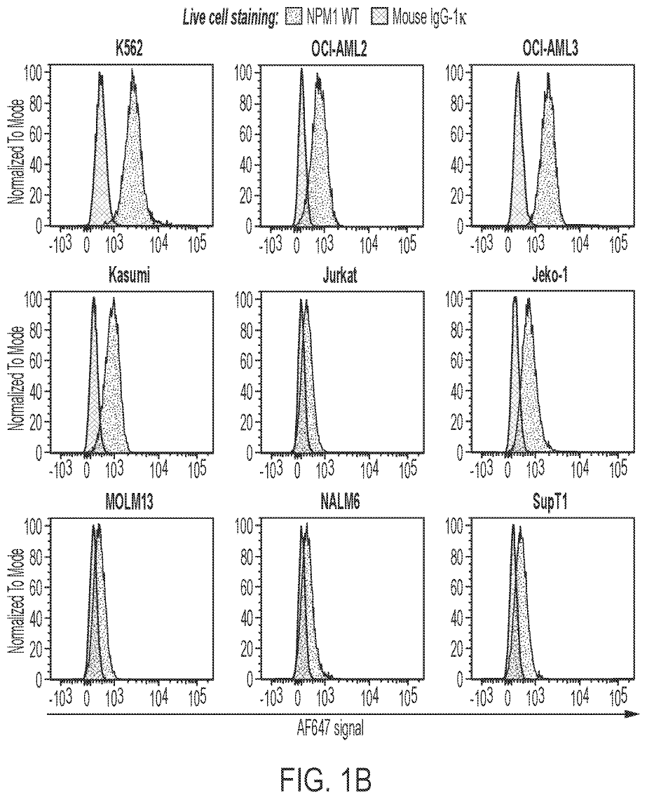

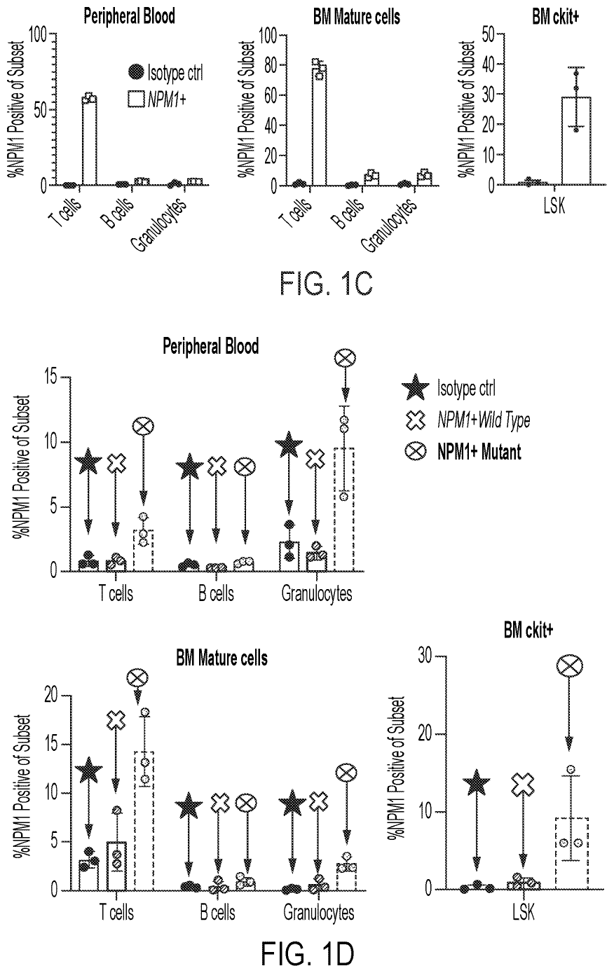

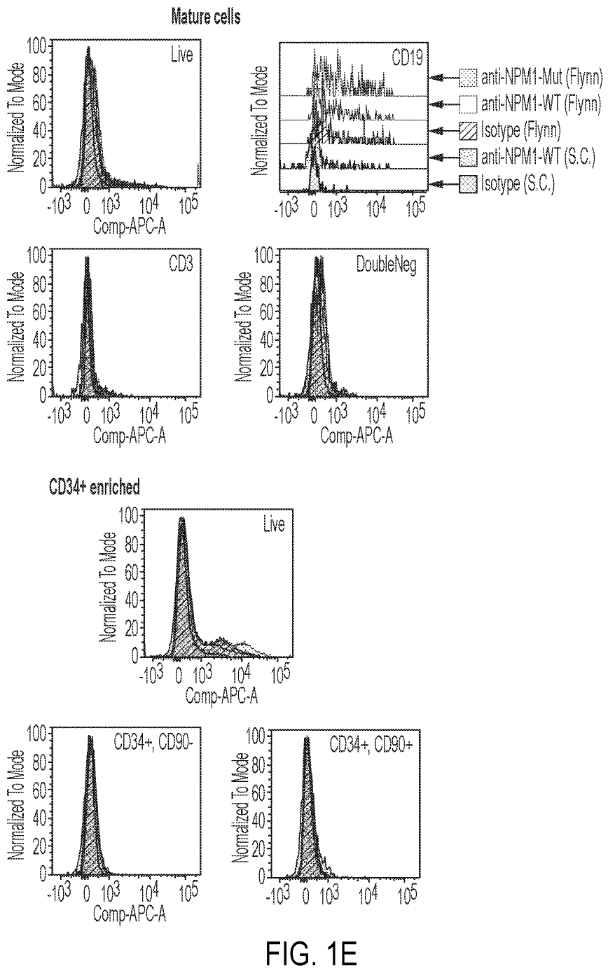

Various human leukemia cell lines were evaluated for cell surface expression of nucleophosmin 1 (NPM1). Briefly, K562, Kasumi, OCI-AML2, OCI-AML3, MOLM13, Jeko1, Nalm6, Jurkat, and SupT1 cells were cultured in vitro with RPM1 media supplemented with 10% fetal bovine serum (FBS) and 1% penicillin-streptomycin (Pen/Strep). Cellular membrane and cytosolic fractions were collected using established techniques (see, e.g., Flynn et al., Cell. 2021; 184(12):3109-3124.e22) and immunoblotted with the following commercial antibodies: NPM1-WT (Santa Cruz Biotechnology #sc-32256), NPM1-Mut (Thermo Scientific #PA1-46356), beta-actin (Santa Cruz Biotechnology #sc-47778), and RPN1 (Santa Cruz Biotechnology #sc-48367). Although the majority of NPM1 was localized in the cytosol of each cell type, significant quantities of wild-type (WT) and mutant (Mut) NPM1 was identified on the surface of all cell types (FIG. 1A). Further fluorescence activated cell sorting (FACS) analysis of live cells confirmed varying levels of NPM1 on the surface of each cell type that was assayed.

These results suggest that an antibody that is specific for WT and/or mutant NPM1 could be used to carry cytotoxic payloads to NPM1-expressing cancers in patients, such as, for example, in patients with acute myeloid leukemia (AML), non-Hodgkin's lymphoma, and myelodysplastic syndrome (MDS). However, such a treatment strategy depends on the levels of NPM1 expressed on the surface of cancer cells being greater that that expressed on the surface of noncancer cells, especially on the surface of cells from which the cancer was initially derived. Therefore, the level of NPM1 on the surface of peripheral blood (PB) and bone marrow (BM) cells was assessed. In most murine PB and BM cells tested, NPM1 cell surface expression was relatively low, but was increased in cells expressing mutant NPM1 (FIGS. 1C and 1D). These results were confirmed by FACS analysis of human BM cells, in which an antibody specific for WT NPM1 did not effectively bind human BM cells (FIG. 1E). These results strongly suggest that an antibody specific for NPM1 could be used to effectively treat NPM1-expressing cancers.

### Example 2—Development of Multiple Antibodies Specific for NPM1

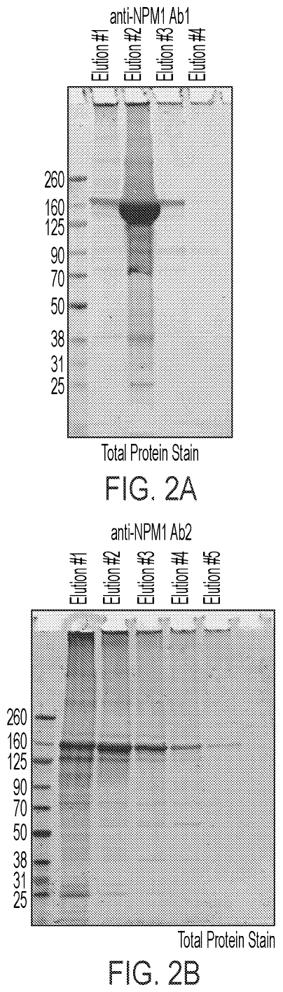

To further assess the possibility that antibodies specific for NPM1 could be used to treat NPM1-expressing cancers, novel antibodies specific for NPM1 were generated. Distinct antibodies were generated having specificity for NPM1 (“Ab1” and “Ab2”; FIGS. 2A and 2B).

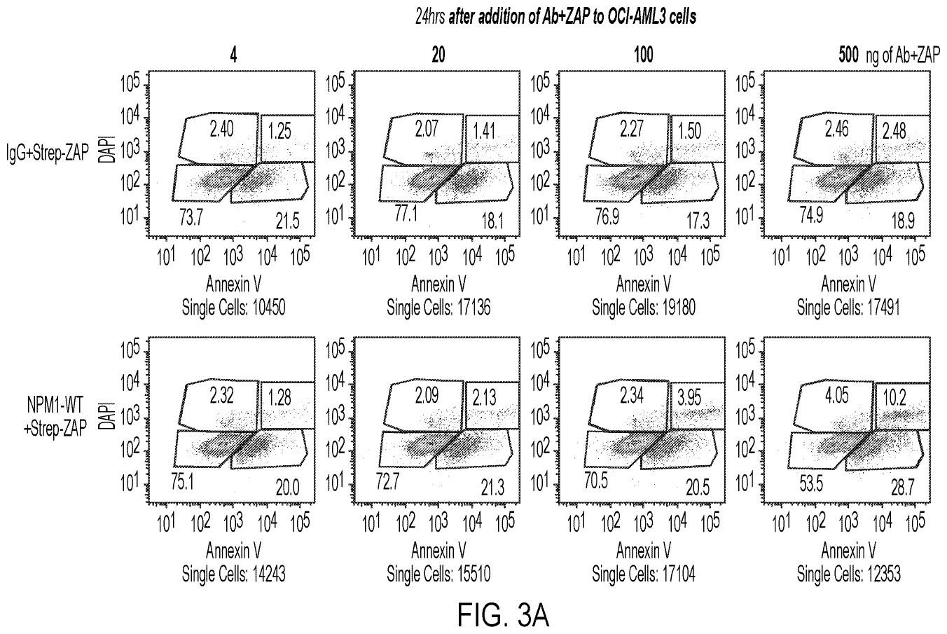

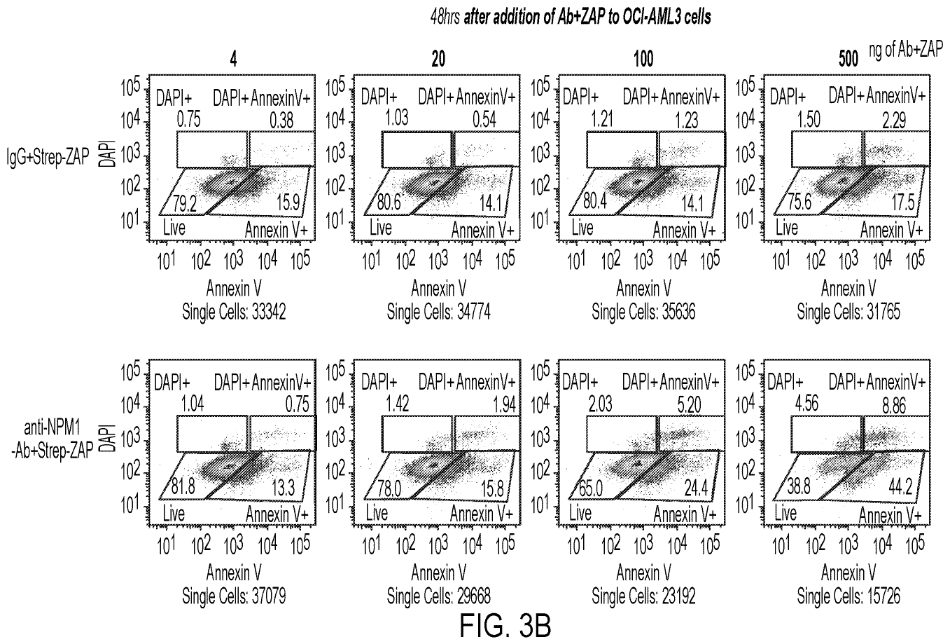

In order to assess whether or not the isolated antibodies could be useful for the treatment of cancers, the Ab1 anti-NPM1 antibody was arranged in an antibody-drug conjugate and used to treat OCI-AML3 cells in vitro. OCI-AML3 cells are a model for human acute myeloid leukemia (AML). Briefly, approximately 150,000 cells were cultured in RPMI media with 10% FBS and 1% Pen/Strep. Streptavidin-Saporin (Strep-ZAP) was bound to biotinylated antibodies at 2.6 μg Strep-ZAP to 1 μg IgG for 30 minutes on ice. Strep-ZAP was bound to either biotinylated Ab1 anti-NPM1 antibody (FIG. 2A) or to biotinylated-anti-mouse isotype control (Strep-ZAP-IgG) (Thermo Scientific #31800). After binding, Strep-ZAP-IgG complexes were added to cells and cells were left to grow at 37° C. for either 24 hours or 48 hours. After each time point, cells were harvested, washed in PBS, stained with AnnexinV to detect apoptosis, and stained with DAPI to detect if cells were alive. After staining, cells were washed and analyzed by FACS. After incubation, killing of OCI-AML3 cells was observed in a dose-dependent manner, with fewer live cells observed after 48 hours at the highest dose (FIGS. 3A and 3B). Critically, less apoptosis and cell death were observed with the same amount of Strep-ZAP lacking cell surface targeting (Strep-ZAP-IgG samples). These results confirm the utility of ADCs based upon anti-NPM1 antibodies for targeting cytotoxic drugs to cancer cells that express cell surface NPM1.

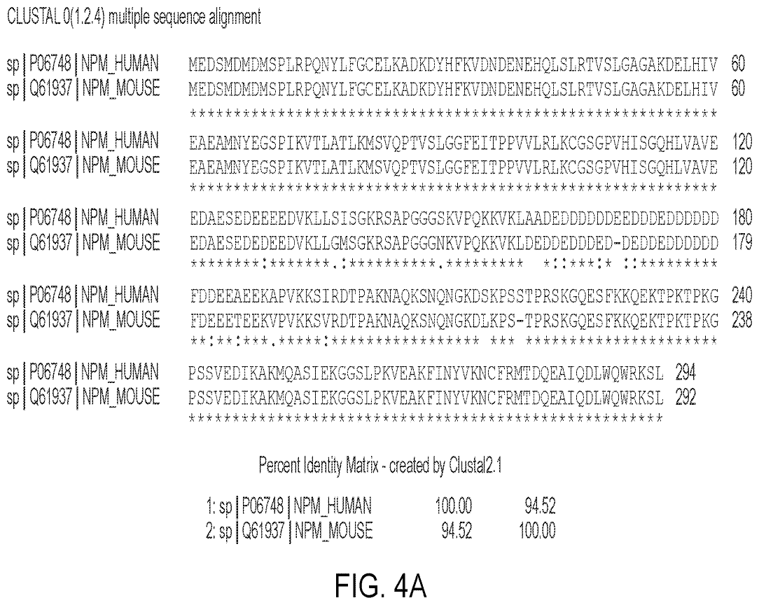

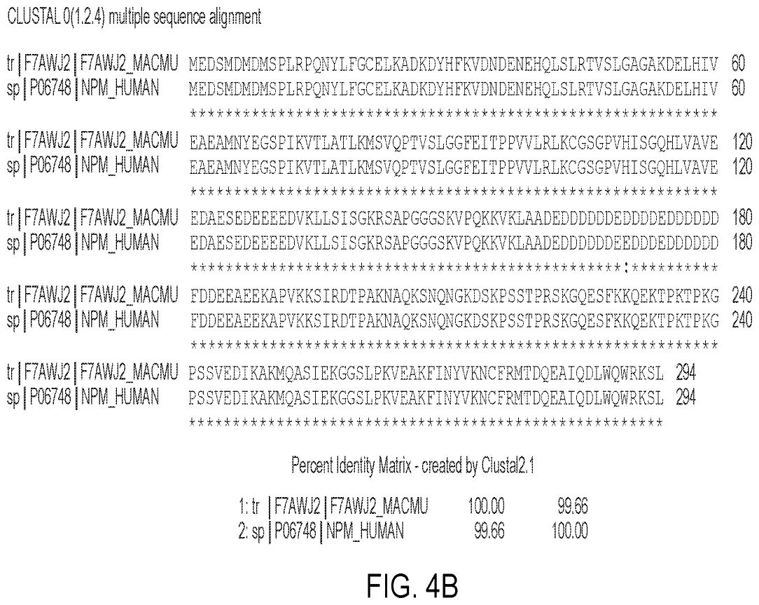

Although these studies were conducted with a human cell line, it should be noted that NPM1 antibodies could be used to target endogenous NPM1 on the surface of non-human cell types, due to the high degree of conservation of NPM1. Notably, the amino acid sequence of WT NPM1 is approximately 95% identical between humans and mice (FIG. 4A), and greater than 99% identical between humans and other primates (FIG. 4B). The high degree of conservation also suggests that results obtained by testing the effect of anti-NPM1 antibodies or ADCs in, for example, murine cells or in mice, would be expected to extend to humans.

### Example 3—Antibodies Detect WT and Mutant NPM1 on the Surface of Human and Murine Models

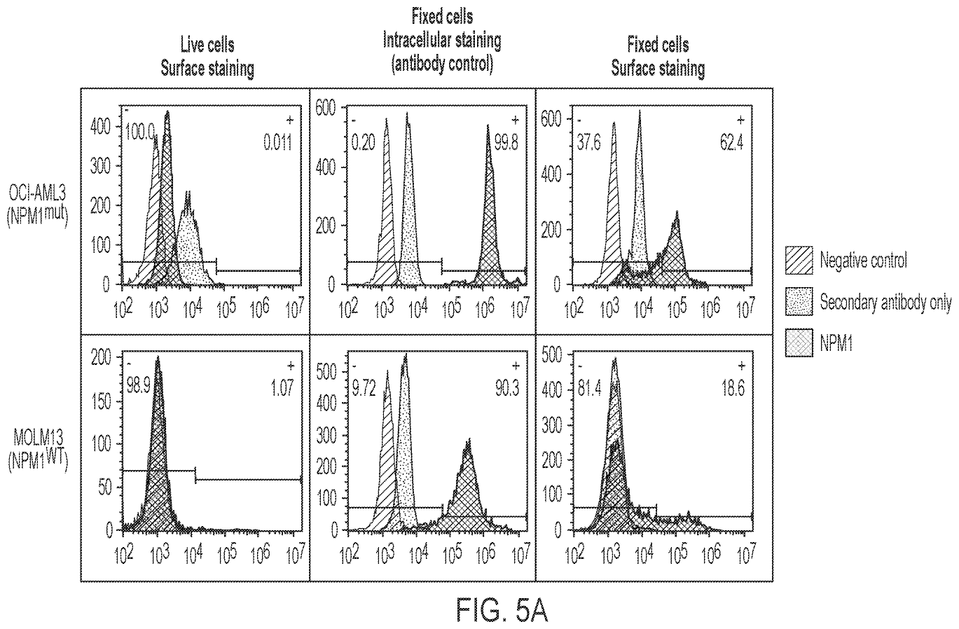

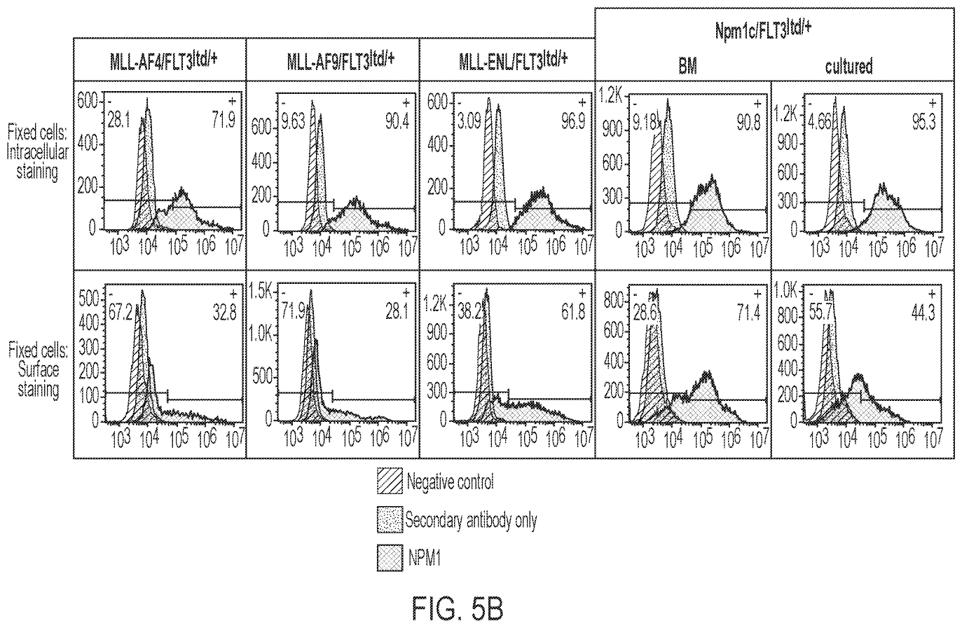

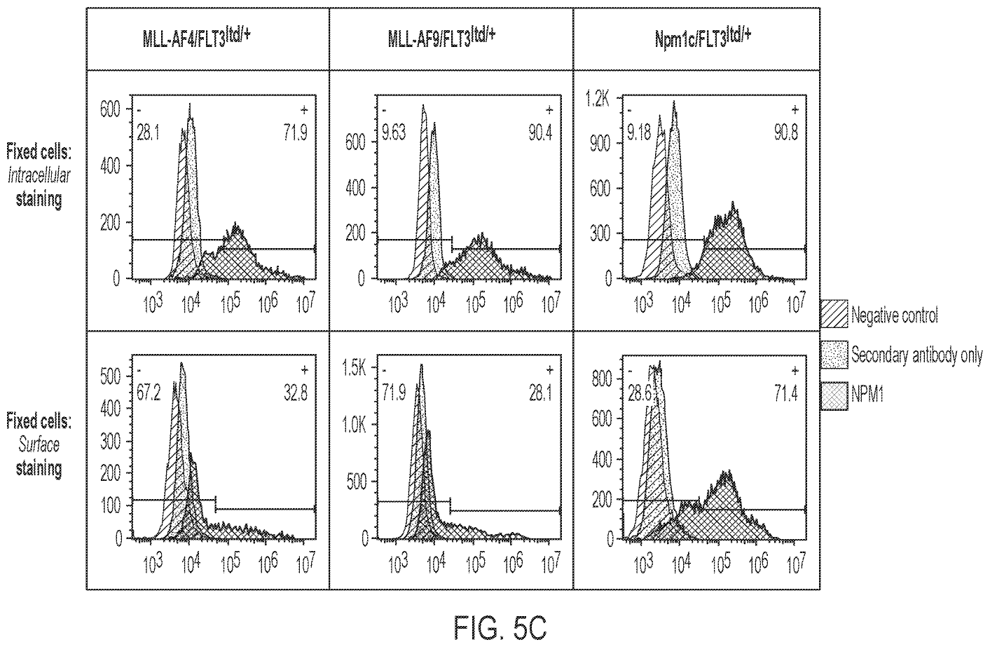

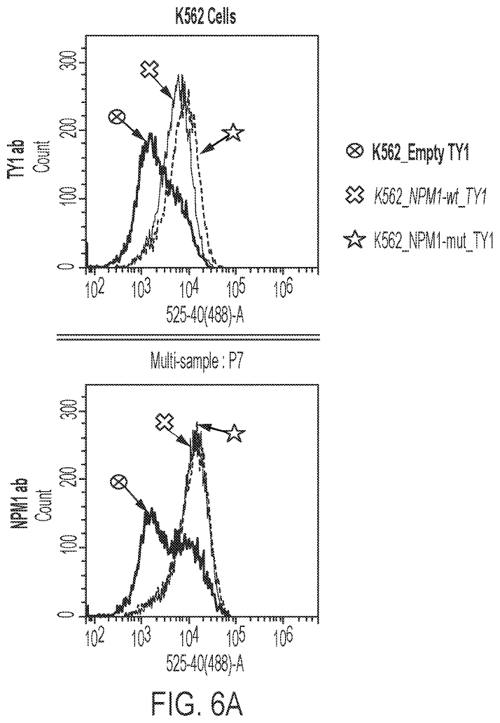

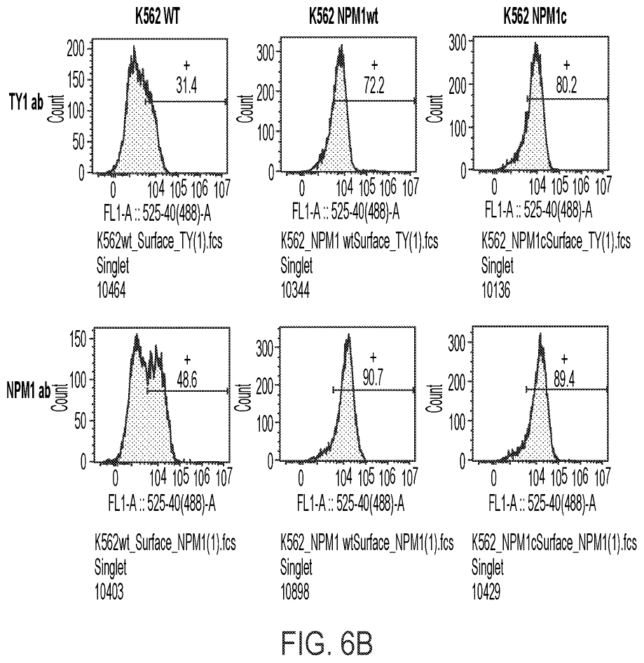

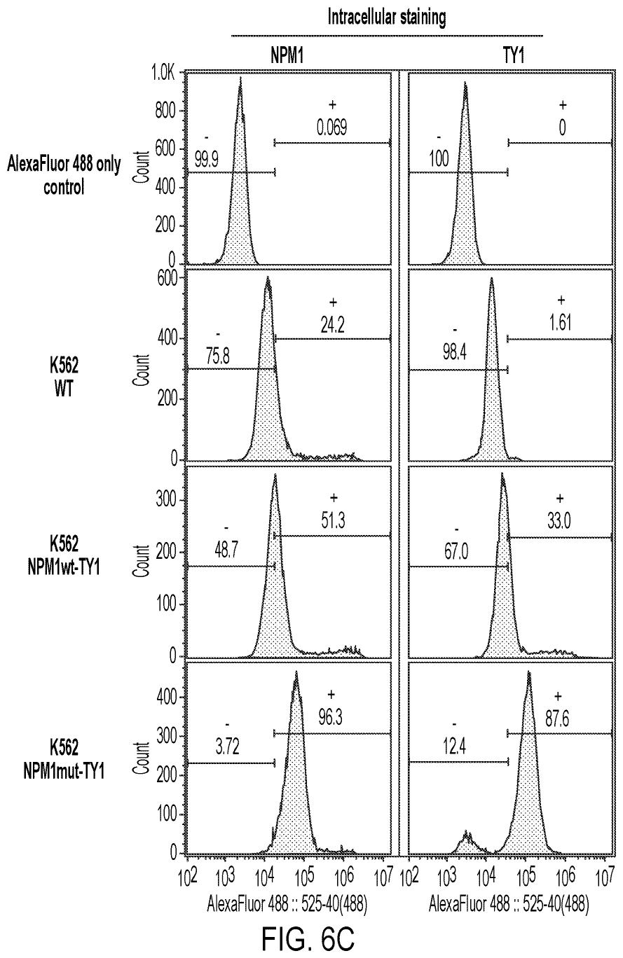

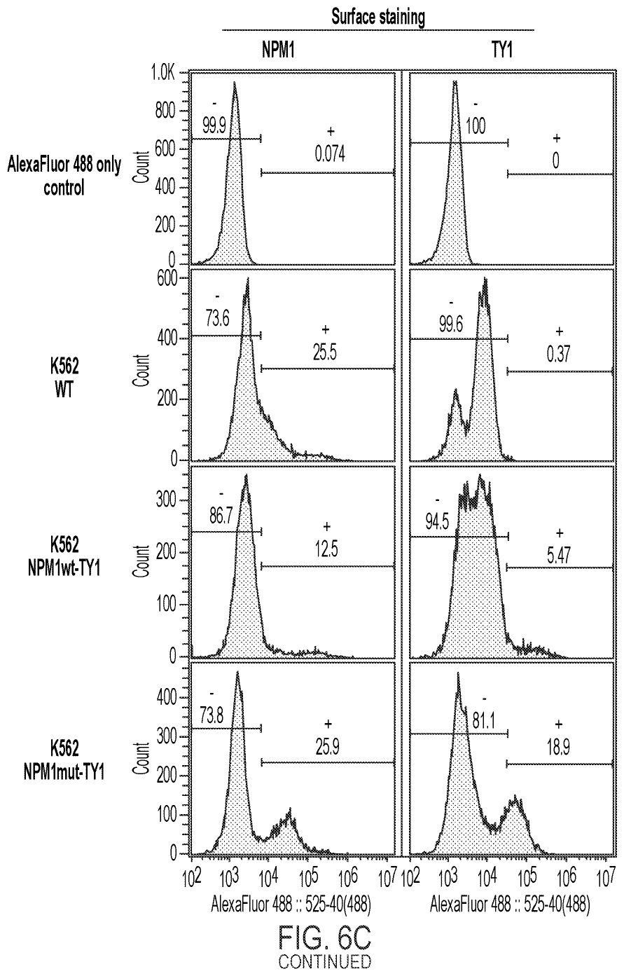

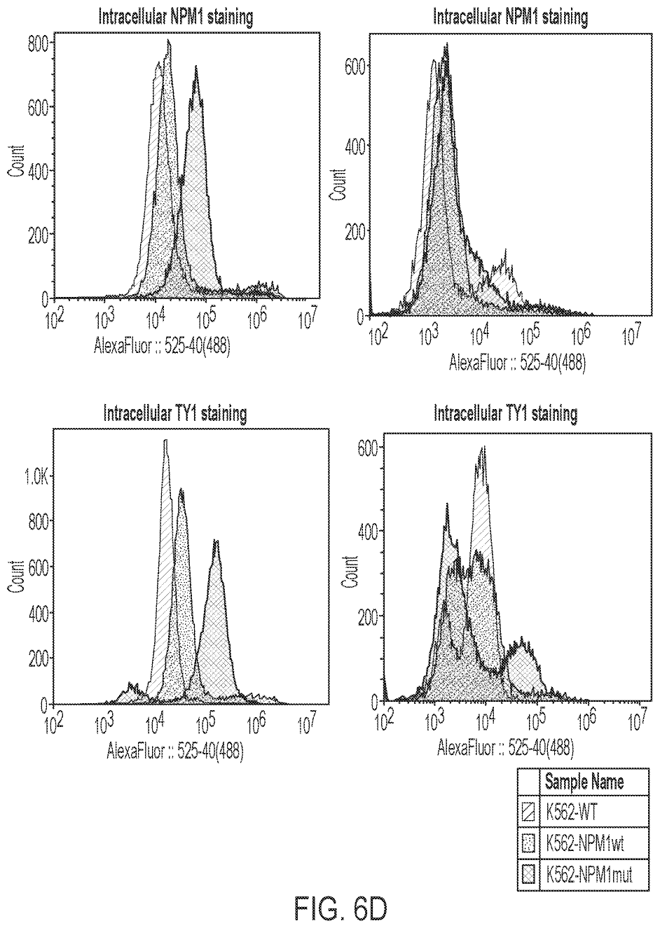

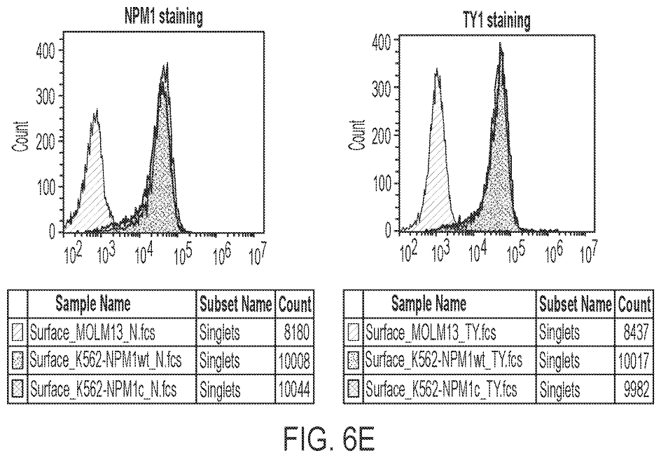

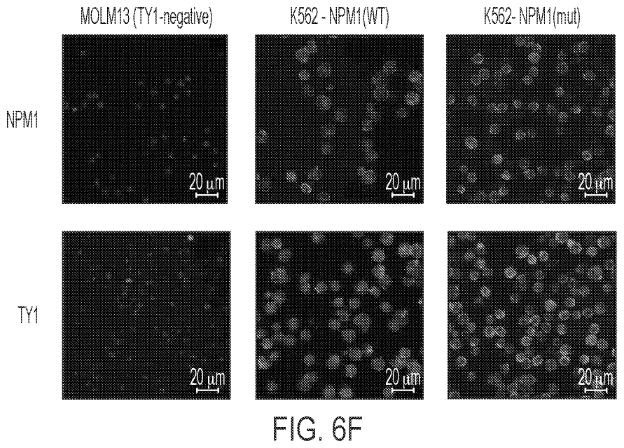

To further establish that NPM1 antibodies could be used to target NPM1 on cancer cells, the level of NPM1 on the surface of various human and murine cancer cells was explored using FACS analysis. In some instances, live cells were assayed to observe cell surface localized NPM1. In other instances, fixed cells were assayed to observe intracellular and cell surface levels of NPM1. Significant levels of NPM1 were observed on the surface of live OCI-AML3 cells, however NPM1 was not robustly observed in significant quantities on MOLM13 cells (FIG. 5A). Similar to OCI-AML3 cells, significant levels of NPM1 were observed on the surface of various primary murine AML cell lines (FIGS. 5B and 5C). To further explore cell surface NPM1 in non-AML cell lines, human K562 cells, which are a model for myelogenous leukemia, were also assessed. In these experiments, exogenous NPM1 was expressed with a TY1 tag, where the exogenous NPM1 was either WT NPM1 or mutant NPM1 (NPM1c). Using live cell and fixed cell flow cytometry, TY1 signal was found to be present on the surface of cells expressing either WT-NPM1-TY1 or NPM1c-TY1, confirming that the NPM1c protein can also be presented on the cell surface (FIGS. 6A-6E). An orthogonal immunofluorescence detection strategy confirms the surface distribution of the TY1-tagged NPM1 molecules (FIG. 6F).

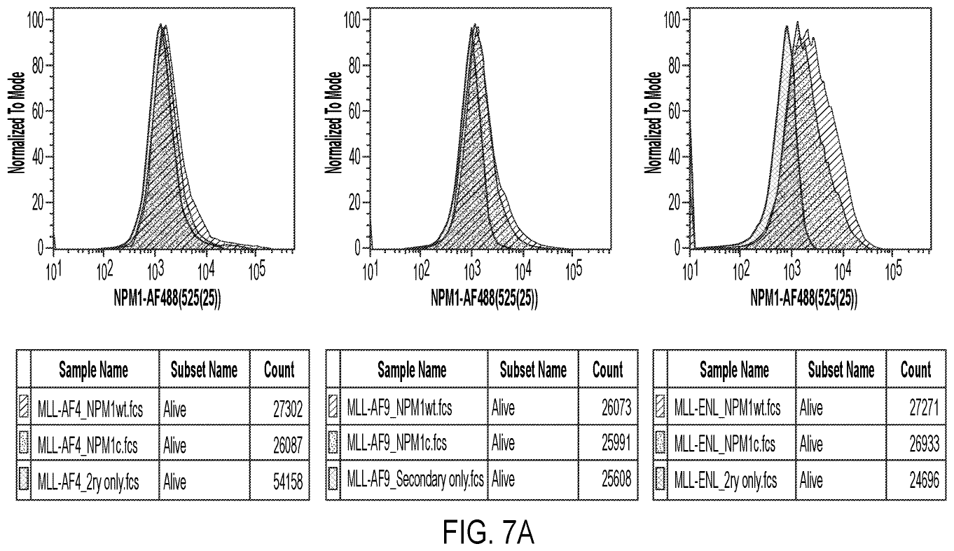

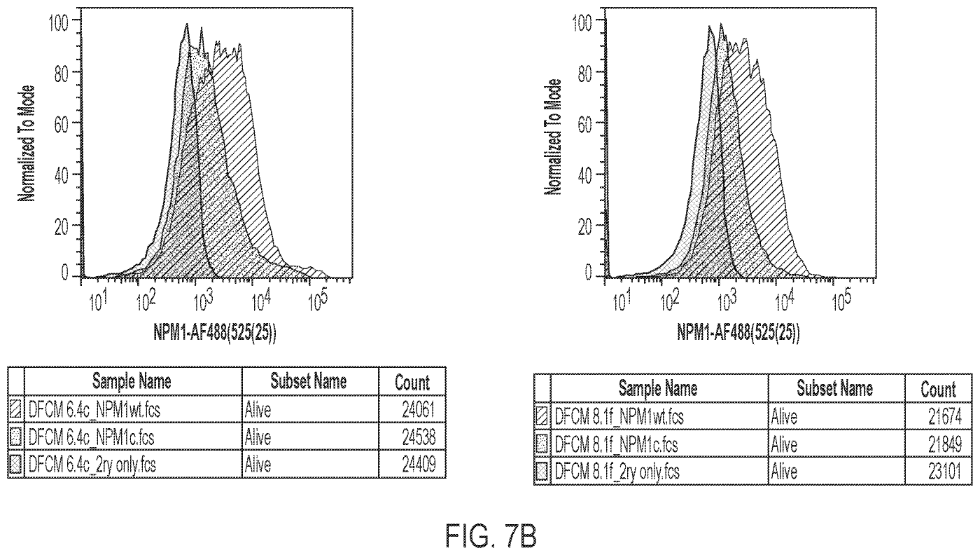

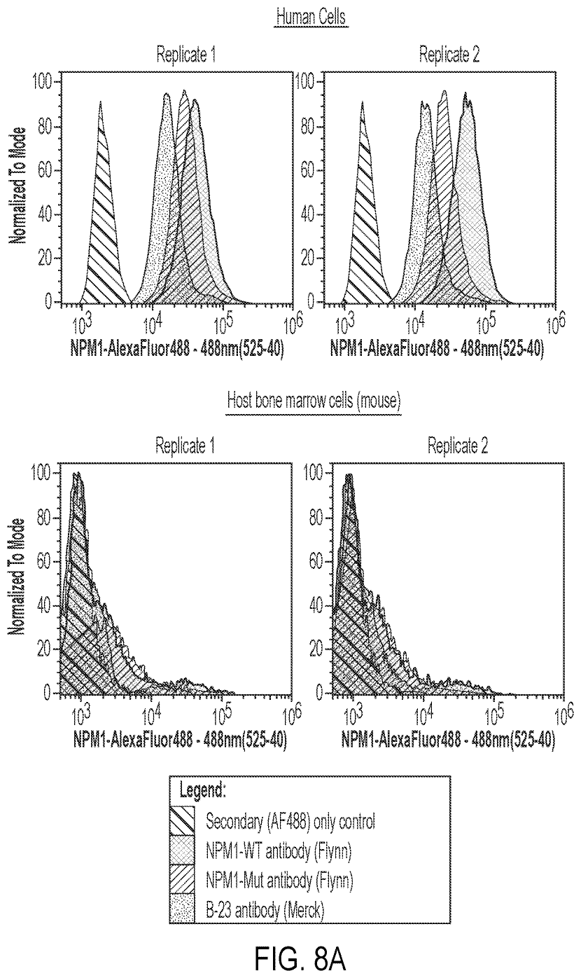

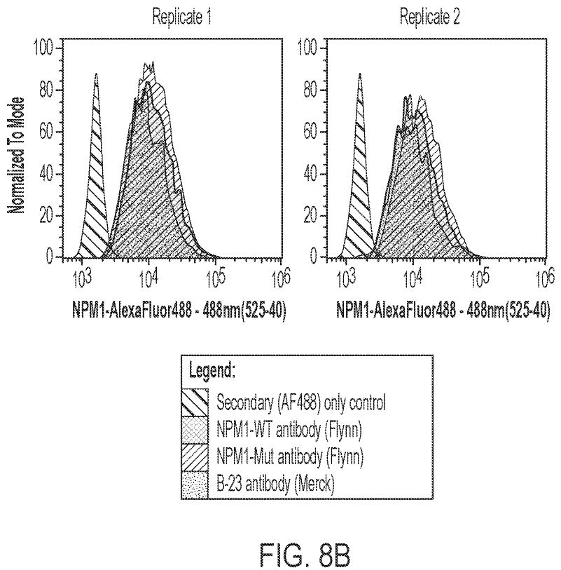

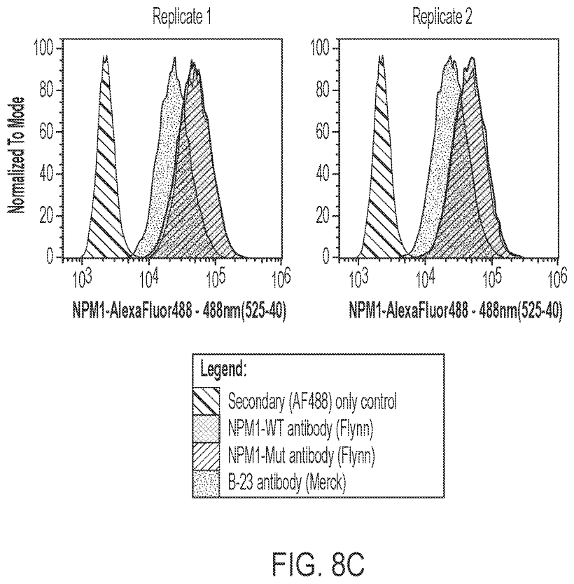

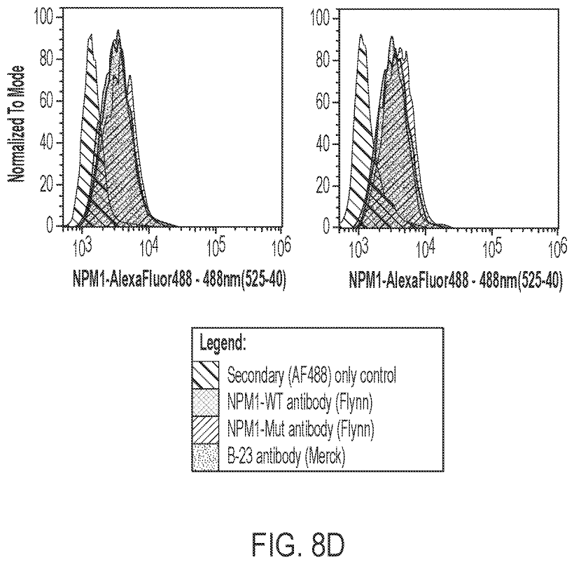

Given that a variety of murine and human leukemia cell lines express cell surface WT and/or mutant NPM1, it was subsequently assessed whether the isolated antibodies could also bind to the surface of these cells. Isolated Ab1 anti-NPM1 (FIG. 2A) and Ab2 anti-NPM1 (FIG. 2B) antibodies were each capable of binding to surface localized NPM1 on primary murine MLL-rearranged AML cells (FIG. 7A) and primary murine NPM1c AML cells (FIG. 7B), as determined by FACS analysis. Furthermore, the antibodies bound to NPM1 on human patient derived xenograft (PDX) cells transplanted into a mouse model, without binding to host murine bone marrow cells (FIGS. 8A-8D). These results, in combination with those above, provide additional evidence that ADCs comprising either of these anti-NPM1 antibodies could be used to selectively direct chemotherapeutic agents to cancer cells, with low risk of toxicity against noncancerous tissue. These antibodies could further be used in other applications, including, for example, diagnostic agents by instead conjugating the antibodies with an imaging agent, which can in turn be used to locate and measure the relative abundance of cancer cells in a patient.

### Example 5: NPM1 is a Cell Surface Protein

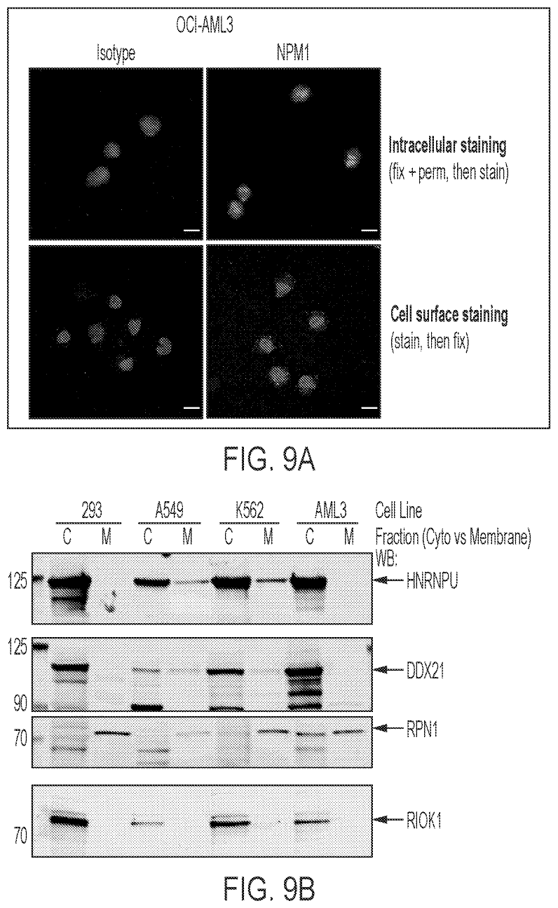

To assess the subcellular localization of nucleophosmin 1 (NPM1) confocal imaging was performed on OCI-AML3 (human cell line). Both intracellular and cell surface staining was performed. Intracellular staining was achieved by fixing and permeabilizing the cells prior to staining. Comparatively, cell surface staining was performed by staining the cells prior to fixation. The results of the staining show that NPM1 is found both inside the cells and on the cell surface. Inside the cells, NPM1 is nucleolar and on the cell surface, NPM1 forms clusters (FIG. 9A). To investigate whether biochemical fractionation was specific, other RNA Binding Proteins (RBPs) were investigated, including Heterogeneous nuclear ribonucleoprotein U (HNRNPU), Nucleolar RNA helicase 2 (DDX21), Dolichyl-diphosphooligosaccharide protein glycosyltransferase subunit 1 (RPN1), and RIO Kinase 1 (RIOK1). The RBPs were measured in the cytosolic and membrane compartments of various cell lines, including 293, A549, K562, and AML3. Results from the biochemical fractionation and western blot (WB) show that some other RBPs, but not all highly abundant cytosolic proteins are found int the membrane fractions of various cells (FIG. 9B).

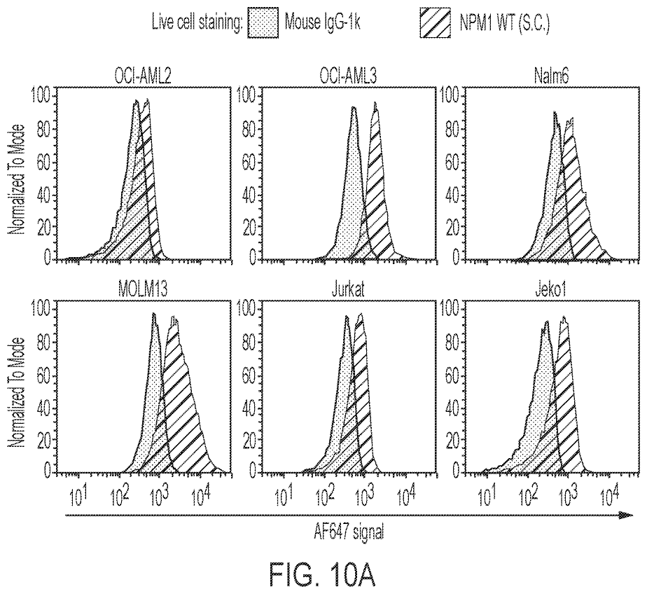

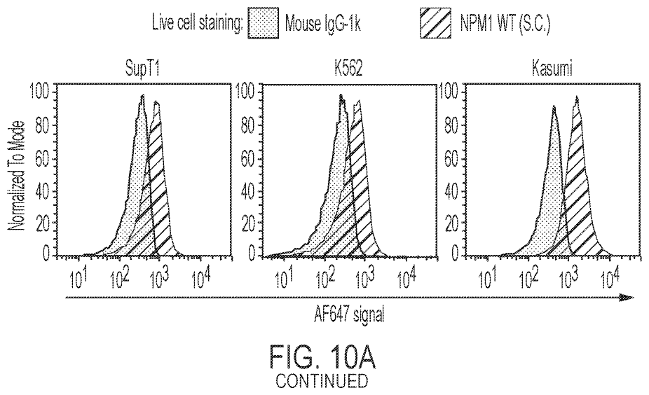

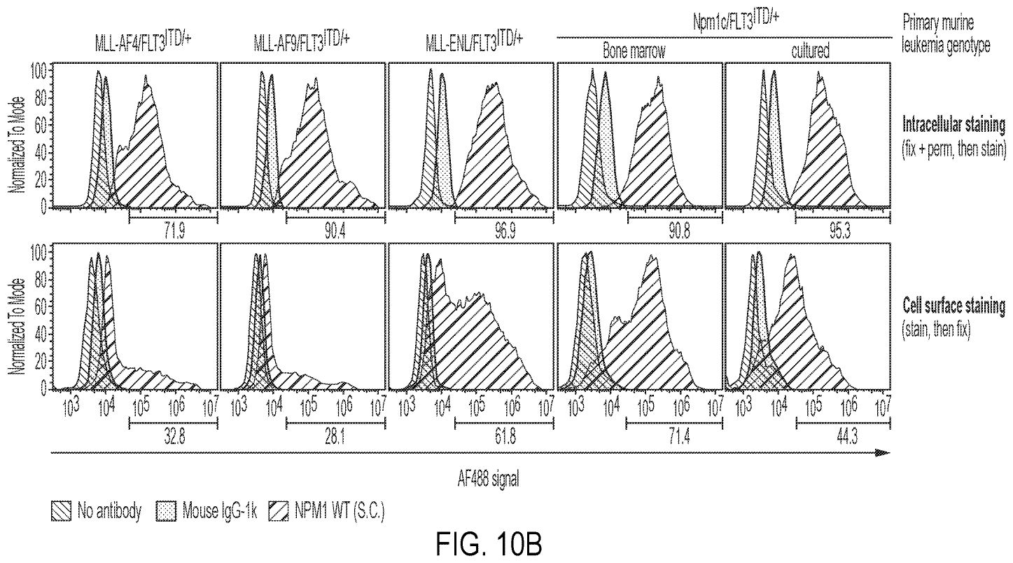

In addition to assessing cell surface expression of NPM1 on human cells, primary murine acute myeloid leukemia (AML) cells were also investigated using live cell flow cytometry. Like the human OCI-AML3 cell line, cell surface expression of NPM1 was detected on primary murine AML cells (FIGS. 10A-10B).

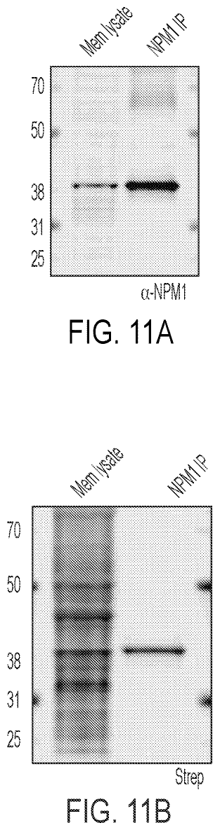

The cell surface localization of NPM1 was also tested using western blot. anti-NPM1 captures full length NPM1 from cellular membrane fractions (FIG. 11A). Cell surface NHS-biotinylation (only surface proteins) followed by anti-NPM1 IP from membrane fractions selectively isolates a biotinylated band at the molecular weight of NPM1 (FIG. 11B).

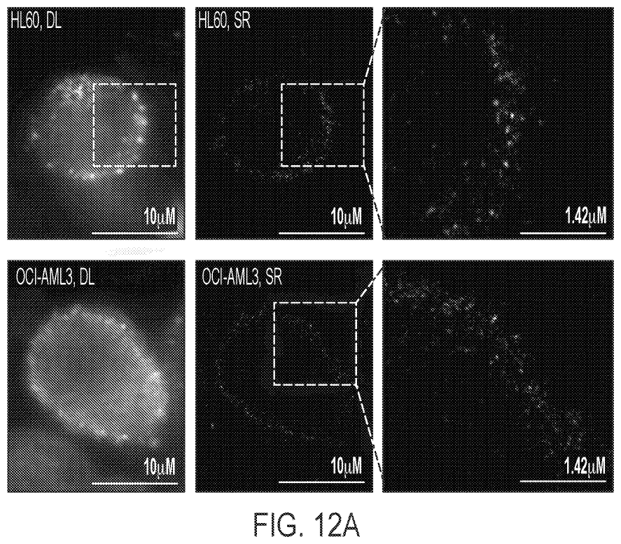

Further, to assess the ability of NPM1 to form nanoclusters on the cell surface super resolution microscopy was performed on HL-60 (Human Leukemia Cell Line) and OCI-AML3 cells. Super resolution microscopy defines 120-150 nanometer (nm) clusters. These NPM1 clusters were present on the cell surface of both HL-60 and OCI-AML3 cell lines. The clustered and nonrandom patterns of the NPM1 marker suggest a highly regulated cell biology process (FIGS. 12A-12B). Super resolution microscopy performed on PANC1 cell line (pancreatic carcinoma) also demonstrated NPM1 forms nanoclusters on the surface of cancer cells (FIG. 12B).

The results of the intracellular and cell surface staining, live cell flow cytometry and western blot demonstrate that NPM1 is a cell surface protein in both human and murine cell lines. Additionally, the super resolution microscopy demonstrated that on the cell surface NPM1 forms regular nanoclusters on various human cancer cell lines, including, acute myeloid leukemia and pancreatic carcinoma.

### Example 6: Ab2.2 Antibody Targets NPM1

An antibody having the Heavy chain (Ab2.2) (SEQ ID NO: 44) and Light chain (Ab2.2) (SEQ ID NO: 45), hereinafter “Ab2.2”, was generated and used to target NPM1 on the surface of human and non-human cell types.

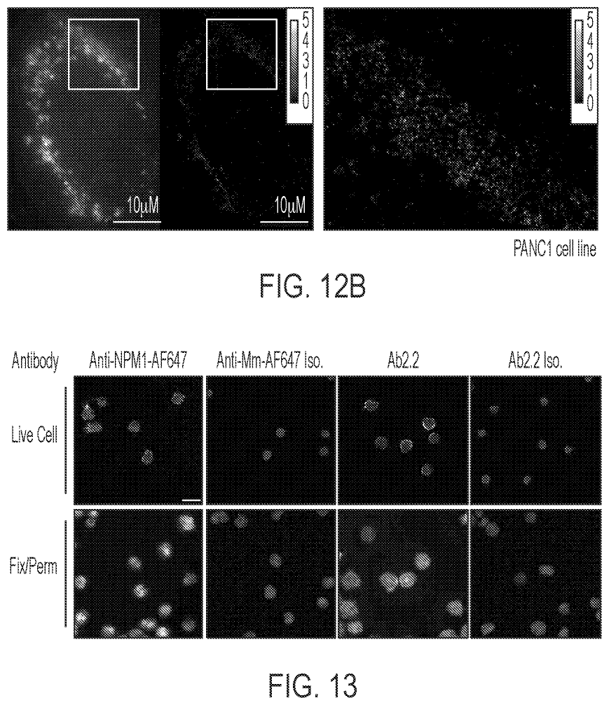

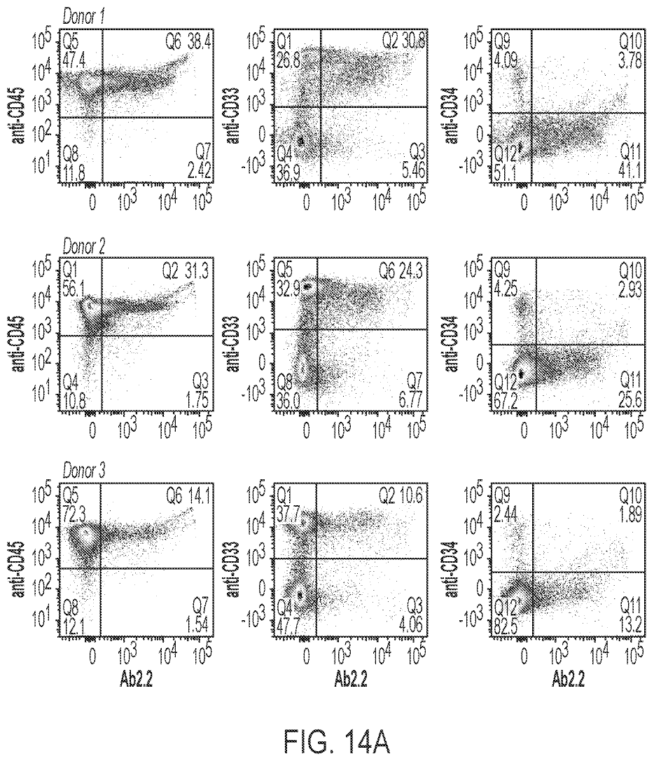

The ability of Ab2.2 to bind to NPM1 on the cell surface of cancer cells (OCI-AML3 cells) was tested using both live cell microscopy and microscopy of fixed and permeabilized cells. Ab2.2 was compared to commercially available NPM1 antibodies. The commercially available NPM1 antibody used was Santa Cruz (SC) Anti-NPM1-AF647. The live cell microscopy shows that both commercially available NPM1 antibodies and Ab2.2 stain surface puncta. Therefore, Ab2.2 performs similarly to commercially available NPM1 antibodies on the cell surface (FIG. 13). The microscopy of the fixed and permeabilized cells shows that commercially available NPM1 antibodies results in nucleolar and cytoplasmic, whereas the Ab2.2 antibody results in nucleolar and more major cytoplasmic staining. Therefore, Ab2.2 detects more cytoplasmic NPM1 in mutant OCI-AML3 cells compared to commercially available NPM1 antibodies (FIG. 13).

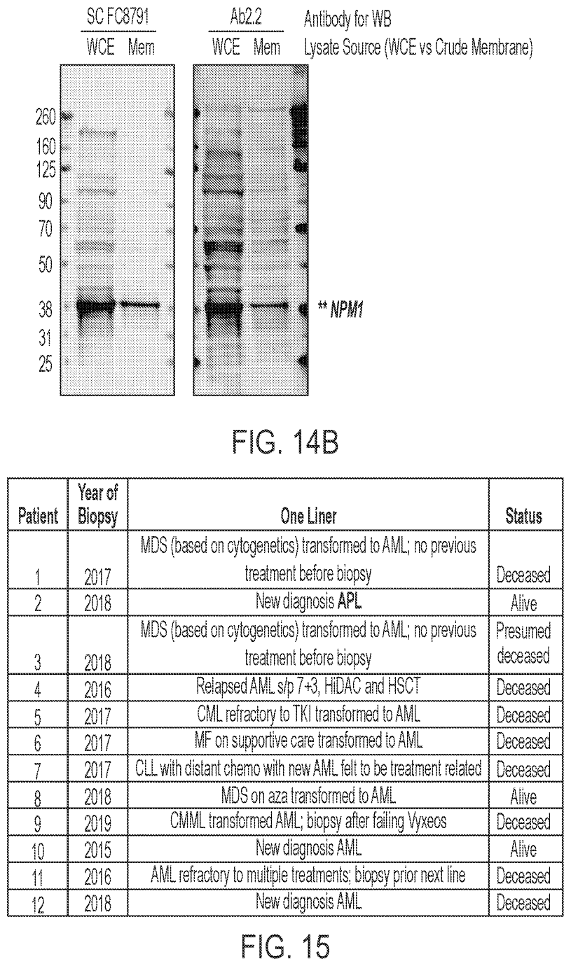

The ability of Ab2.2 to bind to NPM1 on the cell surface of healthy, non-cancerous, cells was also tested using flow cytometry. The cells measured for Ab2.2 binding were leukocytes (CD45+ cells), myeloid cells (CD33+ cells), and hematopoietic stem cells (“HSCs”; CD34+ cells). Only 3.78% of cells in Donor 1 were double positive for CD34+ and Ab2.2 (Donor 2 and Donor 3 had less than 3%). Therefore, little to no binding of Ab2.2 to healthy HSCs was observed (FIG. 14A).

The Ab2.2 antibody was further validated using western blot and compared to commercially available antibodies. The commercially available antibody was supplied from Santa Cruz (SC FC8791). Two sources of lysate were evaluated, WCE and crude membrane (Mem). Both Ab2.2 and the commercially available antibody resulted in a band around 38 kB. Therefore, commercially available and Ab2.2 show near-identical banding pattern to NPM1 (FIG. 14B).

### Example 7: Ab2.2 Targets NPM1 in Cancer Cells In Vitro

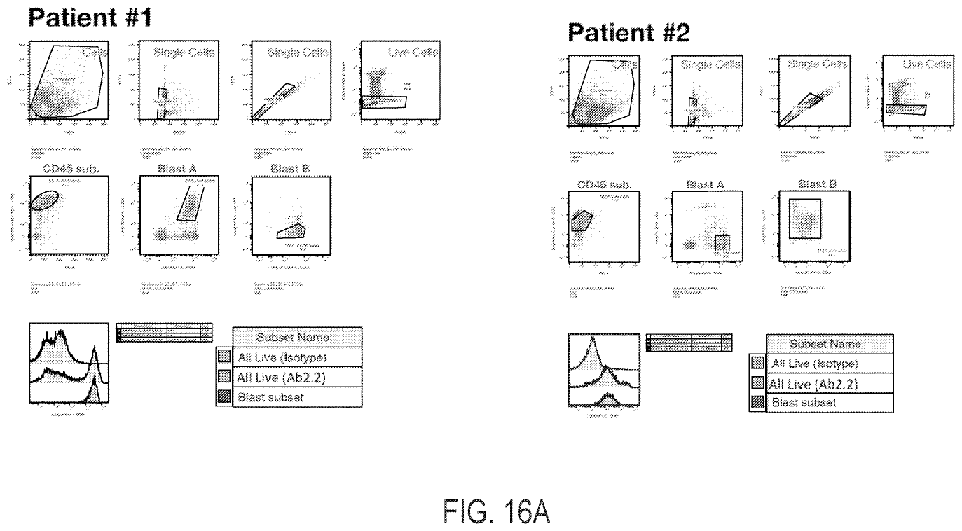

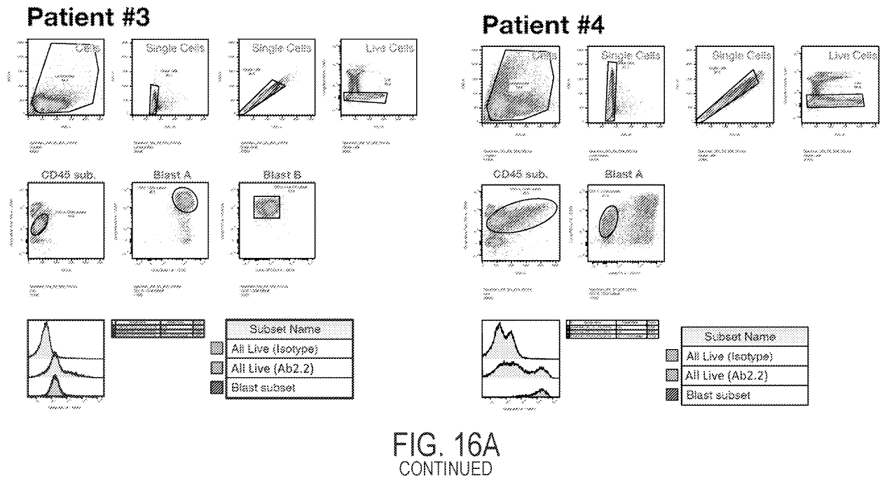

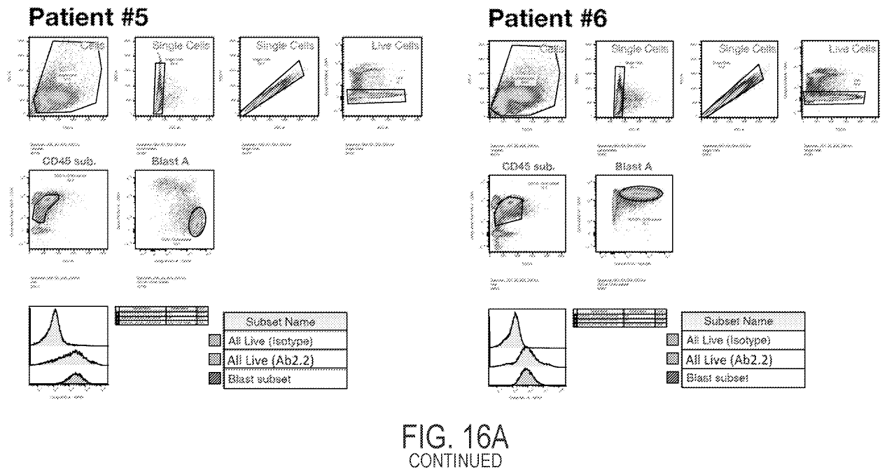

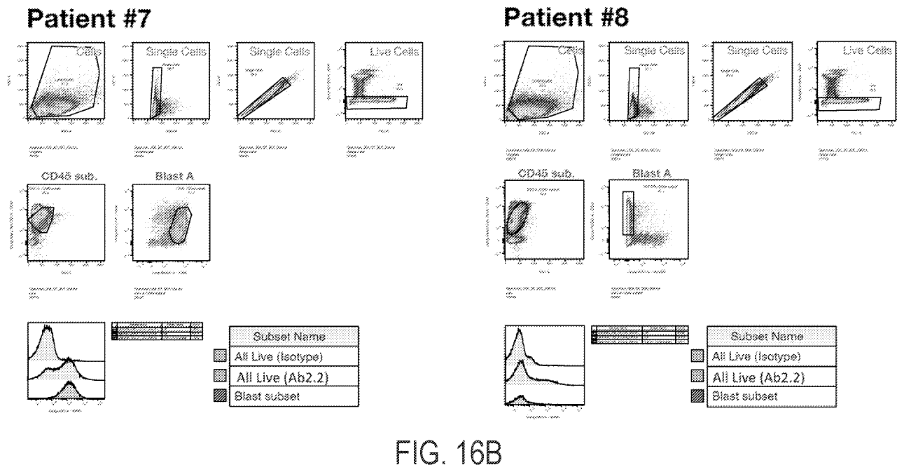

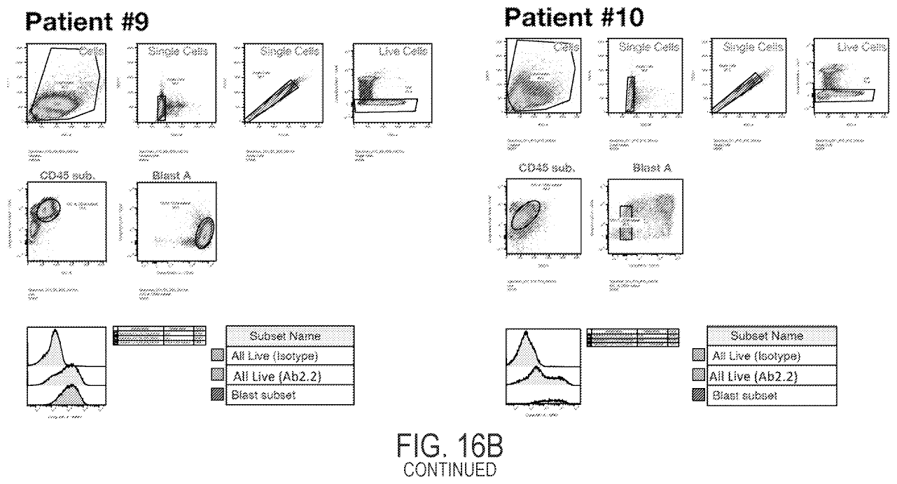

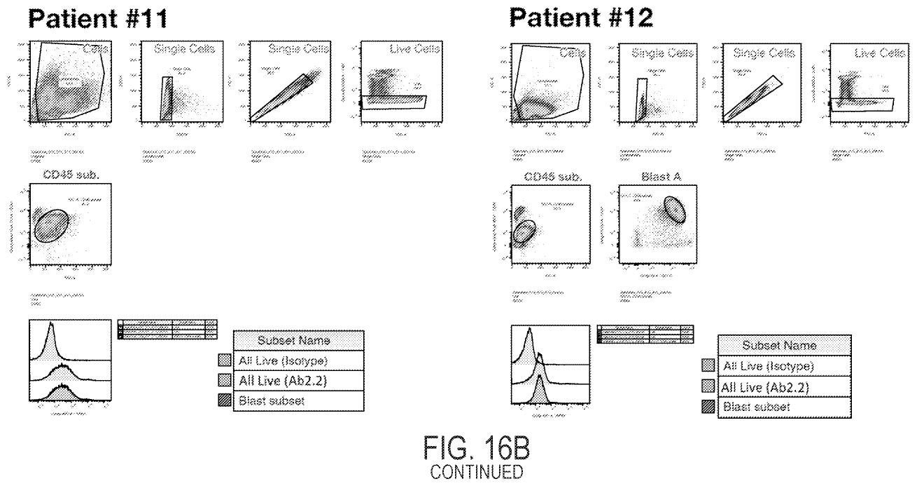

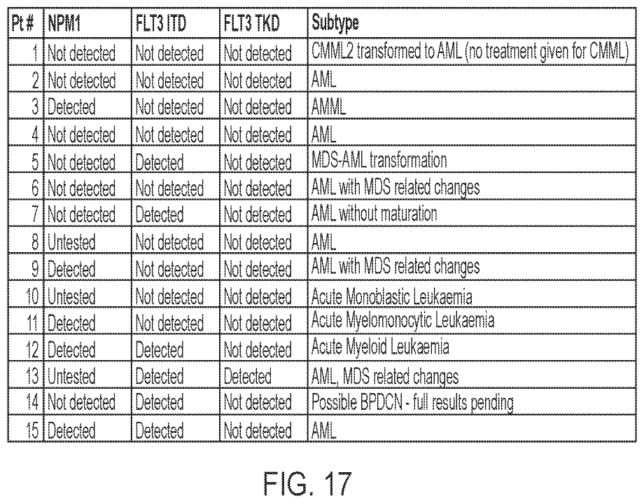

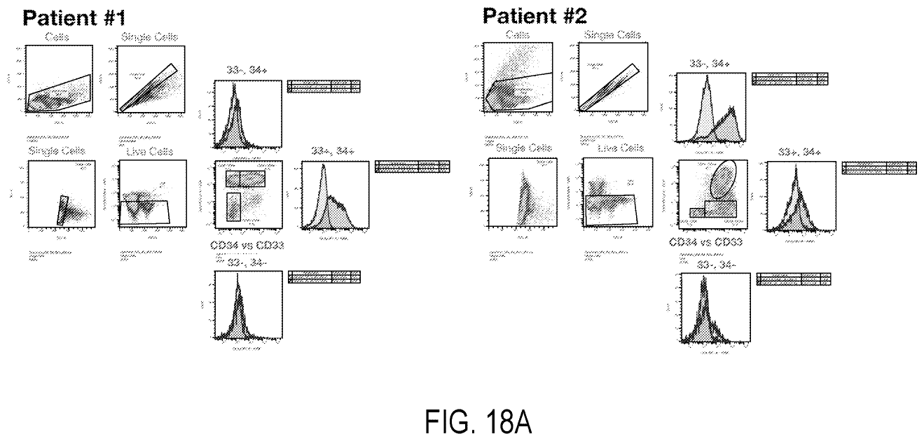

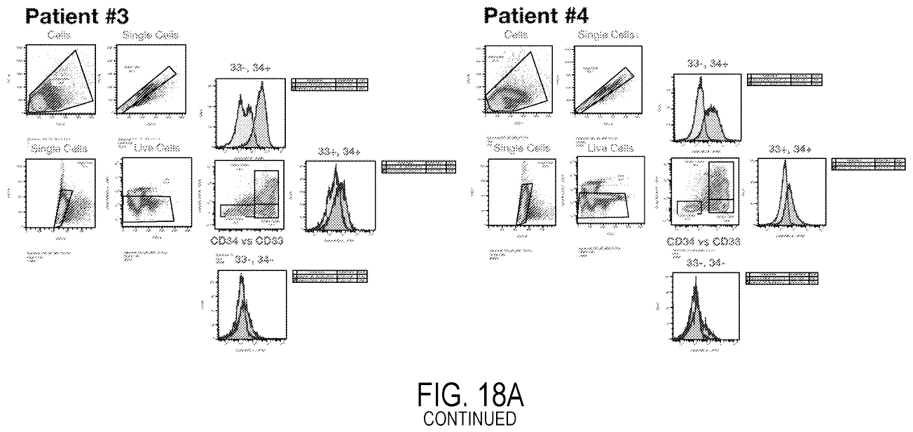

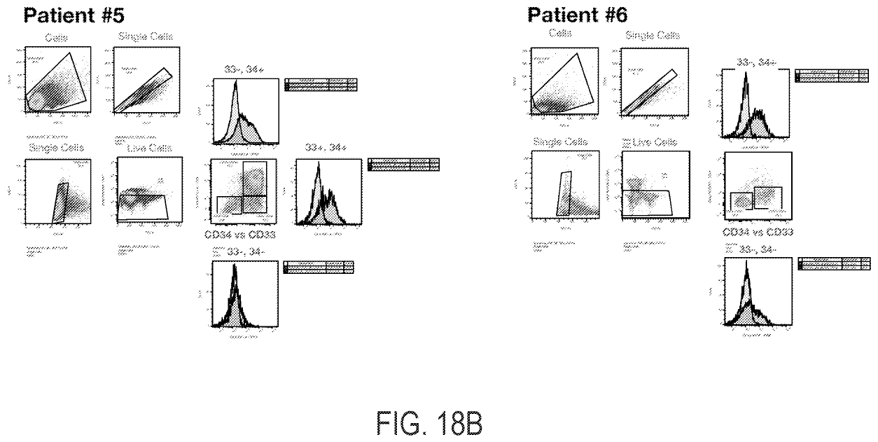

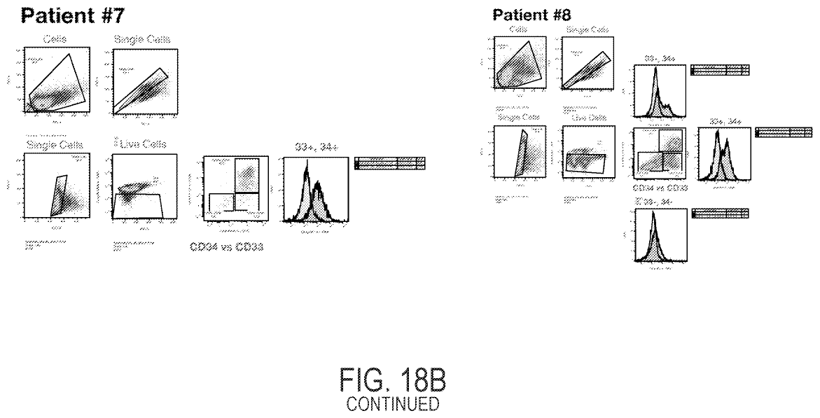

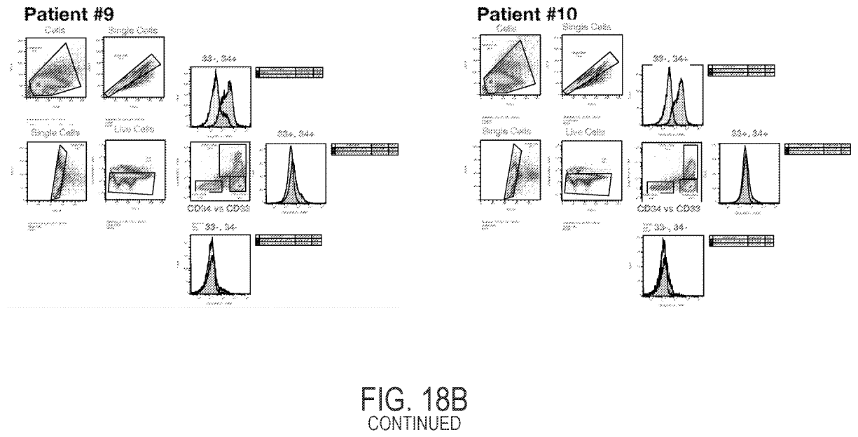

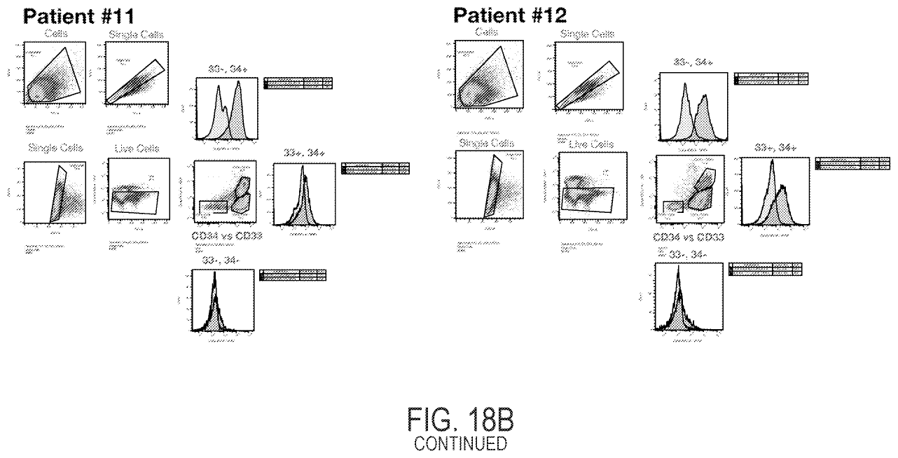

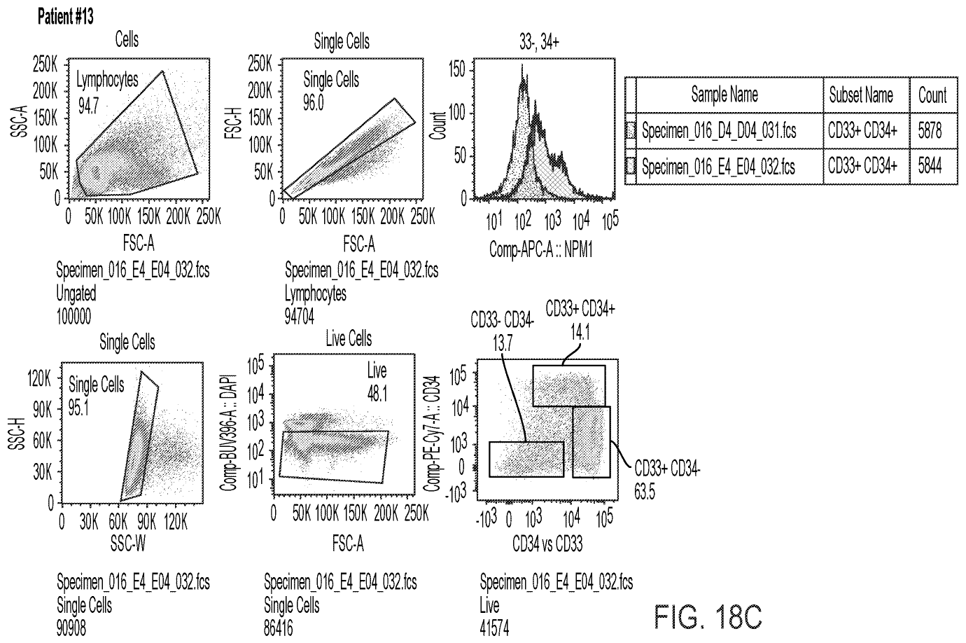

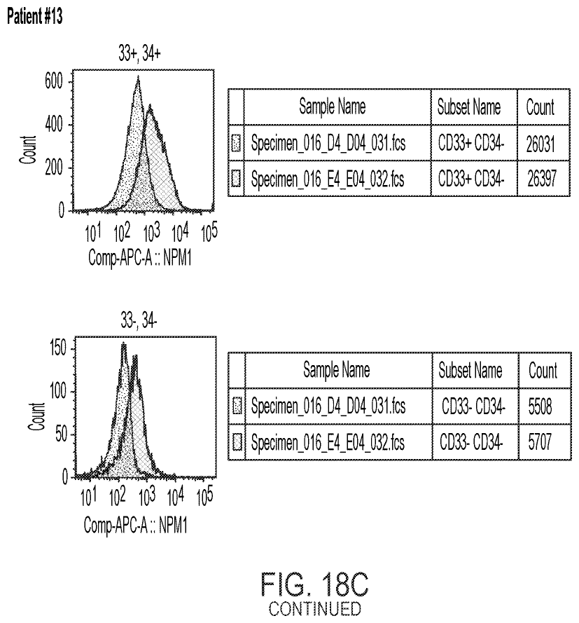

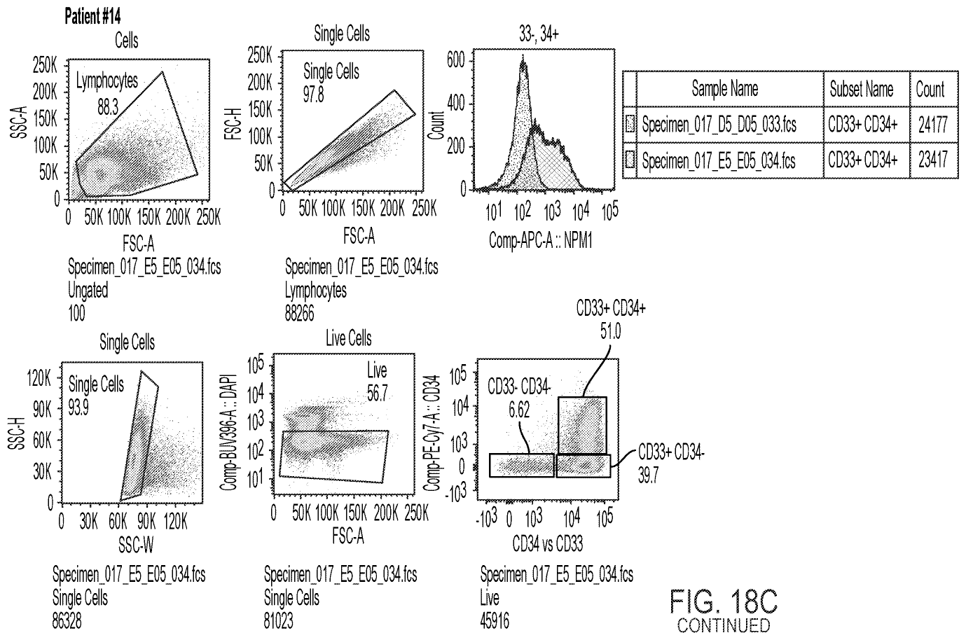

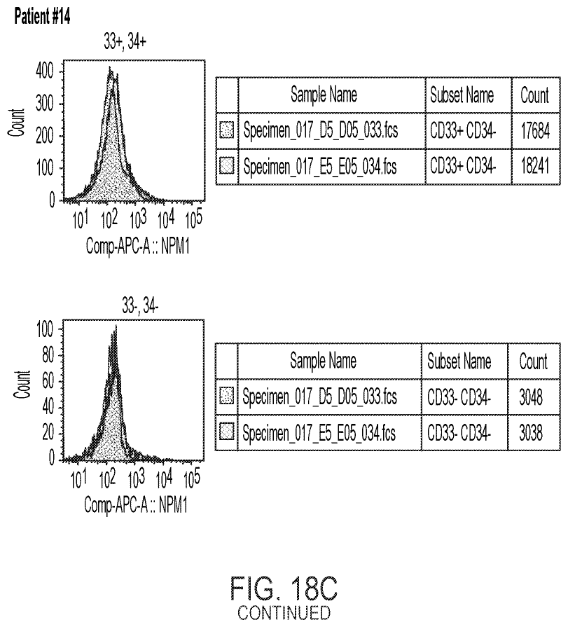

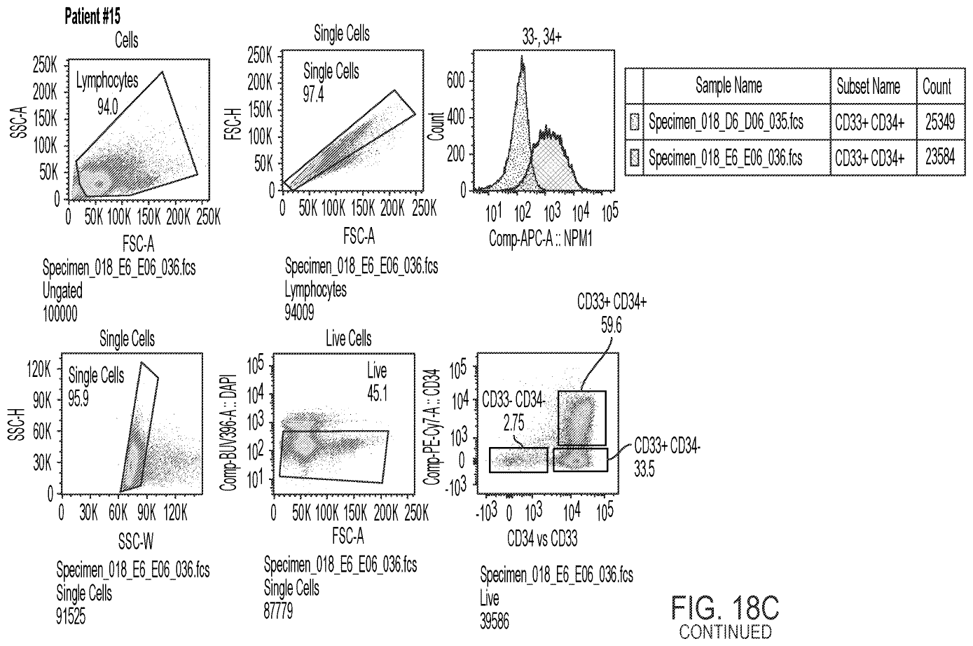

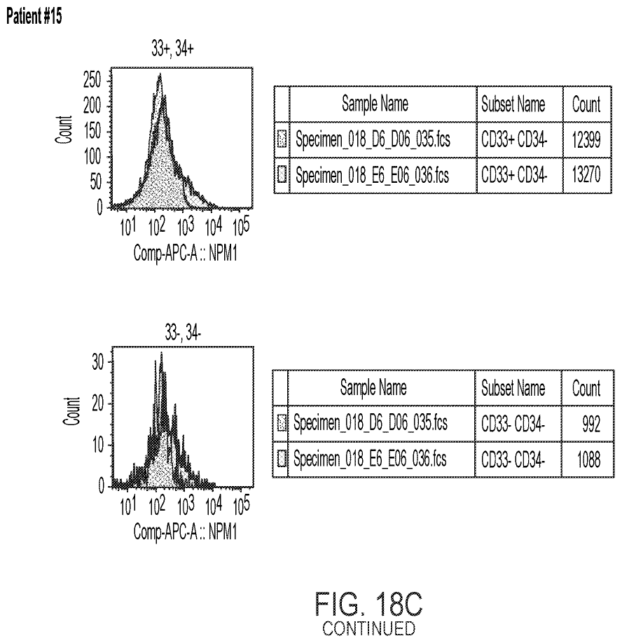

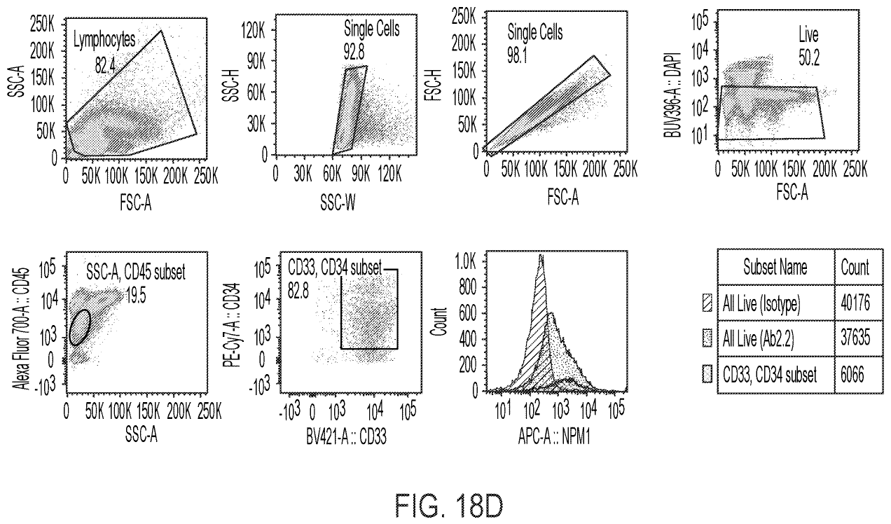

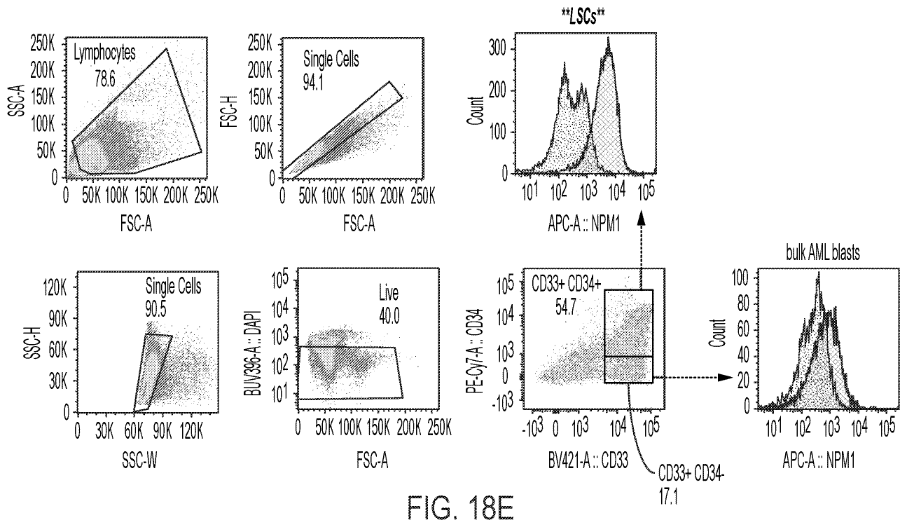

To determine whether Ab2.2 targets NPM1 in cancer cells in vitro patient samples were used. Cells were isolated from the bone marrow of 12 acute myeloid leukemia (AML) patients (FIG. 15) and 15 AML patients (FIG. 17) and analyzed using flow cytometry (FIGS. 16A-16B and FIGS. 18A-18C). The results show that Ab2.2 strongly binds to AML bone marrow blasts (FIGS. 16A-16B, Patient 5) and NPM1c AML bone marrow (FIGS. 18D-18E). Furthermore, NPM1c blasts are CD34-low, allowing examination of LSC population, which are highly bound by Ab2.2. Therefore, Ab2.2 binds best to LSCs in NPM1c patient marrow (FIGS. 18D-18E). The results from the 12 AML patients in FIG. 15 indicate that Ab2.2 strongly stains the blasts, agnostic to mutational status, disease state, or prior treatment. The results from the 15 AML patients in FIG. 17 show that Ab2.2 strongly stains the blasts. Also, in NPM1c patients who's LSCs are CD34−, Ab2.2 strongly stains and therefore Ab2.2 will target leukemia initiating cells.

### Example 8: Ab2.2 does not Cause Toxicity in Mice

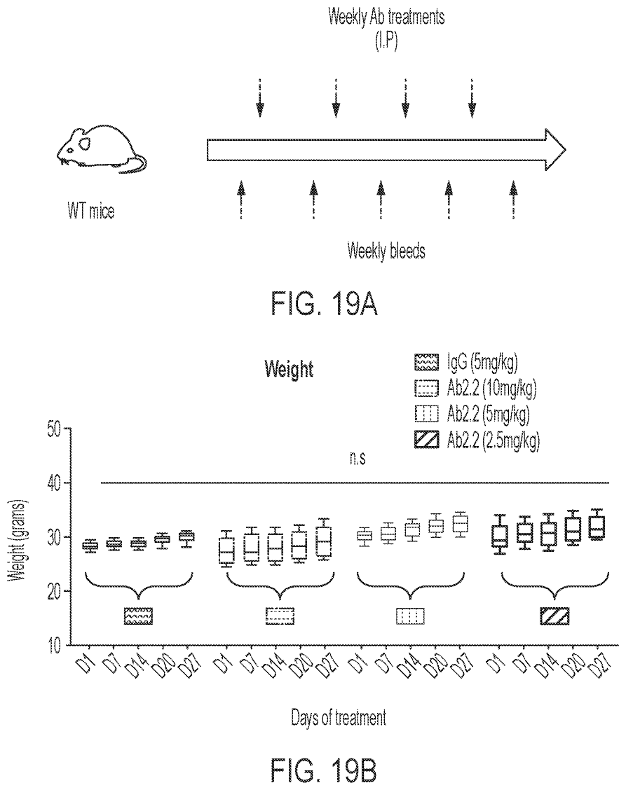

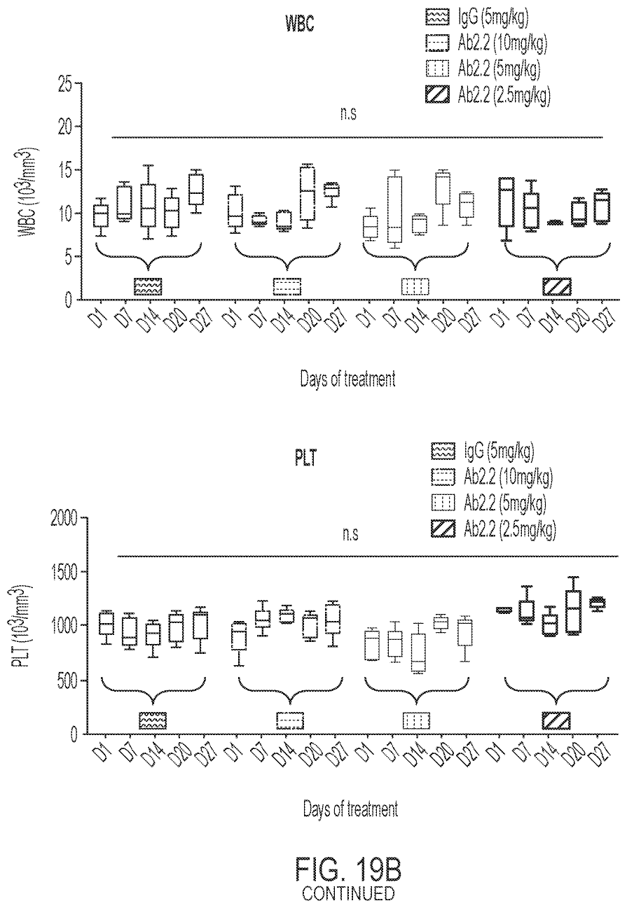

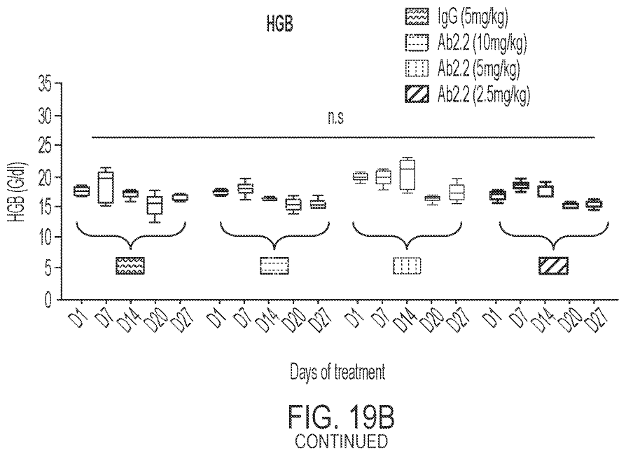

To assess the toxicity of Ab2.2 in vivo, wild-type (WT; C57BL/6J) mice received weekly treatment of Ab2.2. There were 5 mice per group. The Target group received Ab2.2 administered by IP injection at a dose of 2.5 mg/kg, 5 mg/kg, or 10 mg/kg. The control group was administered 5 mg/kg of IgG (FIG. 19A). Four doses total were delivered, one dose per week. Weekly bleeds were performed for sample collection on days (D) 1, 7, 14, 20 and 27 of treatment. On D1, D7, D14, D20 and D27 mice were weighed, and samples were tested for white blood cells (WBC), platelets (PLT) and hemoglobin (HGB). Ab2.2 treatment did not result in observable toxicity in mice (FIG. 19B).

### Example 9: Ab2.2 Treatment Improves Survival in Mouse Model of AML and does not Affect Healthy Mice

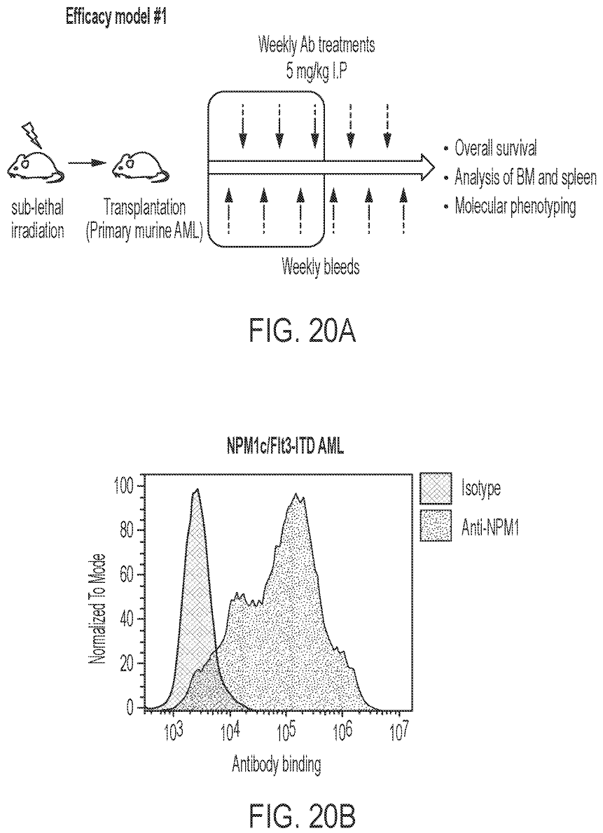

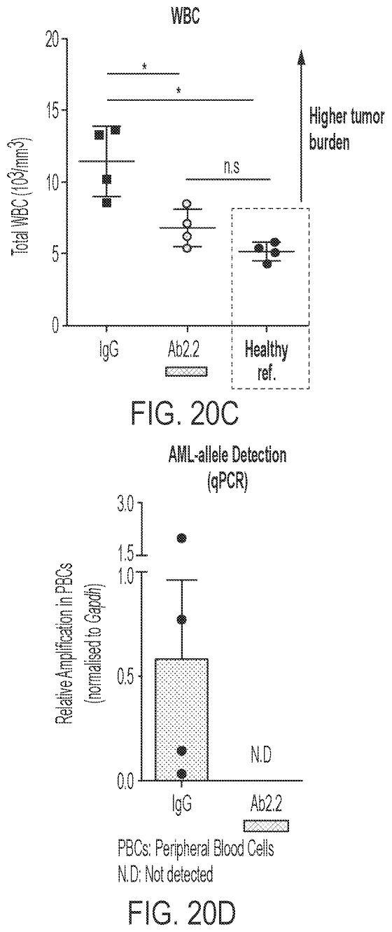

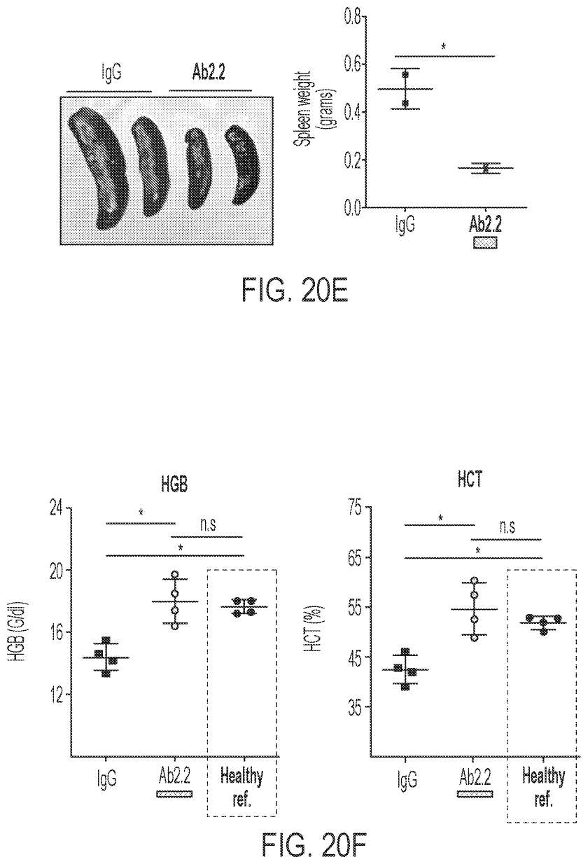

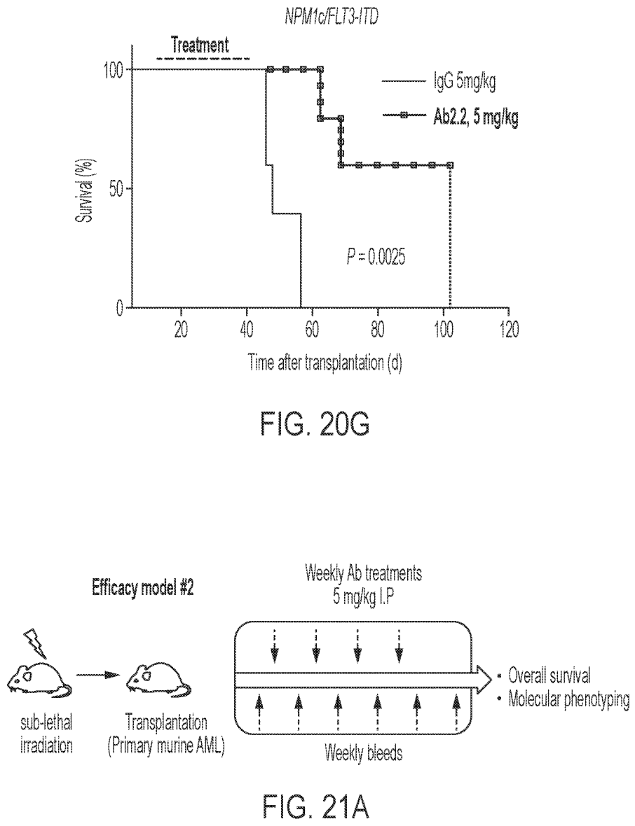

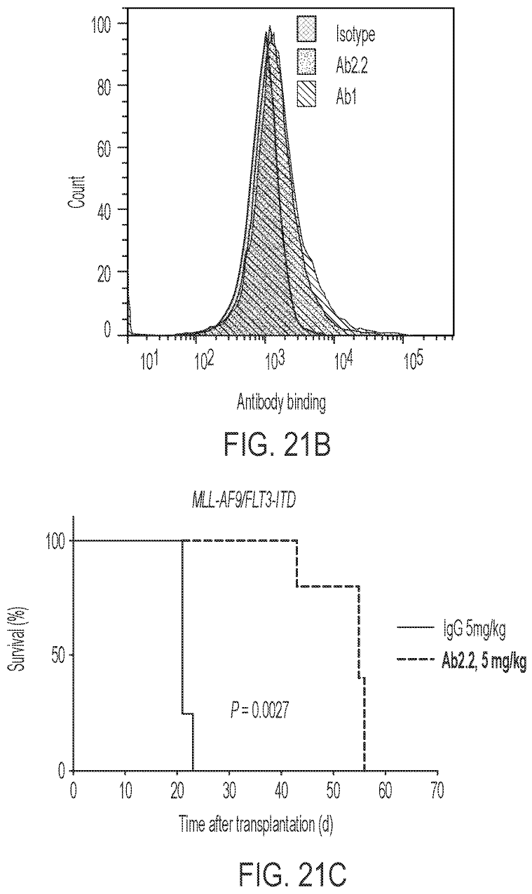

Efficacy of treatment with Ab2.2 was assessed using different models. In efficacy model 1, mice were subjected sub-lethal irradiation, followed by transplantation of primary murine AML cells. The mice receiving the primary murine AML were administered weekly antibody treatments of Ab2.2 at a dose of 5 mg/kg. Administration occurred by IP injection. Weekly bleeds were performed and the overall survival of mice, analysis of bone marrow (BM) and spleen and molecular phenotyping were performed (FIG. 20A). The primary murine cells used were NPM1c/Flt3-ITD AML and a Syngeneic AML mouse model was used. The control group received 5 mg/kg dose (IgG) and the target group received 5 mg/kg dose (Ab2.2). Three doses (1 per week) were administered. Antibody binding was observed and results from the total WBC count and qPCR demonstrate a reduced tumor burden in Ab2.2 treated mice (FIGS. 20B-20D). Also, spleen weight, HGB and hematocrit (HCT) levels demonstrated Ab2.2 resolves defects in hematopoiesis due to AML (FIGS. 21E-21F). Finally, Ab2.2 treatment improved the overall survival of mice (FIG. 20G). The survival study ended at day 100 due to protocol, not toxicity or AML recurrence.

In efficacy model 2, mice were subjected sub-lethal irradiation, followed by transplantation of primary murine AML cells. The mice receiving the primary murine AML was administered weekly antibody treatments of Ab2.2 at a dose of 5 mg/kg. Administration occurred by IP injection. Weekly bleeds were performed and the overall survival of mice, and molecular phenotyping were performed (FIG. 21A). The primary murine cells used for transplantation were MLL-AF9/Flt3-ITD AML. A syngeneic AML mouse model was used. The control group received 5 mg/kg dose (IgG) and the target group received 5 mg/kg dose (Ab2.2). A total of 4 doses of Ab2.2 were administered. Antibody binding was observed (FIG. 21B). The mean survival of the control group was about 20 days, compared to about 55 days in the target group (FIG. 21C). Therefore, Ab2.2 improves overall survival in AML model with lower surface levels of NPM1. Further, these results indicate that any amount of surface expression of NPM1 will lead to survival benefit with Ab2.2 treatment.

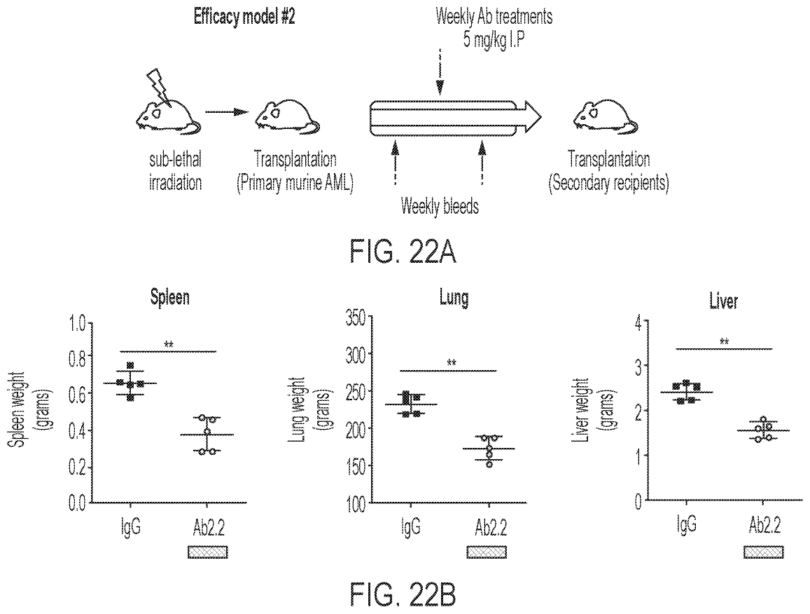

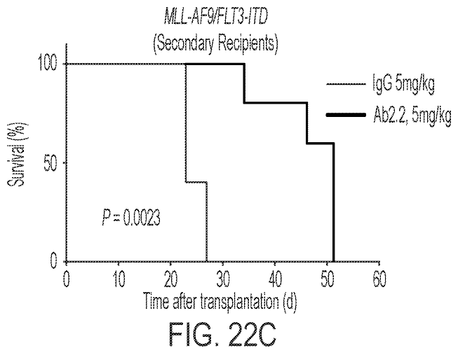

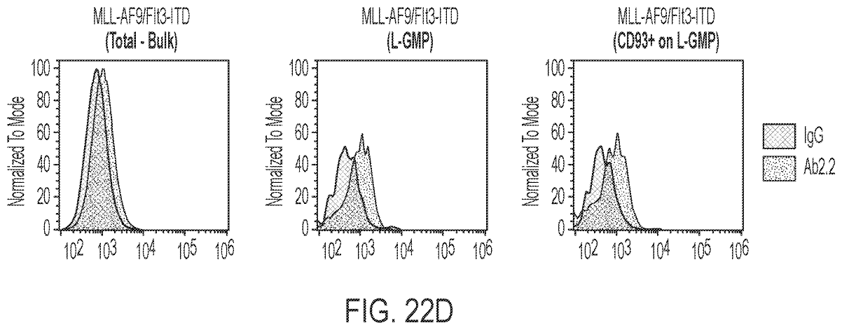

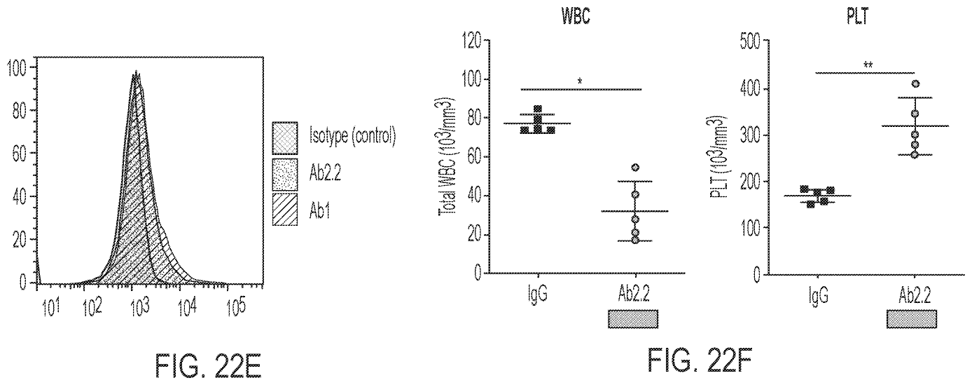

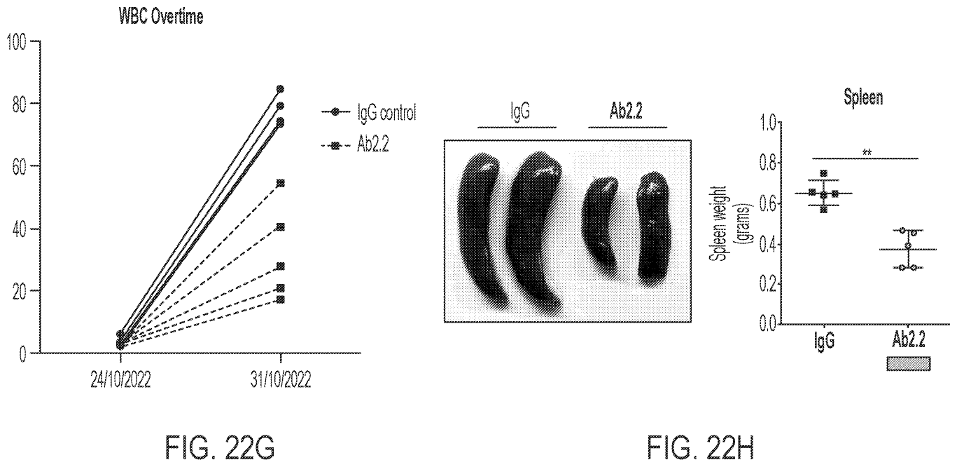

Spleen, lung and liver samples collected three days after the first dose of Ab2.2 treatment in efficacy model 2 were compared between control (IgG) and Ab2.2 treated group. The spleens, lungs and livers of Ab2.2 treated mice all weighed less than the control mice (FIG. 22B). These results indicate that a single dose of Ab2.2 results in robust reduction of organ weight, indicating tumor clearance.

Efficacy model 2 was also used to assess the LSC targeting of Ab2.2 in secondary recipients (FIG. 22A). The transplanted primary murine cells were MLL-AF9/Flt3-ITD AML and a syngeneic AML mouse model was used. The control group received 5 mg/kg dose (IgG) and the target group received 5 mg/kg dose (Ab2.2). One dose of Ab2.2 was administered before secondary recipients received transplantation. Secondary recipients in the control group received transplanted cells from IgG treated mouse. The target group received transplanted cells from Ab2.2 treated mouse. The same number of cells were transplanted, and a survival assay, flow cytometry, examination of engraftment and LSC functional measurement were performed. The results of the survival assay demonstrate that the mean survival of the control group was less than 30 days, compared to about 50 days in the target group (FIG. 22C). The extension of life in the Ab2.2 treated mice demonstrate that fewer stem cells were present in Ab2.2 treated mice. Flow cytometry analysis showed that Ab2.2 stains the stem cell compartment fractionally better than the bulk tumor (FIG. 22D) and MLL-AF9 model has low expression of NPM1 on the surface (FIG. 22E). WBC count was decreased in Ab2.2 treated mice and PLT was increased (FIG. 22F). Further analysis of the WBC over time demonstrated that WBC remain lower in Ab2.2 treated mice compared to IgG treated over time (FIG. 22G). The spleens of Ab2.2 treated secondary recipients weighed less than the IgG treated mice (FIG. 22H). These results indicate that Ab2.2 treatment results in robust reduction of organ weight, indicating tumor clearance. The bone marrow (BM) and peripheral blood (PB) were assessed for percentage (%) of AML. In both the BM and PB there was a reduction in AML % in Ab2.2 treated groups compared to IgG controls (FIG. 22I). These results indicate that Ab2.2 targets tumors after only one dose.

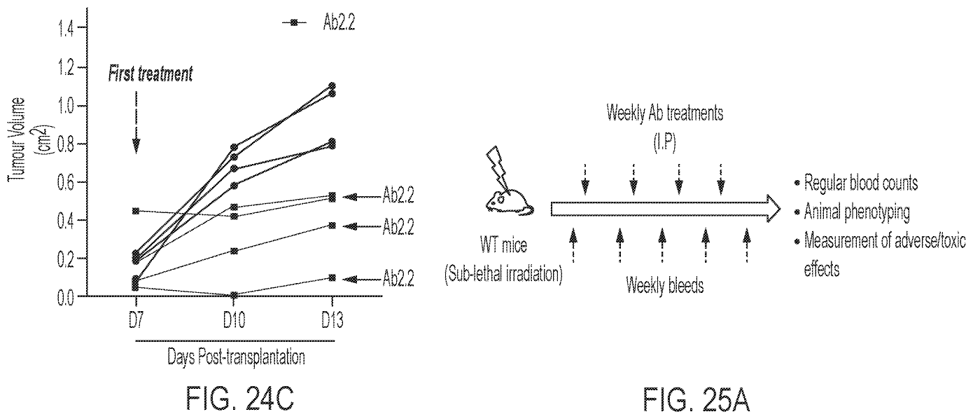

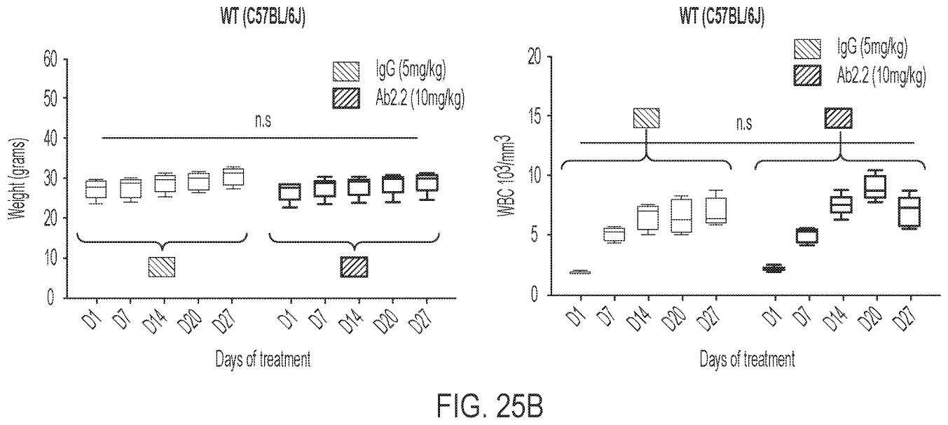

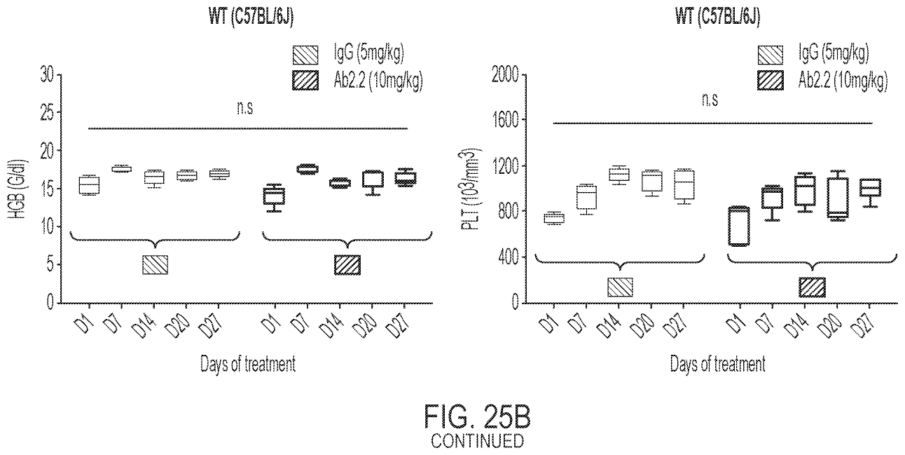

Wild type mice receiving sub-lethal irradiation were tested for effects of Ab2.2 treatment. WT C57BL/6J mice were subjected to sub-lethal irradiation and four treatments of Ab2.2. Ab2.2 treatments occurred weekly via IP injection and weekly bleeds were performed. The control group received 5 mg/kg of IgG and Ab2.2 treated groups received 10 mg/kg of Ab2.2. Regular blood counts, animal phenotyping and measurement of adverse/toxic effects were performed (FIG. 25A). The results show that between day (D) 1 and D27 there was no significant difference observed between the weight, WBC count, HGB levels or PLT levels of IgG treated and Ab2.2 treated mice (FIG. 25B). Therefore, WT mice do not exhibit an observable effect of Ab2.2 treatment.

### Example 10: Ab2.2 Effect on Survival Depends on Immune System

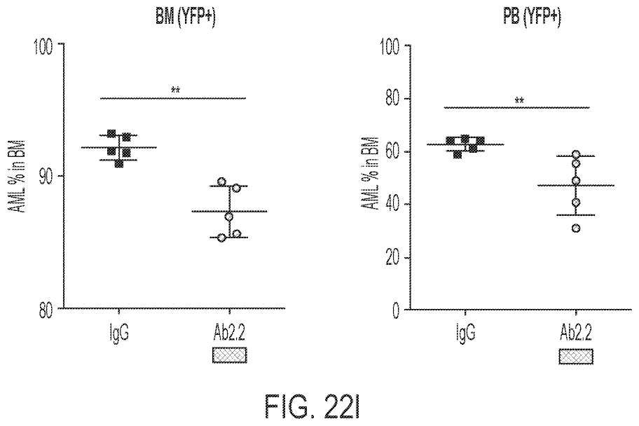

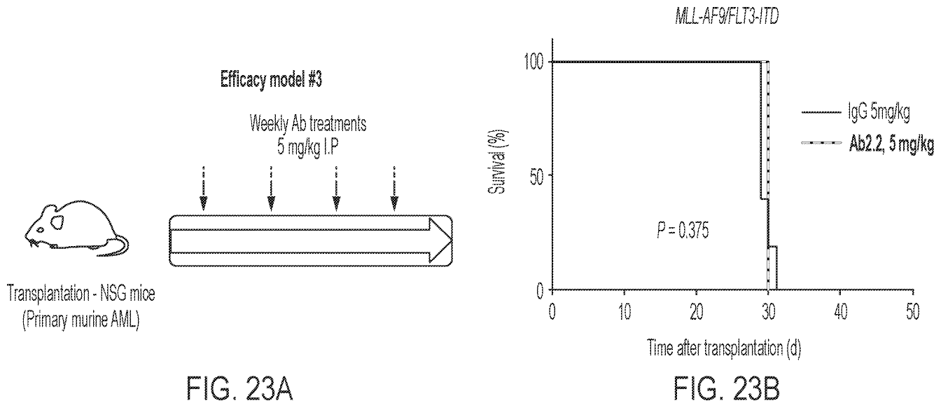

The immune mediated killing ability of Ab2.2 was assessed using efficacy model 3. In efficacy model 3, NSG mice receiving transplantation of primary murine AML cells were given weekly Ab2.2 treatments and assessed for overall survival (FIG. 23A). The transplanted primary murine cells were MLL-AF9/Flt3-ITD AML. The mouse model used was an immunocompromised AML mouse model (NSG). The control group received 5 mg/kg dose (IgG) and the target group received 5 mg/kg dose (Ab2.2). A total of four doses of Ab2.2 were administered. The results of the survival assay demonstrate that the mean survival of the control group and the Ab2.2 treated group were about 30 days (FIG. 23B). Therefore, Ab2.2 does not provide survival benefit without an intact immune system.

### Example 11: Ab2.2 Treatment Reduces Tumor Volume In Vivo

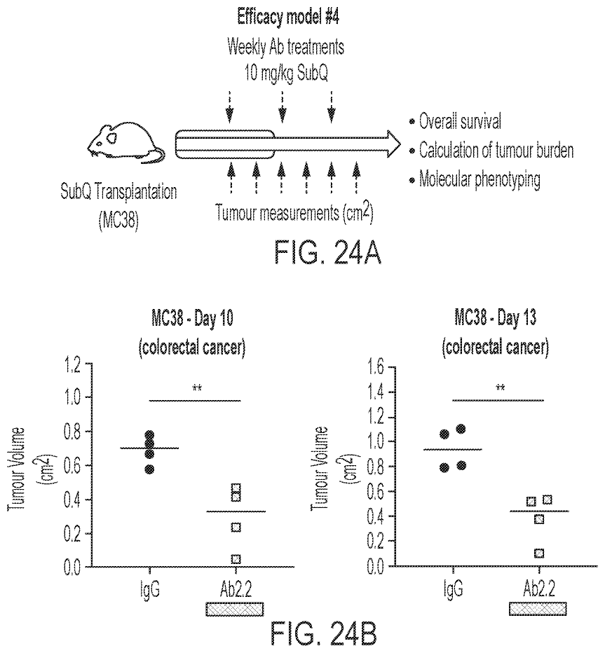

The solid tumor activity of Ab2.2 was assessed in vivo using efficacy model 4. In efficacy model 4, mice that had received transplantation were subjected to weekly treatment of Ab2.2 and assessed for overall survival, calculation of tumor burden and molecular phenotyping (FIG. 24A). The transplanted cells were from a MC38 mouse colorectal adenocarcinoma. A syngeneic mouse model was used. The control group received 10 mg/kg dose (IgG) and the target group received 10 mg/kg dose (Ab2.2). A total of three doses (1 per week) were administered. The results of the calculation of tumor burden demonstrate that on day 10 and day 13 Ab2.2 reduces tumor volume (FIGS. 24B-24C).

### Example 12: Ab2.2 Present on Pre-Cancerous Cells

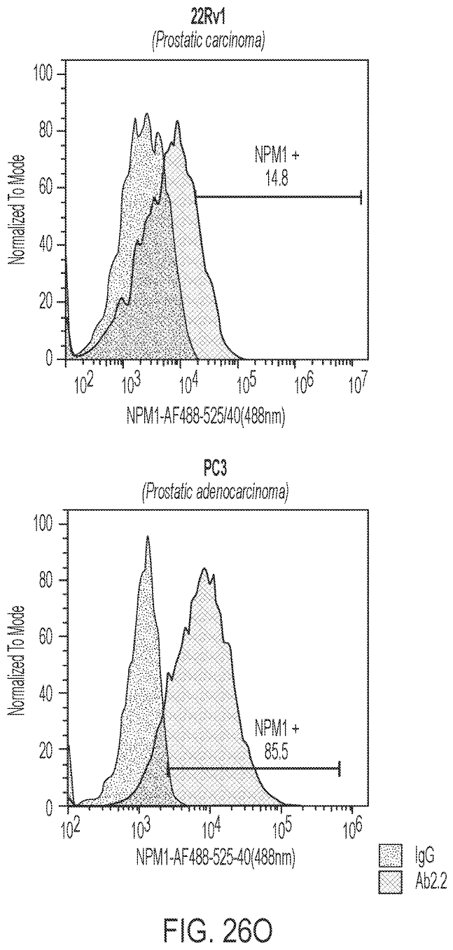

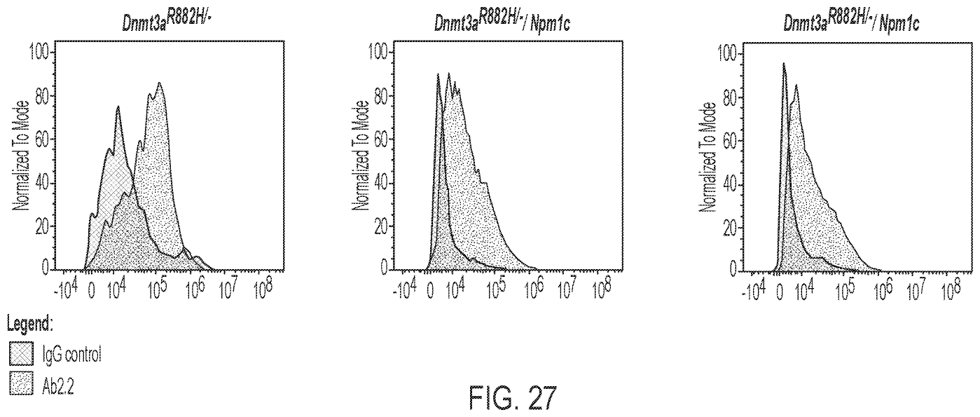

DNMT3a mutations are directly associated with clonal hematopoiesis of indeterminate potential (CHIP) and pre-leukemia. To test whether Ab2.2 could detect Npm1c on Dmt3a R882H mutant cells Dmnt3a^{R882H/-}, and Dmnt3a^{R882H/-}/Npm1c cells were stained with either IgG control or Ab2.2. The results show that there is high surface detection of Npm1c even on single Dmnt3a R882H mutant cells (FIG. 27).

### Example 13: Ab2.2 Binds to Human Tumors In Vitro

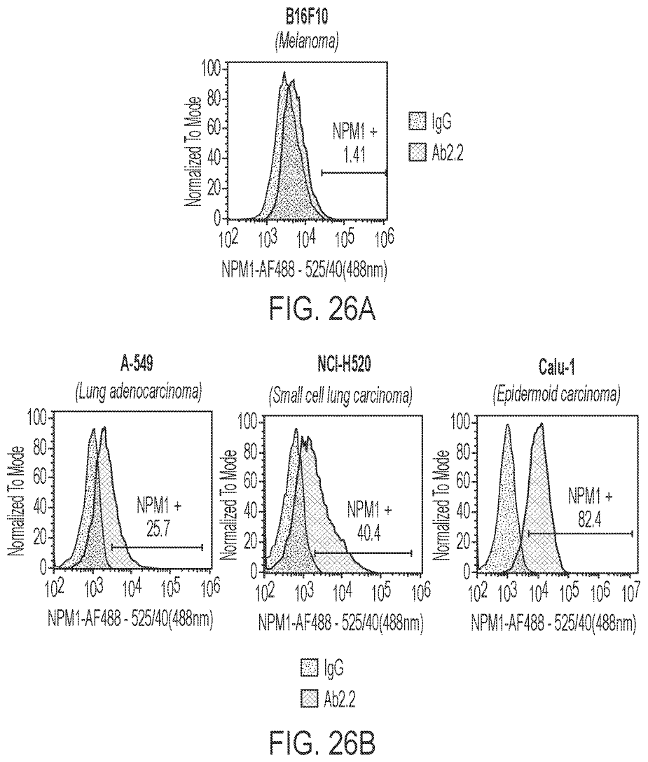

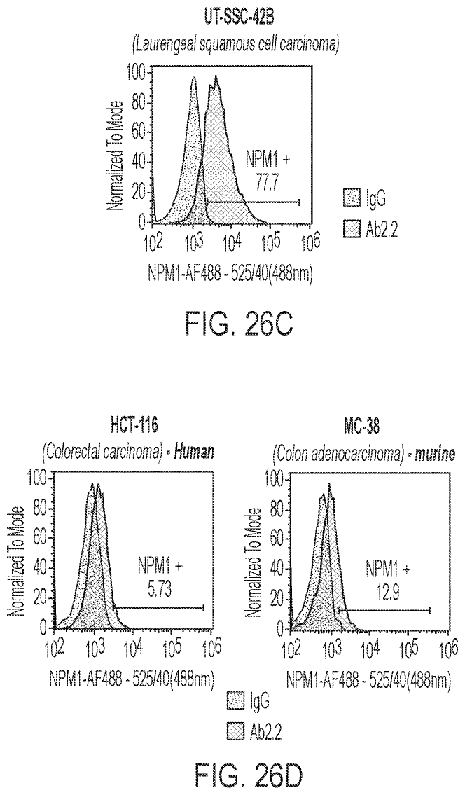

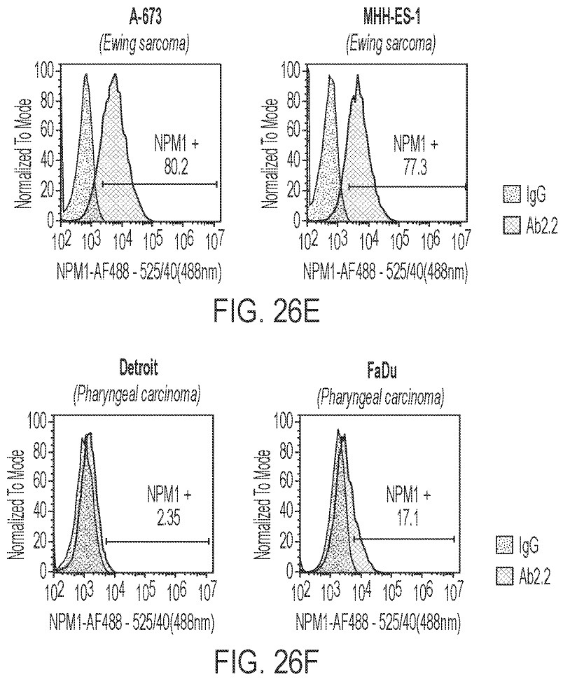

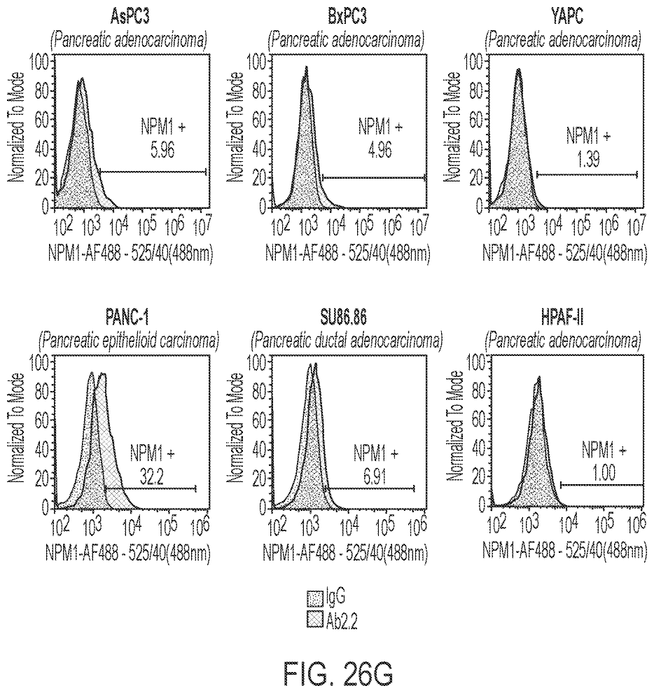

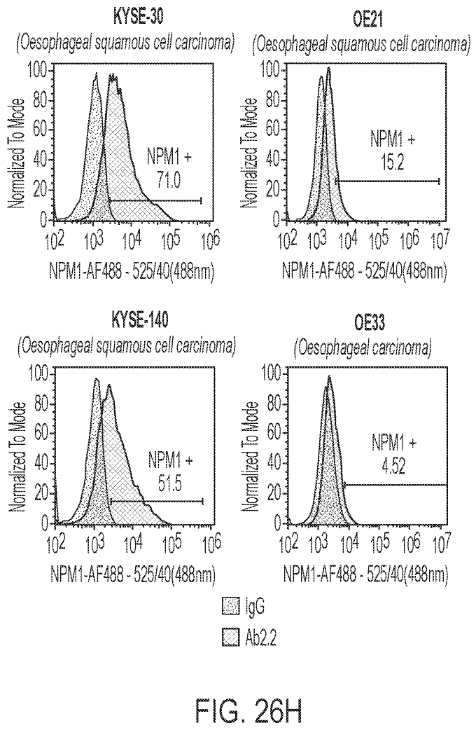

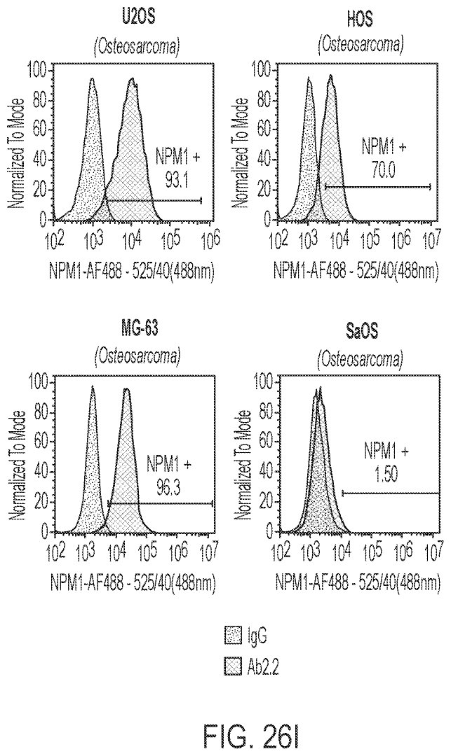

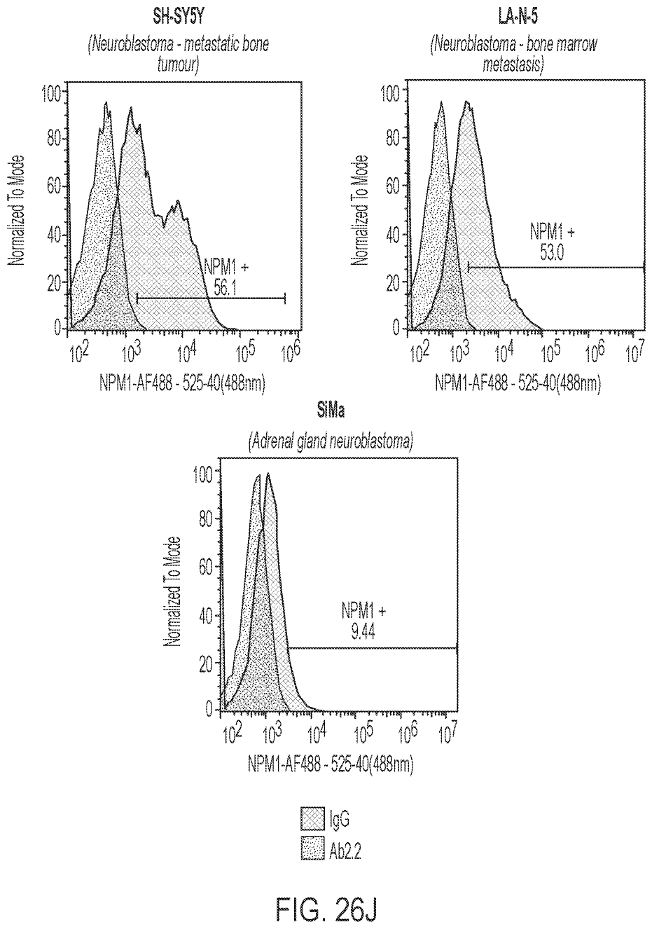

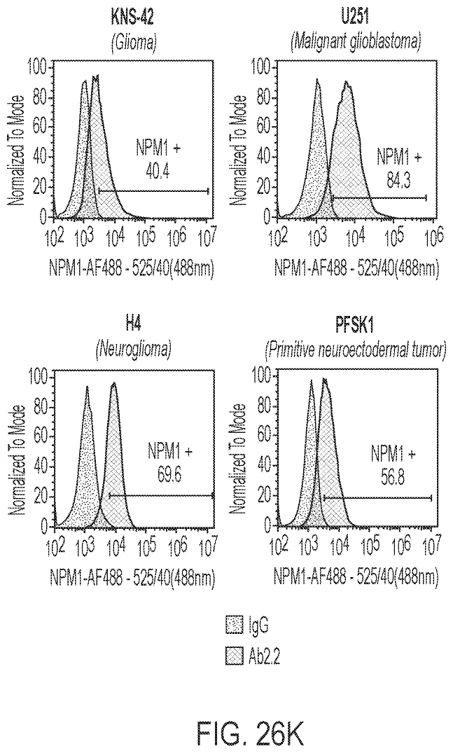

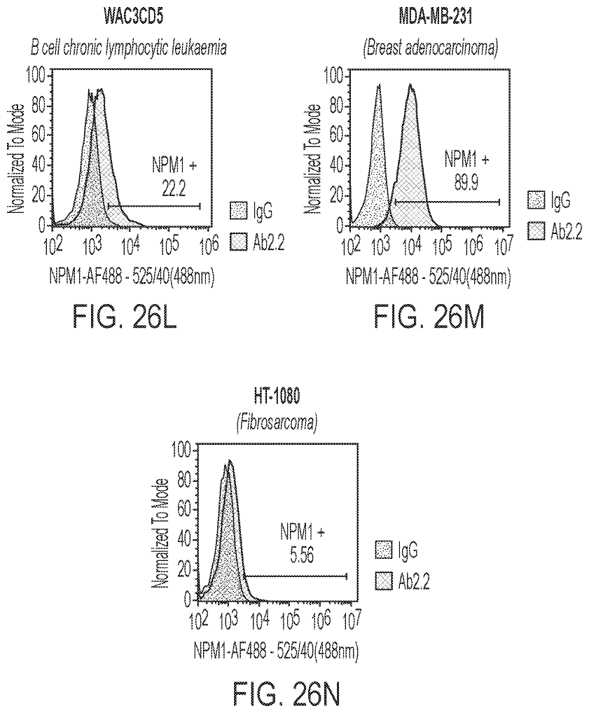

To assess the ability of Ab2.2 to bind to human tumors, in vitro models of various types were investigated using flow cytometry. The tumor models assessed for Ab2.2 binding were murine melanoma, lung carcinoma (human), Laryngeal carcinoma, colorectal (human and murine), Ewing sarcoma (Human), Pharyngeal carcinoma (Human), Pancreatic carcinoma (Human), Oesophageal cancer (Human), Osteosarcoma (Human), Neuroblastomas (Human), Brain tumors (Human), Hematological malignancies, Fibrosarcoma, Prostate cancers, and Pancreatic adenocarcinoma (Murine) (FIGS. 26A-26O). The results show that Ab2.2 binds to a diverse set of human tumor models in vitro.

### Example 14: Ab2.2-Saporin ADC Exhibits In Vitro Activity

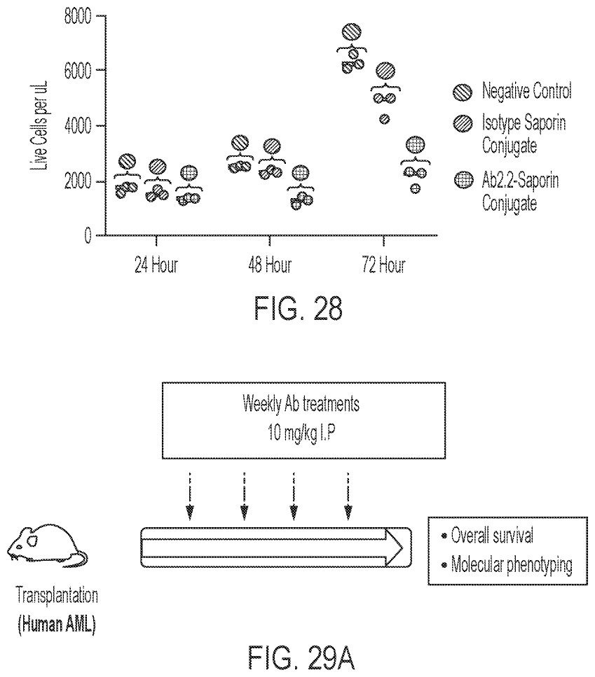

To assess the in vitro activity of Ab2.2 with an ADC OCI-AML3 cells were treated with either a negative control, isotype Saporin conjugate or Ab2.2-Saporin conjugate. Cells were treated over 24, 48 and 72 hours. The number of live cells per μL were assessed at those timepoints. At 72 hours, the number of live cells were about 30% decreased in Ab2.2-Saporin conjugate treated cells compared to negative control (FIG. 28). Therefore, NPM1 can serve as a ADC target in at least some models.

### Example 15: Ab2.2 Treatment Extends Lifespan of Mice Engrafted with Human AML Cell Line

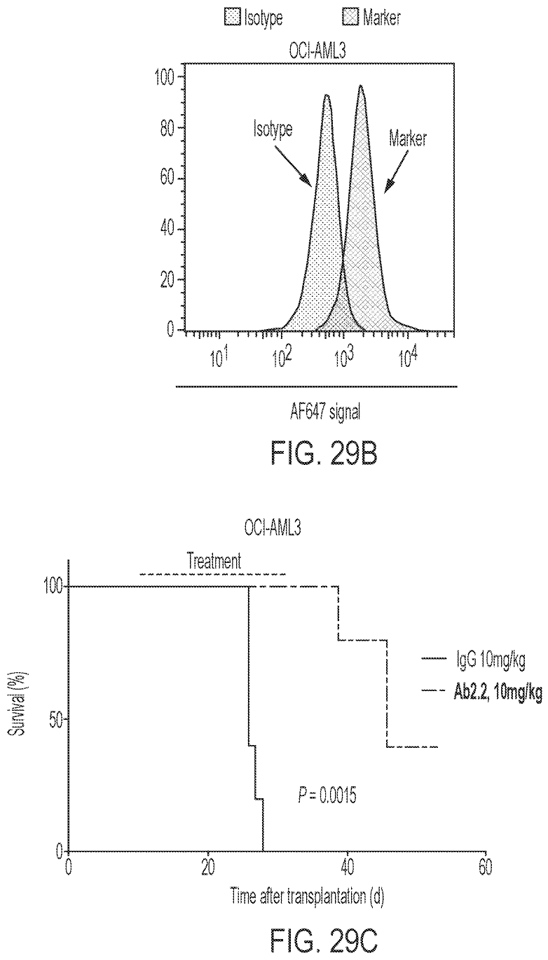

The ability of Ab2.2 to extend lifespan in mouse models xenografted with human AML cell line was assessed. SCID CB17 mice received transplantation of OCI-AML3 cells (human AML). Next, engrafted mice received weekly treatment of Ab2.2 (target group) or IgG (control group). The target group received 10 mg/kg dose of Ab2.2. Mice received a total of 4 doses. Mice were assessed for overall survival, and molecular phenotyping (FIG. 29A).

Antibody binding was assessed using flow cytometry (FIG. 29B). The results of the survival assay demonstrate that survival of engrafted mice treated with Ab2.2 survive for over 40 days after transplantation, significantly longer than IgG treated mice (p=0.0015). (FIG. 29C). Therefore, lifespan extension is observed in mice engrafted with human AML cell line and with natural killer (NK) cells and complement that are treated with Ab2.2.

### Example 16: Ab2.2 Treatment Extends Lifespan of Mice Engrafted with Human AML-PDX Cell Line

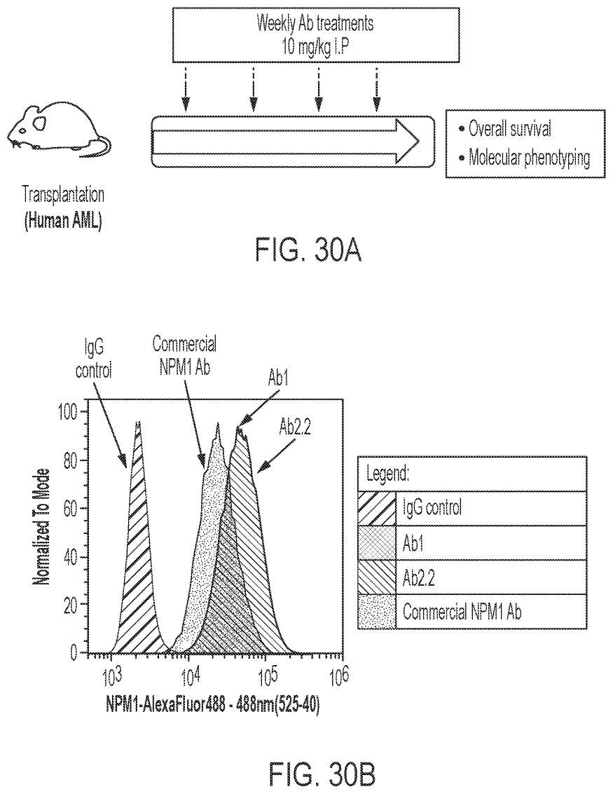

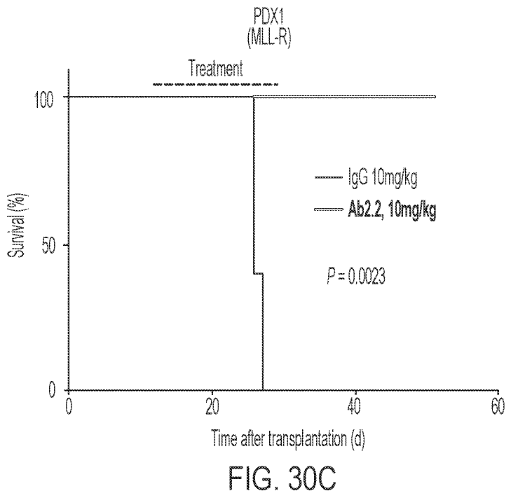

The ability of Ab2.2 to extend lifespan in mouse models xenografted with human AML-PDX was assessed. SCID CB17 mice received transplantation of PDX (MLL-R) cells (human AML-PDX). Next, engrafted mice received weekly treatment of Ab2.2 (target group) or IgG (control group). The target group received 10 mg/kg dose of Ab2.2. Mice received a total of 4 doses. Mice were assessed for overall survival, and molecular phenotyping (FIG. 30A).

Antibody binding was assessed using flow cytometry (FIG. 30B). The results of the survival assay demonstrate that survival of engrafted mice treated with Ab2.2 survive for over 60 days after transplantation, this is significantly longer than IgG treated mice (p=0.0023). (FIG. 30C). Therefore, lifespan extension is observed in mice engrafted with human AML-PDX cell line and with natural killer (NK) cells and complement that are treated with Ab2.2.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. An antibody that binds to nucleophosmin 1 (NPM1).

2. The antibody of claim 1, wherein the antibody comprises:
a heavy chain variable region comprising a heavy chain (HC) complementarity determining region (CDR) 1 comprising the amino acid sequence NIFVH (SEQ ID NO: 1), a HC CDR2 comprising the amino acid sequence KIDPANDNTKFAPNFQG (SEQ ID NO: 2), and a HC CDR3 comprising the amino acid sequence DSSGYDAVDY (SEQ ID NO: 3); and
a light chain variable region comprising a light chain (LC) CDR1 comprising the amino acid sequence RASESVYTYLA (SEQ ID NO: 9), a LC CDR2 comprising the amino acid sequence NAKTLTE (SEQ ID NO: 10), and a LC CDR3 comprising the amino acid sequence QHHYGTPYT (SEQ ID NO: 11).

3. The antibody of claim 2, wherein the heavy chain variable region comprises the amino acid sequence set forth in SEQ ID NO: 28 and/or the light chain variable region comprises the amino acid sequence set forth in SEQ ID NO: 29.

4. The antibody of claim 1, wherein the antibody comprises:
a heavy chain variable region comprising a heavy chain (HC) complementarity determining region (CDR) 1 comprising the amino acid sequence SYAMS (SEQ ID NO: 15), a HC CDR2 comprising the amino acid sequence AISGSGGSTYYADSVKG (SEQ ID NO: 16), and a HC CDR3 comprising the amino acid sequence WRNNAFDY (SEQ ID NO: 17); and
a light chain variable region comprising a light chain (LC) CDR1 comprising the amino acid sequence QGDSLRSYYAS (SEQ ID NO: 22), a LC CDR2 comprising the amino acid sequence GKNNRPS (SEQ ID NO: 23), and a LC CDR3 comprising the amino acid sequence NSSPRLKHRVV (SEQ ID NO: 24).

5. The antibody of claim 4, wherein the heavy chain variable region comprises the amino acid sequence set forth in SEQ ID NO: 30, and/or in the light chain variable region comprises the amino acid sequence set forth in SEQ ID NO: 31.

6. The antibody of any one of claims 1-5, wherein the antibody is a full-length antibody or an antigen-binding fragment thereof.

7. The antibody of claim 6, wherein the antibody is a full-length antibody selected from an immunoglobulin G (IgG), an immunoglobulin A (IgA), an immunoglobulin D (IgD), an immunoglobulin E (IgE), and an immunoglobulin M (IgM).

8. The antibody of claim 7, wherein the antibody is an IgG.

9. The antibody of claim 6, wherein the antibody is an antigen-binding fragment selected from a Fab fragment, a F(ab′)2 fragment, an Ig monomer, a Fd fragment, a scFv, a scAb, a dAb, a Fv, an affibody, a diabody, a single domain heavy chain antibody, and a single domain light chain antibody.

10. The antibody of any one of claims 1-9, wherein the antibody is a human antibody or a humanized antibody.

11. The antibody of any one of claims 1-5, wherein the antibody further comprises a heavy chain constant region.

12. The antibody of claim 11, wherein the heavy chain constant region comprises the amino acid sequence set for in SEQ ID NO: 32 or SEQ ID NO: 46.

13. The antibody of any one of claims 1-5, wherein the antibody further comprises a light chain constant region.

14. The antibody of claim 13, wherein the light chain constant region comprises the amino acid sequence set forth in SEQ ID NO: 33, SEQ ID NO: 34 or SEQ ID NO:47.

15. The antibody of any one of claims 11-14, wherein the antibody comprises a heavy chain comprising the amino acid sequence of SEQ ID NO: 35 and/or a light chain comprising the amino acid sequence of SEQ ID NO: 37.

16. The antibody of any one of claims 11-14, wherein the antibody comprises a heavy chain comprising the amino acid sequence of SEQ ID NO: 36 and/or a light chain comprising the amino acid sequence of SEQ ID NO: 38.

17. The antibody of any one of claims 11-14, wherein the antibody comprises a heavy chain comprising the amino acid sequence of SEQ ID NO: 44 and/or a light chain comprising the amino acid sequence of SEQ ID NO: 45.

18. The antibody of any one of claims 2, 3, and 6-15, wherein the antibody preferentially binds to wild-type NPM1.

19. The antibody of any one of claims 4-14 and 16-17, wherein the antibody preferentially binds to mutant NPM1.

20. A composition comprising the antibody of any one of claims 1-19.

21. The composition of claim 20, wherein the composition further comprises a pharmacologically acceptable excipient.

22. A nucleic acid or nucleic acid set that encodes the antibody of any one of claims 1-19.

23. The nucleic acid or nucleic acid set of claim 22, wherein the nucleic acid or nucleic acid set comprises a vector or vector set.

24. The nucleic acid or nucleic acid set of claim 23, wherein the vector or vector set is an expression vector or vector set.

25. A cell comprising the nucleic acid or nucleic acid set of any one of claims 22-24.

26. A method of producing an antibody, the method comprising:
(i) culturing the cell of claim 25 in culture media under conditions sufficient for expression of the antibody;
(ii) collecting the cultured cells and/or culture media; and
(iii) isolating the antibody from the cultured cells and/or culture media.

27. A conjugate comprising the antibody of any one of claims 1-19 conjugated to an agent.

28. The conjugate of claim 27, wherein the agent is a drug.

29. The conjugate of claim 28, wherein the drug is selected from the group consisting of: auristatin E, auristatin F, monomethyl auristatin D (MMAD), monomethyl auristatin F (MMAF), monomethyl auristatin E (MMAE), actinomycin, actinomycin X2, α-amanitin, β-amanitin, γ-amanitin, ε-amanitin, aeroplysinin, aldoxorubicin, agrochelin, ansatrienin, ansamitocin P-3, aphidicolin, apoptolidin, L-asparaginase, azacitidine, bafilomycin A1, bafilomycin B1, bafilomycin B2, bafilomycin C1, bafilomycin C2, bafilomycin D, bafilomycin E, calicheamicin, campathecin, chaetocin, chaetoglobosin, chlamydocin, cinerubin B, cladribine, colchicine, combretastatin A1, combretastatin A4, cordycepin, cryptophycin, cucurbitacin B, cucurbitacin E, curvulin, cyclopamine, cyclophosphamide, cytarabine, dactinomycin, daunorubicin, decitabine, dexamethasone, dolastatin 10, dolastatin 15, duocarmycin SA, duocarmycin TM, duocarmycin MA, duocarmycin DM, doxorubicin, englerin A, epothilone A, epothilone B, epothilone C, etoposide, fludarabine, fumagillin, geldanamycin, tanespimycin (17-AAG), glucopiericidin A, gramicidin A, herboxidiene, 9-hydroxyellipticine, hydroxyurea, hygrolidin, hypothemycin, idarubicin, ilimaquinone, isatropolone A, isofistularin-3, ixabepilone, JW55, lactacystin, luisol A, maytansinol, mertansine (DM1), maytansine DM3, ravtansine (DM4), maytansinoid AP-3, mechercharmycin A, mensacarcin, methotrexate, 6-mercaptopurine, microcolin B, microcystin LR, mitoxantrone, muscotoxin A, myoseverin, mytoxin B, nelarabine, nemorubicin, nocuolin A, okilactomycin, oligomycin A, oligomycin B, paclitaxel, larotaxel, milataxel, ortataxel, tesetaxel, phallacidin, phalloidin, phytosphingosine, piericidin A, pironetin, podophyllotoxin, polyketomycin, prednisone, pseudolaric acid B, pseurotin A, puwainaphycin F, pyrrolobenzodiazepine, quinaldopeptin, rachelmycin, rebeccamycin, Ro 5-3335, safracin B, sandramycin, sanguinarine, saporin, sinefungin, taltobulin, telomestatin, 6-thioguanine, thiocolchicine, tolytoxin, tripolin A, triptolide, tubastatin A, tubulysin A, tubulysin M, tubulysin IM-1, tubulysin IM-2, tubulysin IM-3, venetoclax, and vincristine.

30. The conjugate of claim 29, wherein the drug is saporin, daunorubicin, venetoclax, or azacitidine.

31. The conjugate of any one of claims 27-30, wherein the antibody and the drug are conjugated via a linker.

32. The conjugate of claim 31, wherein the linker is a cleavable linker.

33. The conjugate of claim 32, wherein the linker is a pH-sensitive linker, a glutathione-sensitive linker, or a protease-cleavable linker.

34. The conjugate of claim 32 or 33, wherein the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, CL2A, maleimidocaproyl (MC), and Mal-EBE-Mal.

35. The conjugate of claim 31, wherein the linker is a non-cleavable linker.

36. The conjugate of claim 35, wherein the non-cleavable linker is selected from the group consisting of: N-succinimidyl 4-(N-maleimidomethyl)cyclohexane-1-carboxylate (SMCC) and maleimidomethyl cyclohexane-1-carboxylate (MCC), MC-VC-PAB.

37. The conjugate of any one of claims 28-36, wherein the ratio of the antibody to the drug is between 1:1 and 1:10.

38. The conjugate of claim 37, wherein the ratio of the antibody to the agent is 1:4.

39. The conjugate of claim 27, wherein the agent is a radioisotope.

40. The conjugate of claim 39, wherein the radioisotope is selected from the group consisting of: Iodine-131, Rhenium-188, Yttrium-90, Bismuth-213, and Actinium-225.

41. The conjugate of claim 27, wherein the agent is an imaging agent.

42. The conjugate of claim 41, wherein the imaging agent is a luminescent or fluorescent imaging agent.

43. The conjugate of claim 41, wherein the imaging agent is an agent that is detectable by magnetic resonance imaging (MRI).

44. The conjugate of claim 43, wherein the imaging agent is gadolinium-diethylenetriamine (Gd-DTPA).

45. A method of treating a nucleophosmin 1 (NPM1)-expressing cancer, the method comprising administering to a subject in need thereof an effective amount of the antibody of any one of claims 1-19 or the conjugate of any one of claims 27-40.

46. The method of claim 45, wherein the NPM1-expressing cancer is a cancer in which NPM1 is expressed on the surface of cancer cells.

47. The method of claim 46, wherein the NPM1-expressing cancer is a cancer in which mutant NPM1 is expressed on the surface of cancer cells.

48. The method of any one of claims 45-47, wherein the cancer is a solid or liquid cancer selected from the group consisting of: a hematological cancer, a lung cancer, a breast cancer, a brain cancer, a gastrointestinal cancer, a liver cancer, a kidney cancer, a bladder cancer, a pancreatic cancer, an ovarian cancer, a testicular cancer, a prostate cancer, an endometrial cancer, a muscle cancer, a bone cancer, a neuroendocrine cancer, a connective tissue cancer, a head or neck cancer, or a skin cancer.

49. The method of any one of claims 45-48, wherein the cancer is selected from the group consisting of: acute myeloid leukemia (AML), acute promyeloid leukemia (APL), acute lymphoblastic leukemia (ALL), non-Hodgkin's lymphoma, and myelodysplastic syndrome (MDS).

50. The method of any one of claims 45-49, wherein the cancer is a metastatic cancer.

51. The method of any one of claims 45-50, wherein the cancer is a therapy-related cancer or a secondary malignancy.

52. The method of claim 51, wherein the cancer is therapy-related AML (t-AML) or a secondary malignancy of non-Hodgkin's lymphoma.

53. The method of any one of claims 45-52, wherein the antibody or conjugate is administered systemically or locally.

54. The method of claim 53, wherein the antibody or conjugate is administered orally or via injection.

55. The method of claim 54, wherein the injection is intravenous injection, subcutaneous injection, intraperitoneal injection, or intratumoral injection.

56. The method of any one of claims 45-55, wherein the administration occurs more than once.

57. The method of claim 56, wherein the administration occurs between once per day and once per six months.

58. The method of any one of claims 45-57, wherein the subject is a mammal.

59. The method of claim 58, wherein the subject is a human.

60. A method for treating a disease associated with a cell expressing cell surface nucleophosmin 1 (NPM1), the method comprising administering to a subject in need thereof an effective amount of an antibody or a conjugate that binds to NPM1.

61. The method of claim 59, wherein the antibody is the antibody of any one of claims 1-19 and the conjugate is the conjugate of any one of claims 27-40.

62. A method of evaluating the presence of a nucleophosmin 1 (NPM1)-expressing cancer, the method comprising:
(i) administering to a subject in need thereof an effective amount of the conjugate of any one of claims 41-44;
(ii) imaging the conjugate in the subject; and
(iii) determining the presence of a NPM1-expressing cancer in the subject, based on the level and position of the antibody conjugate imaged in the subject.

63. The method of claim 62, wherein the NPM1-expressing cancer is a cancer in which NPM1 is expressed on the surface of cancer cells.

64. The method of claim 63, wherein the NPM1-expressing cancer is a cancer in which mutant NPM1 is expressed on the surface of cancer cells.

65. The method of any one of claims 62-64, wherein the cancer is selected from the group consisting of: acute myeloid leukemia (AML), acute promyeloid leukemia (APL), acute lymphoblastic leukemia (ALL), non-Hodgkin's lymphoma, and myelodysplastic syndrome (MDS).

66. The method of any one of claims 62-65, wherein the cancer is a metastatic cancer.

67. The method of any one of claims 62-66, wherein the cancer is a therapy-related cancer or a secondary malignancy.

68. The method of claim 67, wherein the cancer is therapy-related AML (t-AML) or a secondary malignancy of non-Hodgkin's lymphoma.

69. The method of any one of claims 62-68, wherein the imaging is luminescent or fluorescent imaging.

70. The method of any one of claims 62-69, wherein the imaging is magnetic resonance imaging (MRI).

71. The method of any one of claims 62-70, further comprising administering to the subject an effective amount of the antibody of any one of claims 1-19 or the conjugate of any one of claims 27-40, if a NPM1-expressing cancer is determined to be present in the subject.

72. A method of evaluating the presence of a nucleophosmin 1 (NPM1)-expressing cancer, the method comprising:
(i) collecting a biological sample from a subject in need thereof;
(ii) contacting the biological sample with the conjugate of any one of claims 41-44;
(iii) analyzing binding between the conjugate and NPM1-expressing cancer cells in the biological sample; and
(iv) determining the presence of a NPM1-expressing cancer in the subject, based on the level of binding between the conjugate and NPM1-expressing cancer cells analyzed in the sample.

73. The method of claim 72, wherein the NPM1-expressing cancer is a cancer in which NPM1 is expressed on the surface of cancer cells.

74. The method of claim 73, wherein the NPM1-expressing cancer is a cancer in which mutant NPM1 is expressed on the surface of cancer cells.

75. The method of any one of claims 72-74, wherein the cancer is selected from the group consisting of: acute myeloid leukemia (AML), acute promyeloid leukemia (APL), acute lymphoblastic leukemia (ALL), non-Hodgkin's lymphoma, and myelodysplastic syndrome (MDS).

76. The method of any one of claims 72-75, wherein the cancer is a metastatic cancer.

77. The method of any one of claims 72-76, wherein the cancer is a therapy-related cancer or a secondary malignancy.

78. The method of claim 77, wherein the cancer is therapy-related AML (t-AML) or a secondary malignancy of non-Hodgkin's lymphoma.

79. The method of any one of claims 72-78, wherein the biological sample is a blood sample, a serum sample, or a plasma sample.

80. The method of any one of claims 72-79, wherein the analysis is luminescent or fluorescent analysis.

81. The method of any one of claims 72-80, wherein the analysis is performed via flow cytometry.

82. The method of any one of claims 72-81, further comprising administering to the subject an effective amount of the antibody of any one of claims 1-19 or the conjugate of any one of claims 27-40, if a NPM1-expressing cancer is determined to be present in the subject.


SEQUENCE LISTING (48 sequences)

SEQ ID NO: 1 - protein, 5 aa, Homo sapiens
NIFVH

SEQ ID NO: 2 - protein, 17 aa, Homo sapiens
KIDPANDNTKFAPNFQG

SEQ ID NO: 3 - protein, 10 aa, Homo sapiens
DSSGYDAVDY

SEQ ID NO: 4 - protein, 7 aa, Homo sapiens
GFNIKNI

SEQ ID NO: 5 - protein, 6 aa, Homo sapiens
DPANDN

SEQ ID NO: 6 - protein, 6 aa, Homo sapiens
KNIFVH

SEQ ID NO: 7 - protein, 13 aa, Homo sapiens
WIGKIDPANDNTK

SEQ ID NO: 8 - protein, 11 aa, Homo sapiens
ASDSSGYDAVD

SEQ ID NO: 9 - protein, 11 aa, Homo sapiens
RASESVYTYLA

SEQ ID NO: 10 - protein, 7 aa, Homo sapiens
NAKTLTE

SEQ ID NO: 11 - protein, 9 aa, Homo sapiens
QHHYGTPYT

SEQ ID NO: 12 - protein, 7 aa, Homo sapiens
YTYLAWY

SEQ ID NO: 13 - protein, 10 aa, Homo sapiens
LLVYNAKTLT

SEQ ID NO: 14 - protein, 8 aa, Homo sapiens
QHHYGTPY

SEQ ID NO: 15 - protein, 5 aa, synthetic construct
SYAMS

SEQ ID NO: 16 - protein, 17 aa, synthetic construct
AISGSGGSTYYADSVKG

SEQ ID NO: 17 - protein, 8 aa, synthetic construct
WRNNAFDY

SEQ ID NO: 18 - protein, 7 aa, synthetic construct
GFTFSSY

SEQ ID NO: 19 - protein, 6 aa, synthetic construct
SGSGGS

SEQ ID NO: 20 - protein, 6 aa, synthetic construct
SSYAMS

SEQ ID NO: 21 - protein, 9 aa, synthetic construct
AKWRNNAFD

SEQ ID NO: 22 - protein, 11 aa, synthetic construct
QGDSLRSYYAS

SEQ ID NO: 23 - protein, 7 aa, synthetic construct
GKNNRPS

SEQ ID NO: 24 - protein, 11 aa, synthetic construct
NSSPRLKHRVV

SEQ ID NO: 25 - protein, 7 aa, synthetic construct
SYYASWY

SEQ ID NO: 26 - protein, 10 aa, synthetic construct
LVIYGKNNRP

SEQ ID NO: 27 - protein, 10 aa, synthetic construct
NSSPRLKHRV

SEQ ID NO: 28 - protein, 119 aa, synthetic construct
EVQLQQSVAELVRPGASVKLSCTASGFNIKNIFVHWVKQRPEQGLEWIGKIDPANDNTKFAPNFQGKAAISADTSSNTAYLQLSSLTSEDTAIYYCASDSSGYDAVDYWGQGTSVTVSS

SEQ ID NO: 29 - protein, 107 aa, synthetic construct
DIQMTQSPASLSASVGETVTITCRASESVYTYLAWYQQKQGKSPQLLVYNAKTLTEGVPSRFSGSGSGTQFSLRINSLQPEDFGSYYCQHHYGTPYTFGSGTKLEIK

SEQ ID NO: 30 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWRNNAFDYWGQGTLVTVSS

SEQ ID NO: 31 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSSPRLKHRVVFGGGTKLTVL

SEQ ID NO: 32 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 34 - protein, 106 aa, synthetic construct
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 35 - protein, 469 aa, synthetic construct
MYRMQLLSCIALSLALVTNSEVQLQQSVAELVRPGASVKLSCTASGFNIKNIFVHWVKQRPEQGLEWIGKIDPANDNTKFAPNFQGKAAISADTSSNTAYLQLSSLTSEDTAIYYCASDSSGYDAVDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 36 - protein, 467 aa, synthetic construct
MYRMQLLSCIALSLALVTNSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWRNNAFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 37 - protein, 234 aa, synthetic construct
MYRMQLLSCIALSLALVTNSDIQMTQSPASLSASVGETVTITCRASESVYTYLAWYQQKQGKSPQLLVYNAKTLTEGVPSRFSGSGSGTQFSLRINSLQPEDFGSYYCQHHYGTPYTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - protein, 234 aa, synthetic construct
MYRMQLLSCIALSLALVTNSSSELTQDPAVSVALGQTVRITCQGDSLRSYYASWYQQKPGQAPVLVIYGKNNRPSGIPDRFSGSSSGNTASLTITGAQAEDEADYYCNSSPRLKHRVVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 39 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 40 - protein, 292 aa, Mus musculus
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEDEEDVKLLGMSGKRSAPGGGNKVPQKKVKLDEDDEDDDEDDEDDEDDDDDDFDEEETEEKVPVKKSVRDTPAKNAQKSNQNGKDLKPSTPRSKGQESFKKQEKTPKTPKGPSSVEDIKAKMQASIEKGGSLPKVEAKFINYVKNCFRMTDQEAIQDLWQWRKSL

SEQ ID NO: 41 - protein, 294 aa, unidentified
  source 1..294: note = Primate
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEDDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 42 - protein, 43 aa, Homo sapiens
QASIEKGGSLPKVEAKFINYVKNCFRMTDQEAIQDLWQQRKSL

SEQ ID NO: 43 - protein, 45 aa, synthetic construct
QASIEKGGSLPKVEAKFINYVKNCFRMTDQEAIQDLAVEEVSLRK

SEQ ID NO: 44 - protein, 470 aa, synthetic construct
MDPKGSLSWRILLFLSLAFELSYGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWRNNAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 234 aa, synthetic construct
MSVPTQVLGLLLLWLTDARCSSELTQDPAVSVALGQTVRITCQGDSLRSYYASWYQQKPGQAPVLVIYGKNNRPSGIPDRFSGSSSGNTASLTITGAQAEDEADYYCNSSPRLKHRVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - protein, 13 aa, synthetic construct
WVSAISGSGGSTY